(19) United States
(12) Patent Application Publication
(10) Pub. No.: US 2011/0130403 A1
(43) Pub. Date: Jun. 2, 2011

(54) PYRAZOLO [3, 4-B] PYRIDINE DERIVATIVES AS PHOSPHODIESTERASE INHIBITORS

(75) Inventors: Sonali Rudra, Haryana (IN); Nidhi Gupta, Delhi (IN); Lalit Kumar Baregama, Rajasthan (IN); Ritu Agarwal, West Bengal (IN); Mandadapu Raghuramaiah, Andhra Pradesh (IN); Vinayak Vasantrao Khairnar, Maharashtra (IN); Venkata P. Palle, Maharashtra (IN); Sarala Balachandran, Maharashitra (IN); Abhijit Ray, Delhi (IN); Sunada G. Dastidar, Delhi (IN); Lalitha Vijaykrishnan, Delhi (IN)

(73) Assignee: RANBAXY LABORATORIES LIMITED, Gurgaon, Haryana (IN)

(21) Appl. No.: 12/531,045

(22) PCT Filed: Mar. 13, 2008
(86) PCT No.: PCT/IB2008/050941

§ 371 Date: Jan. 20, 2010

(30) Foreign Application Priority Data
Mar. 14, 2007 (IN) 549/DEL/2007

(51) Int. Cl. A61K 31/437; C07D 471/04; A61K 31/444; A61K 31/497; A61K 31/506; A61K 31/53; A61P 29/00; A61P 37/06; A61P 25/00

(52) U.S. Cl. 514/242; 546/119; 514/303; 544/405; 514/255.05; 544/331; 514/275; 544/182

(57) ABSTRACT

The present invention relates to phosphodiesterase (PDE) type 4, phosphodiesterase (PDE) type 7 and dual PDE type 4/PDE type 7 inhibitors. Compounds disclosed hereinf having the structure of Formula I: can be useful in the treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans. Processes for the preparation of disclosed compounds, pharmaceutical compositions containing the disclosed compounds and their use as PDE type 4, PDE type 7 and dual PDE t e 4/PDE t e 7 inhibitors are rovided.

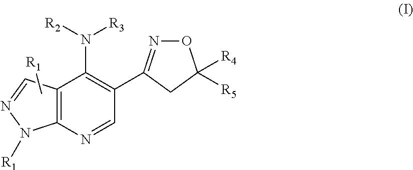

### FIELD OF THE INVENTION

The present invention relates to phosphodiesterase (PDE) type 4, phosphodiesterase (PDE) type 7 and dual PDE type 4/PDE type 7 inhibitors.

Compounds disclosed herein can be useful in the treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Processes for the preparation of disclosed compounds, pharmaceutical compositions containing the disclosed compounds and their use as PDE type 4, PDE type 7 and dual PDE type 4/PDE type 7 inhibitors are provided.

### BACKGROUND OF THE INVENTION

It is known that cyclic adenosine-3′,5′-monophosphate (cAMP) exhibits an important role of acting as an intracellular secondary messenger (Pharmacol. Rev., 12, (1960), 265). Its intracellular hydrolysis to adenosine 5′-monophosphate (AMP) causes number of inflammatory conditions which are not limited to COPD, asthma, arthritis, psoriasis, allergic rhinitis, shock, atopic dermatitis, Crohn's disease, adult respiratory distress syndrome (ARDS), eosinophilic granuloma, allergic conjunctivitis, osteoarthritis or colitis. PDE4 inhibitors are designed to inhibit the activity of PDE4, the enzyme which breaks down neuronal cAMP. Studies have shown that administering PDE4 inhibitors can have a restorative effect on memory loss in animal models, including those of Alzheimer's disease (Expert Opin. Ther. Targ., 9 (6): (2005) 1283-1305; Drug Discovery Today, 10, 22: (2005), 1503-1519). The most important role in the control of cAMP (as well as of cGMP (cyclic guanosine monophosphate)) level is played by cyclic nucleotide phosphodiesterases (PDE) which represent a biochemically and functionally highly variable super family of enzymes. Eleven distinct families of cyclic nucleotide phosphodiesterases with more than 25 gene products are currently recognized. Although PDE1, PDE2, PDE3, PDE4, and PDE7 all use cAMP as a substrate, only PDE4 and PDE7 are highly selective for hydrolysis of cAMP. Inhibitors of PDE, particularly the PDE4 inhibitors, such as rolipram or Ro-1724 are therefore known as cAMP-enhancers. Immune cells contain type 4 and type 3 PDE, the PDE4 type being prevalent in human mononuclear cells. Thus the inhibition of phosphodiesterase type 4 has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. Distinct classes of PDE's have been recognized (TIPS, 11, (1990), 150), and their selective inhibition has led to improved drug therapy (TIPS, 12, (1991), 19). Thus it was recognized that inhibition of PDE4 could lead to inhibition of inflammatory mediator release (J. Mol. Cel. Cardiol., 12 (Suppl. II), (1989), S 61) and airway smooth muscle relaxation.

The current approach of targeting PDE4 for alleviating the chronic inflammation associated with COPD is compromised by the dose limiting side effects that are proving difficult to overcome. Theoretically, an alternate strategy would be to use small molecule inhibitors to target other members of the cAMP dependent PDE family that share a common pulmonary cellular distribution to PDE4. It is hypothesized that such an approach would yield compounds with an improved therapeutic ratio. Of the novel cAMP family of proteins discovered so far, PDE7A offers itself as a promising candidate because of its cellular distribution in almost all pro inflammatory and immune cells (Curr Pharm Des., 12, (2006), 1-14). Additionally, it has been shown to be a prime modulator of human T cell function as well (Science; 283 (5403): Feb. 5, (1999), 848-851).

Thus, dual specificity inhibitors that target both PDE4 and PDE7 would in principle, have an improved spectrum and a wider therapeutic window in the clinics. Compounds with dual PDE4 and PDE7 inhibitory effects have been shown to inhibit T cell function such as cytokine production, proliferation and activation of CD25 expression markers on T cells induced by antigen stimulation (Eur. J. Pharmacol., 541, (2006), 106-114). Development of dual PDE4-PDE7 inhibitors would yield a novel class of drugs blocking T cell component of a disease partly through PDE7 inhibition as well as possess anti-inflammatory activity. (Eur. J. Pharmacol., 550, (2006), 166-172 Eur. J. Pharmacol., 559, (2007), 219-226). More importantly, such a pharmacophore would be less limited by nausea and vomiting, a major side effect associated with PDE4 inhibition.

WO 03/047520 discloses substituted aminomethyl compounds and derivatives thereof, which have been described to be useful as inhibitors of factor Xa. WO 00/59902 discloses aryl sulfonyls, which have been described to be useful as inhibitors of factor Xa. WO 97/48697 discloses substituted azabicyclic compounds and their use as inhibitors of the production of TNF and cyclic AMP phosphodiesterase. WO 98/57951 and U.S. Pat. No. 6,339,099 describe nitrogen containing heteroaromatics and derivatives, which have been said to be the inhibitors of factor Xa. WO 2005/063767 and WO 2006/001894 disclose indoles, 1H-indazoles, 1,2-benzisoxazoles, and 1,2-benzisothiazoles, preparation and uses thereof. WO 2007/031838 discloses substituted pyrazolo[3,4-b]pyridines as phosphodiesterase inhibitors.

### SUMMARY OF THE INVENTION

The present invention provides phosphodiesterase (PDE) type 4, PDE type 7 and dual PDE type 4/PDE type 7 inhibitors, which can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Pharmaceutically acceptable salts, pharmaceutically acceptable solvates, stereoisomers, tautomers, racemates, regioisomers, geometric isomers, prodrugs, metabolites, polymorphs or N-oxides of these compounds having the same type of activity are also provided.

Pharmaceutical compositions containing the compounds, which may also contain pharmaceutically acceptable carriers or diluents, can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Other aspects will be set forth in the accompanying description which follows and in part will be apparent from the description or may be learnt by the practice of the invention.

In accordance with one aspect, there are provided compounds having the structure of Formula I:

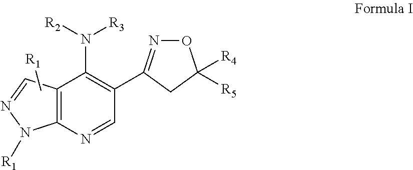

or their pharmaceutically acceptable salts, pharmaceutically acceptable solvates, stereoisomers, tautomers, racemates, regioisomers, geometric isomers, prodrugs, metabolites, polymorphs or N-oxides, wherein R₁ can be hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ and R₃ independently can be hydrogen, aryl, heteroaryl,

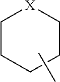

or

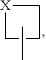

wherein X can be CH₂, CO, O, CH(CH₂)ₙ(OH), CH(COOR_{f}), or S(O)ₙ, (wherein n can be an integer from 0-2 and R_{f} can be hydrogen, alkyl, alkenyl, cycloalkyl, aryl, aralkyl, heterocyclyl, heteroaryl, (heterocyclyl)alkyl or (heteroaryl)alkyl); and
R₄ and R₅ independently can be alkyl, —CN, —(CH₂)ₙC(═O)NR_{f}R_{q} {wherein n can be an integer from 0-2 and R_{f} and R_{q} independently can be hydrogen, alkyl, alkenyl, cycloalkyl, aryl, aralkyl, heterocyclyl, heteroaryl, (heterocyclyl)alkyl, (heteroaryl)alkyl or R_{f} and R_{q} taken together with the nitrogen atom to which they are attached can form a optionally substituted heterocyclyl ring}, —(CH₂)ₙC(═O)OR_{f} {wherein n and R_{f} are the same as defined earlier} or —(CH₂)ₙ₁OR_{f} {wherein n1 can be an integer from 0-3 and R_{f} is the same as defined earlier}.

In another aspect, there are provided methods for treating, preventing, inhibiting or suppressing inflammatory diseases, CNS diseases or autoimmune diseases, in a mammal, comprising administering a therapeutically effective amount of a PDE type 7 inhibitor or dual PDE type 4/PDE type 7 inhibitor having the structure of Formula Ia,

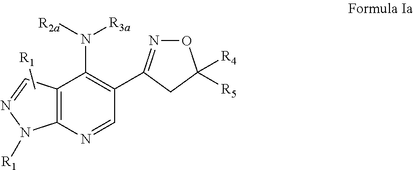

or its pharmaceutically acceptable salts, pharmaceutically acceptable solvates, stereoisomers, tautomers, racemates, regioisomers, geometric isomers, prodrugs, metabolites, polymorphs or N-oxides, wherein R₁ can be hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ₐ can be hydrogen, alkyl, alkenyl, alkynyl, acyl, cycloalkyl, aryl, aralkenyl, aralkyl, (cycloalkyl) alkyl, heterocyclyl, heteroaryl, (heterocyclyl)alkyl or (heteroaryl) alkyl;
R₃ₐ can be cyclopropyl, cyclopentyl, alkyl, alkenyl, alkynyl, acyl, aralkenyl, aralkyl, (cycloalkyl) alkyl, (heterocyclyl)alkyl or (heteroaryl) alkyl;
R₄ and R₅ independently can be alkyl, —CN, —(CH₂)ₙC(═O)NR_{f}R_{q} {wherein n can be an integer from 0-2 and R_{f} and R_{q} independently can be hydrogen, alkyl, alkenyl, cycloalkyl, aryl, aralkyl, heterocyclyl, heteroaryl, (heterocyclyl)alkyl or (heteroaryl)alkyl}, —(CH₂)ₙC(═O)OR_{f} {wherein n and R_{f} are the same as defined earlier}, —(CH₂)ₙ₁R_{f} {wherein n1 can be an integer from 0-3 and R_{f} is the same as defined earlier}.

In another aspect, there are provided methods for the treatment, prevention, inhibition or suppression of multiple sclerosis, AIDS, rejection of transplant, rheumatoid arthritis, bronchitis, chronic obstructive pulmonary disease (COPD), asthma, psoriasis, allergic rhinitis, shock, atopic dermatitis, Crohn's disease, adult respiratory distress syndrome (ARDS), eosinophilic granuloma, allergic conjunctivitis, osteoarthritis, colitis, pancreatitis, and cancer in a mammal comprising administering a therapeutically effective amount of a PDE type 7 inhibitor or dual PDE type 4/PDE type 7 inhibitor having the structure of Formula Ia.

The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR₊—, wherein R_{α} can be hydrogen, alkyl, cycloalkyl, alkenyl, cycloalkenyl, alkynyl, aryl, acyl, aralkyl, —C(═O)OR_{λ}, SOₙR_{ψ} (wherein n is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl) or —C(═O)NR_{λ}R_{π} {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆ alkyl), —CH═N—NH(C₁₋₆alkyl), —CH═N—N(C₁₋₆alkyl)C₁₋₆alkyl, arylthio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, —C(═O)heterocyclyl, —O—C(═O)NR_{λ}R_{π}, nitro or —SOₙR_{ψ} (wherein R_{λ}, R_{π}, n and R_{ψ} are the same as defined earlier). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₙR_{ψ}. Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, hydroxy, alkoxy, halogen, CF₃, cyano, and —SOₙR_{ψ} (wherein R_{λ}, R_{π}, n and R_{ψ} are the same as defined earlier); or an alkyl group as defined above that has substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₙR_{ψ} (wherein R_{λ}, R_{π}, n and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π} and —SOₙR_{ψ} (wherein R_{λ}, R_{π}, n and R_{ψ} are as defined earlier). Groups, such as ethenyl or vinyl (CH═CH₂), 1-propylene or allyl (—CH₂CH═CH₂), iso-propylene (—C(CH₃)═CH₂ and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₙR_{ψ} (wherein R_{λ}, R_{π}, n and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, cyano or —SOₙR_{ψ} (wherein R_{λ}, R_{π}, n and R_{ψ} are the same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentyl, cyclohexyl and the like or multiple ring structures, including adamantanyl, and bicyclo [2.2.1] heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₙR_{ψ} (wherein R_{λ}, R_{π}, n and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —NHC(═O)NR_{λ}R_{π}, —OC(═O)NR_{λ}R_{π}, cyano or —SOₙR_{ψ} (wherein R_{λ}, R_{π}, n and R_{ψ} are the same as defined earlier).

The term “(cycloalkyl) alkyl” refers to alkyl-cycloalkyl group linked through alkyl portion, wherein the alkyl and cycloalkyl are as defined earlier.

The term “alkoxy” denotes the group O-alkyl, wherein alkyl is the same as defined above.

The term “aryl,” unless otherwise specified, refers to a monocyclic aromatic system having 6 to 14 carbon atoms, wherein the ring system can be mono-, bi- or tricyclic and carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₙR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}, R_{π}, n and R_{ψ} are the same as defined earlier). Aryl groups optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl and aryl are as defined above). Examples of aralkyl groups include benzyl, ethylphenyl, propylphenyl, naphthylmethyl, p-methoxybenzyl and the like.

The term “aralkenyl,” unless otherwise specified, refers to alkenyl-aryl linked through alkenyl (wherein alkenyl and aryl are as defined above) portion.

The term “aryloxy” denotes the group O-aryl, wherein aryl is as defined above.

The term “cycloalkoxy” denotes the group O-cycloalkyl, wherein cycloalkyl is as defined above.

The term “carboxy,” as defined herein, refers to —C(═O)OR_{f}, wherein R_{f} is the same as defined above.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S and optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}—NHC(═O)NR_{λ}R_{π}, —SOₙR_{ψ}, —O—C(═O)NR_{λ}R_{π}, —O—C(═O)R_{λ}, or —O—C(═O)OR_{λ} (wherein n, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic cycloalkyl group having 5 to 10 atoms wherein 1 to 4 carbon atoms in a ring are replaced by heteroatoms selected from O, S, SO(O) or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, aralkyl, cyano, nitro, oxo, carboxy, optionally substituted heterocyclyl, optionally substituted heterocyclylalkyl, optionally substituted heteroaryl, —(CH₂)ₙOH (wherein n is an integer from 0-2), —O—C(═O)R_{λ}, —O—C(═O)OR_{λ}, —C(═O)NR_{λ}R_{π}, SOₙR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein n, R_{ψ}, R_{λ} and R_{π} are as defined earlier) or guanidine. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include tetrahydropyranyl, oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, isoindole 1,3-dione, pyrrolidinyl, piperidinyl, piperazinyl, 3,6-diazabicyclo[3.1.0]hex-6-yl, 3-azabicyclo[3.1.0]hex-6-yl, 3H-imidazo[4,5-b]pyridine, isoquinolinyl, 1H-pyrrolo[2,3-b]pyridine or piperazinyl and the like.

“(Heteroaryl) alkyl” refers to alkyl-heteroaryl group linked through alkyl portion, wherein the alkyl and heteroaryl are as defined earlier.

“(Heterocyclyl) alkyl” refers to alkyl-heterocyclyl group linked through alkyl portion, wherein the alkyl and heterocyclyl are as defined earlier.

“Acyl” refers to —C(═O)R_{z} (wherein R_{z} is alkyl, cycloalkyl, aryl, aralkyl, heteroaryl, heterocyclyl, heteroarylalkyl or heterocylylalkyl).

“Amine,” unless otherwise specified, refers to —NH₂. “Substituted amine” unless otherwise specified, refers to a group —N(Rₖ)₂ wherein each Rₖ is independently selected from the group hydrogen provided that both Rₖ groups are not hydrogen (defined as “amino”), alkyl, alkenyl, alkynyl, aralkyl, cycloalkyl, aryl, heteroaryl, heterocyclyl, heterocyclylalkyl, heteroarylalkyl, acyl, S(O)ₘR_{ψ} (wherein m and R_{ψ} are the same as defined above), —C(═Rᵥ)NR_{λ}R_{π} (wherein Rᵥ is O or S and R_{λ} and R_{π} are the same as defined earlier) or NHC(═Rᵥ)NR_{π}R_{λ} (wherein Rᵥ, R_{π} and R_{λ} are the same as defined earlier). Unless otherwise constrained by the definition, all amino substituents may optionally be further substituted by 1-3 substituents chosen from alkyl, aralkyl, cycloalkyl, aryl, heteroaryl, heterocyclyl, carboxy, —COOR_{ψ}, hydroxy, alkoxy, halogen, CF₃, cyano, —C(═Rᵥ)NR_{λ}R_{π}, —O(C═O)NR_{λ}R_{π}, —OC(═Rᵥ)NR_{λ}R_{y} (wherein R_{λ}, R_{π} and Rᵥ are the same as defined earlier), —S(O)ₘR_{ψ} (wherein R_{ψ} and m are the same as defined above).

“Thiocarbonyl” refers to —C(═S)H. Thiocarbonyl may be substituted and “substituted thiocarbonyl” refers to —C(═S)R′″, wherein R′″ is selected from alkyl, cycloalkyl, aryl, aralkyl, heteroaryl, heterocyclyl, heteroarylalkyl, heterocyclylalkyl, amine or substituted amine. Unless otherwise constrained by the definition, all substituents optionally may be substituted further by 1-3 substituents selected from alkyl, aralkyl, cycloalkyl, aryl, heteroaryl, heterocyclyl, carboxy, hydroxy, alkoxy, halogen, CF₃, cyano, —C(═O)NR_{λ}R_{π}, —O—C(═O)NR_{λ}R_{π} and —SOₙR_{ψ} (wherein R_{λ}, R_{π}, n and R_{ψ} are as defined earlier).

The term “oxo” means “═O”. Oxo is attached at a carbon atom unless otherwise noted. Oxo, together with the carbon atom to which it is attached forms a carbonyl group (i.e., C═O).

The term “halogen” refers to fluorine, chlorine, bromine or iodine.

The compounds of the present invention can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

In accordance with yet another aspect, there are provided processes for the preparation of the compounds as described herein.

### DETAILED DESCRIPTION OF THE INVENTION

The compounds described herein may be prepared by techniques well known in the art and familiar to the average synthetic organic chemist. In addition, the compounds of present invention may be prepared by the following, for example, reaction sequences as depicted in Schemes I, II, IIa, III, IV, V, Va, VI, VII and VIII.

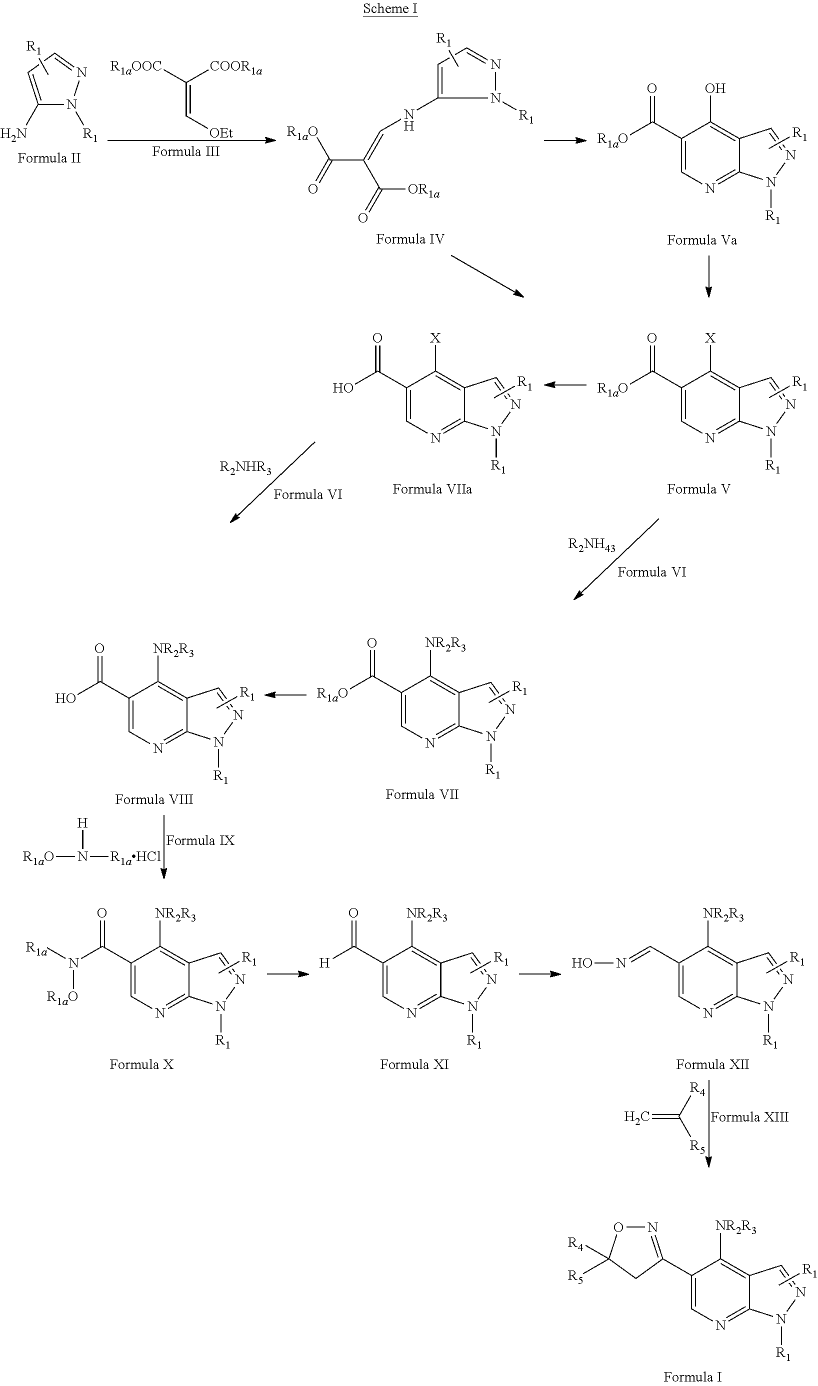

The compounds of Formula I can be prepared by following Scheme I. Thus, compounds of Formula II are reacted with compounds of Formula III to give compounds of Formula IV (wherein R₁ₐ is alkyl), which on heating give compounds of Formula Va, which on reaction with phosphorous oxy halide give compounds of Formula V (wherein X is a halogen) or compounds of Formula IV are reacted with phosphorous oxy halide to give compounds of Formula V (wherein X is same as defined earlier), which are reacted with compounds of Formula VI to give compounds of Formula VII, which on ester hydrolysis give compounds of Formula VIII, or compounds of Formula V on ester hydrolysis give compounds of Formula VIIa, which on reaction with compounds of Formula VI give compounds of Formula VIII, which are reacted with compounds of Formula IX (wherein R₁ₐ is the same as defined earlier) to give compounds of Formula X, which on reduction give compounds of Formula XI, which on reaction with hydroxylamine hydrochloride give compounds of Formula XII, which are finally reacted with compounds of Formula XIII to give compounds of Formula I (wherein R₁, R₂, R₃, R₄ and R₅ are the same as defined earlier).

The compounds of Formula IV can be prepared by the reaction of compounds of Formula II with compounds of Formula III on heating.

The compounds of Formula Va can be prepared by the heating of compounds of Formula IV in one or more solvents, for example, alcohols, for example, methanol, ethanol, propanol or butanol in the presence of a high boiling medium, for example, diphenyl ether, dimethylsulfoxide or mixture(s) thereof.

The compounds of Formula V can be prepared by the reaction of compounds of Formula V a with phosphorous oxy halide on heating.

The compounds of Formula V can be also be prepared by the reaction of compounds of Formula IV with phosphorous oxy halide on heating.

The ester hydrolysis of compounds of Formula V to give compounds of Formula VII a can be carried out in one or more solvents, for example, alcohols, for example, methanol, ethanol, propanol or butanol; ethers, for example, dioxane or tetrahydrofuran; or an alcohol and water mixture.

The ester hydrolysis of compounds of Formula V can be carried out in the presence of one or more inorganic bases, for example, alkali metal hydroxides, for example, potassium hydroxide, sodium hydroxide, lithium hydroxide or mixture(s) thereof.

The reaction of compounds of Formula VIIa with compounds of Formula VI to give compounds of Formula VIII can be carried out in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reaction of compounds of Formula VIIa with compounds of Formula VI can be carried out in the optional presence of one or more bases, for example, triethylamine, pyridine, potassium tert-butoxide, sodium hydride or mixture(s) thereof.

The reaction of compounds of Formula V with compounds of Formula VI to give compounds of Formula VII can be carried out in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reaction of compounds of Formula V with compounds of Formula VI can be carried out in the optional presence of one or more bases, for example, triethylamine, pyridine, potassium tert-butoxide, sodium hydride or mixture(s) thereof.

The ester hydrolysis of compounds of Formula VII to give compounds of Formula VIII can be carried out in one or more solvents, for example, alcohols, for example, methanol, ethanol, propanol or butanol; or an alcohol and water mixture.

The ester hydrolysis of compounds of Formula VII to give compounds of Formula VIII can be carried out in the presence of one or more inorganic bases, for example, alkali metal hydroxides, for example, potassium hydroxide, sodium hydroxide, lithium hydroxide or mixture(s) thereof.

The reaction of compounds of Formula VIII with compounds of Formula IX to give compounds of Formula X can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide; or mixture(s) thereof.

The reaction of compounds of Formula VIII with compounds of Formula IX can be carried out in the presence of one or more bases, for example, N-methylmorpholine; N-ethyldiisopropylamine; 4-dialkylaminopyridines, for example, 4-dimethylaminopyridine; or mixture(s) thereof.

The reduction of compounds of Formula X to give compounds of Formula XI can be carried out in one or more solvents, for example, ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reduction of compounds of Formula X to give compounds of Formula XI can be carried out in the presence of one or more reducing agents, for example, sodium bis(2-methoxyethoxy)aluminum hydride (vitride), lithium aluminium hydride or mixture(s) thereof.

The reaction of compounds of Formula XI with hydroxylamine hydrochloride to give compounds of Formula XII can be carried out in the presence of sodium acetate in one or more solvents, for example, alcohols, for example, methanol, ethanol, propanol, butanol or mixture(s) thereof.

The reaction of compounds of Formula XII with compounds of Formula XIII to give compounds of Formula I can be carried out in the presence of one or more halogenating agents, for example, sodium hypochlorite, N-chlorosuccinimide, N-bromosuccinimide or mixture(s) thereof, in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; halogenated hydrocarbons, for example, dichloromethane, dichloroethane or chloroform; or mixture(s) thereof.

The reaction of compounds of Formula XII with compounds of Formula XIII can be carried out in the optional presence of one or more bases, for example, triethyl amine, trimethyl amine or mixture(s) thereof.

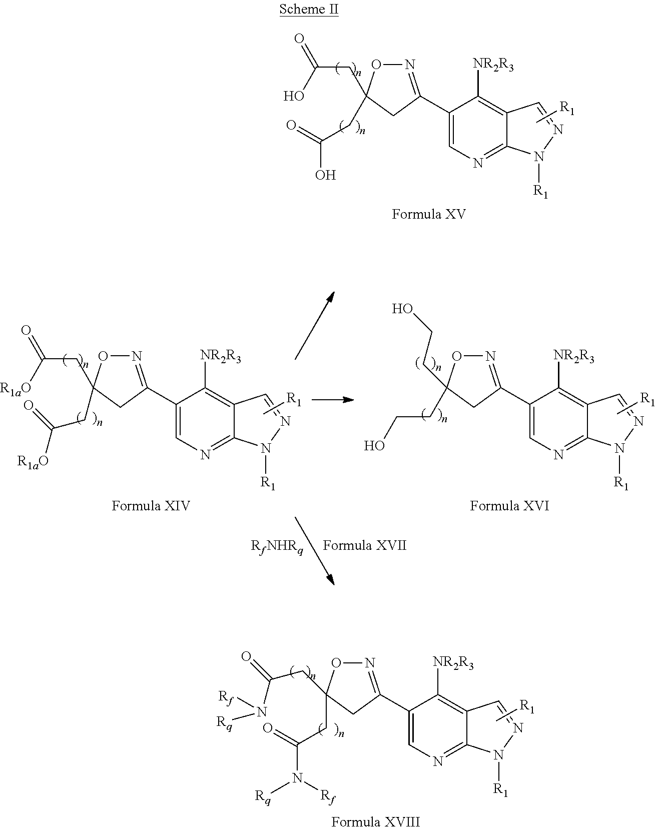

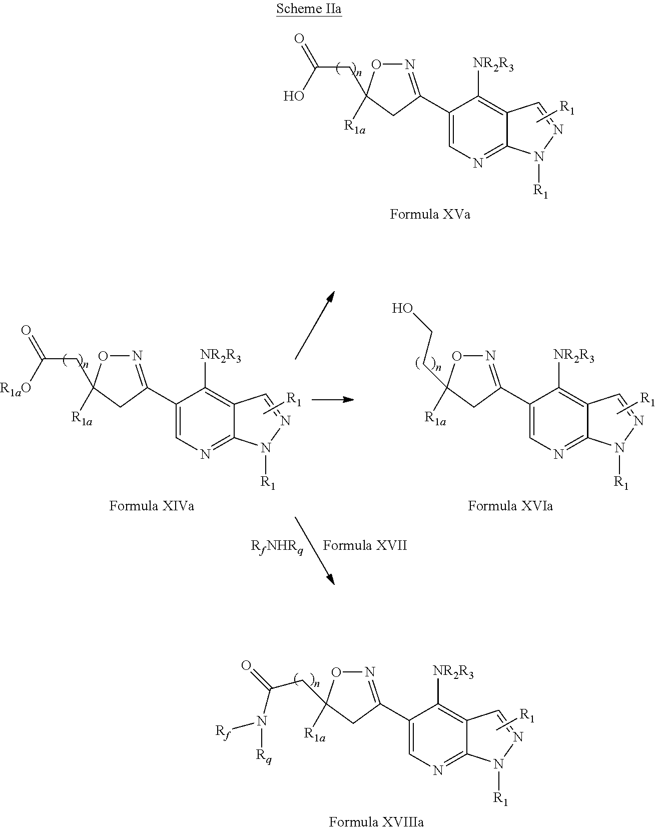

The compounds of Formulae XV, XVa, XVI, XVIa, XVIII and XVIIIa can be prepared by following Schemes II and IIa. Thus,
(i) the ester hydrolysis of compounds of Formula XIV or compounds of Formula XIVa (wherein R₁ₐ is alkyl) gives compounds of Formula XV or compounds of Formula XVa, respectively (wherein n, R₁, R₂ and R₃ are the same as defined earlier);
(ii) the reduction of compounds of Formula XIV or compounds of Formula

XIVa, gives compounds of Formula XVI or compounds of Formula XVIa, respectively (wherein n, R₁, R₂ and R₃ are the same as defined earlier); and
(iii) the reaction of compounds of Formula XIV or compounds of Formula

XIVa, with compounds of Formula XVII gives compounds of Formula XVIII or compounds of Formula XVIIIa, respectively (wherein n, R₁, R₂, R₃, R_{f} and R_{q} are the same as defined earlier).

The ester hydrolysis of compounds of Formula XIV or compounds of Formula XIVa to give compounds of Formula XV or compounds of Formula XVa can be carried out in one or more solvents, for example, alcohols, for example, methanol, ethanol, propanol or butanol; ethers, for example, tetrahydrofuran or diethyl ether; or an ether and water mixture.

The ester hydrolysis of compounds of Formula XIV or compounds of Formula XIVa can be carried out in the presence of one or more inorganic bases, for example, alkali metal hydroxides, for example, potassium hydroxide, sodium hydroxide or lithium hydroxide; alkaline earth metal hydroxides, for example, barium hydroxide octahydrate; or mixture(s) thereof.

The reduction of compounds of Formula XIV or compounds of Formula XIVa to give compounds of Formula XVI or compounds of Formula XVIa can be carried out in one or more solvents, for example, ethers, for example, tetrahydrofuran or diethyl ether; alcohols, for example, methanol, ethanol, propanol or butanol; esters, for example, methyl acetate or ethyl acetate; or mixture(s) thereof.

The reduction of compounds of Formula XIV or compounds of Formula XIVa can be carried out in the presence of one or more reducing agents, for example, sodium borohydride, lithium aluminium hydride, sodium cyanoborohydride, sodium triacetoxyborohydride or mixture(s) thereof.

The reaction of compounds of Formula XIV or compounds of Formula XIVa with compounds of Formula XVII to give compounds of Formula XVIII or compounds of Formula XVIIIa can be carried out in one or more solvents, for example, ethers, for example, tetrahydrofuran or diethyl ether; alcohols, for example, methanol, ethanol, propanol or butanol; or mixture(s) thereof

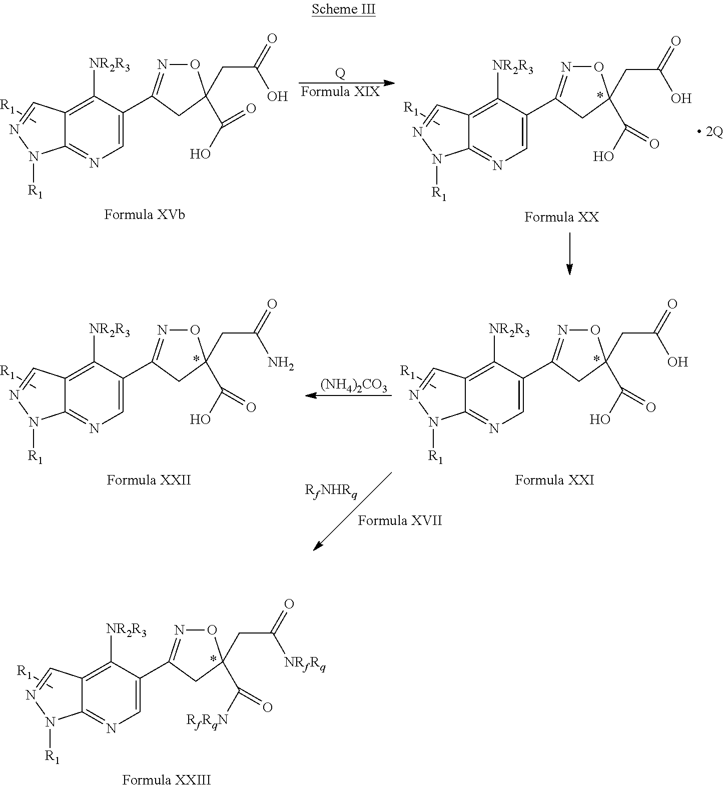

The compounds of Formulae XXI, XXII and XXIII can be prepared by following, Scheme III. Thus, compounds of Formula XVb are reacted with a chiral resolving agent, ‘Q’ of Formula XIX (wherein chiral resolving agent is, for example, L-ephedrine, D-ephedrine, brucine, (1S,2R) (−)-cis-1-amino-2-indanol, (1R,2S) (+)-cis-1-amino-2-indanol, (1R,2R) (−)-1,2-diamino cyclohexane or (1S,2S) (+)-1,2-diamino cyclohexane or α-methylbenzylamine) to give compounds of Formula XX, which on hydrolysis give compounds of Formula XXI, which on reaction with
(i) ammonium carbonate give compounds of Formula XXII (wherein * represents a chiral centre and R₁, R₂ and R₃ are the same as defined earlier).
(ii) compounds of Formula XVII give compounds of Formula XXIII (wherein * represents a chiral centre and R_{f}, R_{q}, R₁, R₂ and R₃ are the same as defined earlier).

The reaction of compounds of Formula XVb with a chiral resolving agent to give compounds of Formula XX can be carried out in one or more solvents, for example, esters, for example, methyl acetate or ethyl acetate; ketones, for example, acetone; nitriles, for example, acetonitrile; or mixture(s) thereof.

The hydrolysis of compounds of Formula XX to give compounds of Formula XXI can be carried out in the presence of one or more inorganic acids, for example, hydrochloric acid or sulphuric acid, in water.

The reaction of compounds of Formula XXI with ammonium carbonate or compounds of Formula XVII to give compounds of Formula XXII or compounds of Formula XXIII, respectively can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide or mixture(s) thereof.

The reaction of compounds of Formula XXI with ammonium carbonate or compounds of Formula XVII can be carried out in the optional presence of one or more bases, for example, triethyl amine, N-ethyldiisopropyl amine or mixture(s) thereof

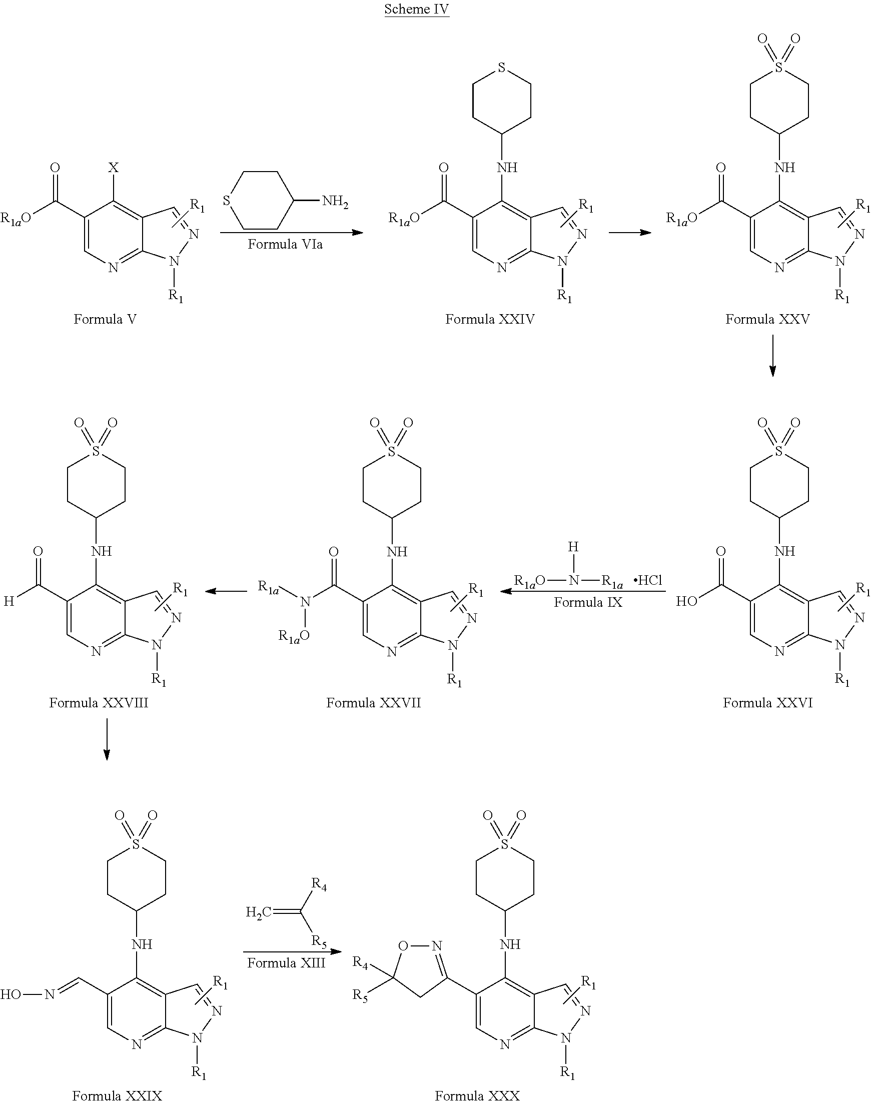

The compounds of Formula XXX can be prepared by following Scheme IV. Thus, compounds of Formula V (wherein X is halogen and R₁ₐ is alkyl) are reacted with compounds of Formula VIa to give compounds of Formula XXIV, which on oxidation give compounds of Formula XXV, which on ester hydrolysis give compounds of Formula XXVI, which on reaction with compounds of Formula IX (wherein R₁ₐ is alkyl) give compounds of Formula XXVII, which on reduction give compounds of Formula XXVIII, which on reaction with hydroxylamine hydrochloride give compounds of Formula XXIX, which are reacted with compounds of Formula XIII to give compounds of Formula XXX (wherein R₁, R₄ and R₅ are the same as defined earlier).

The reaction of compounds of Formula V with compounds of Formula VIa to give compounds of Formula XXIV can be carried out in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reaction of compounds of Formula V with compounds of Formula VIa can be carried out in the optional presence of one or more bases, for example, triethylamine, pyridine, potassium tert-butoxide, sodium hydride or mixture(s) thereof.

The oxidation of compounds of Formula XXIV to give compounds of Formula XXV can be carried out in the presence of one or more oxidizing agents, for example, m-chloroperbenzoic acid, oxone or hydrogen peroxide in one or more solvents, for example, halogenated hydrocarbons, for example, dichloromethane, dichloroethane or chloroform; or mixture(s) thereof.

The ester hydrolysis of compounds of Formula XXV to give compounds of Formula XXVI can be carried out in one or more solvents, for example, alcohols, for example, methanol, ethanol, propanol or butanol; or an alcohol and water mixture.

The ester hydrolysis of compounds of Formula XXV can be carried out in the presence of one or more inorganic bases, for example, alkali metal hydroxides, for example, potassium hydroxide, sodium hydroxide, lithium hydroxide or mixture(s) thereof.

The reaction of compounds of Formula XXVI with compounds of Formula IX to give compounds of Formula XXVII can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide; or mixture(s) thereof.

The reaction of compounds of Formula XXVI with compounds of Formula IX can be carried out in the presence of one or more bases, for example, N-methylmorpholine; N-ethyldiisopropylamine; 4-dialkylaminopyridines, for example, 4-dimethylaminopyridine; or mixture(s) thereof.

The reduction of compounds of Formula XXVII to give compounds of Formula XXVIII can be carried out in one or more solvents, for example, ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reduction of compounds of Formula XXVII can be carried out in the presence of one or more reducing agents, for example, sodium bis(2-methoxyethoxy) aluminum hydride (vitride), lithium aluminium hydride or mixture(s) thereof.

The reaction of compounds of Formula XXVIII with hydroxylamine hydrochloride to give compounds of Formula XXIX can be carried out in the presence of sodium acetate in one or more solvents, for example, alcohols, for example, methanol, ethanol, propanol, butanol or mixture(s) thereof.

The reaction of compounds of Formula XXIX with compounds of Formula XIII to give compounds of Formula XXX can be carried out in the presence of one or more halogenating agents, for example, sodium hypochlorite, N-chlorosuccinimide, N-bromosuccinimide or mixture(s) thereof, in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; halogenated hydrocarbons, for example, dichloromethane, dichloroethane or chloroform; or mixture(s) thereof.

The reaction of compounds of Formula XXIX with compounds of Formula XIII can be carried out in the optional presence of one or more bases, for example, triethyl amine, trimethyl amine or mixture(s) thereof.

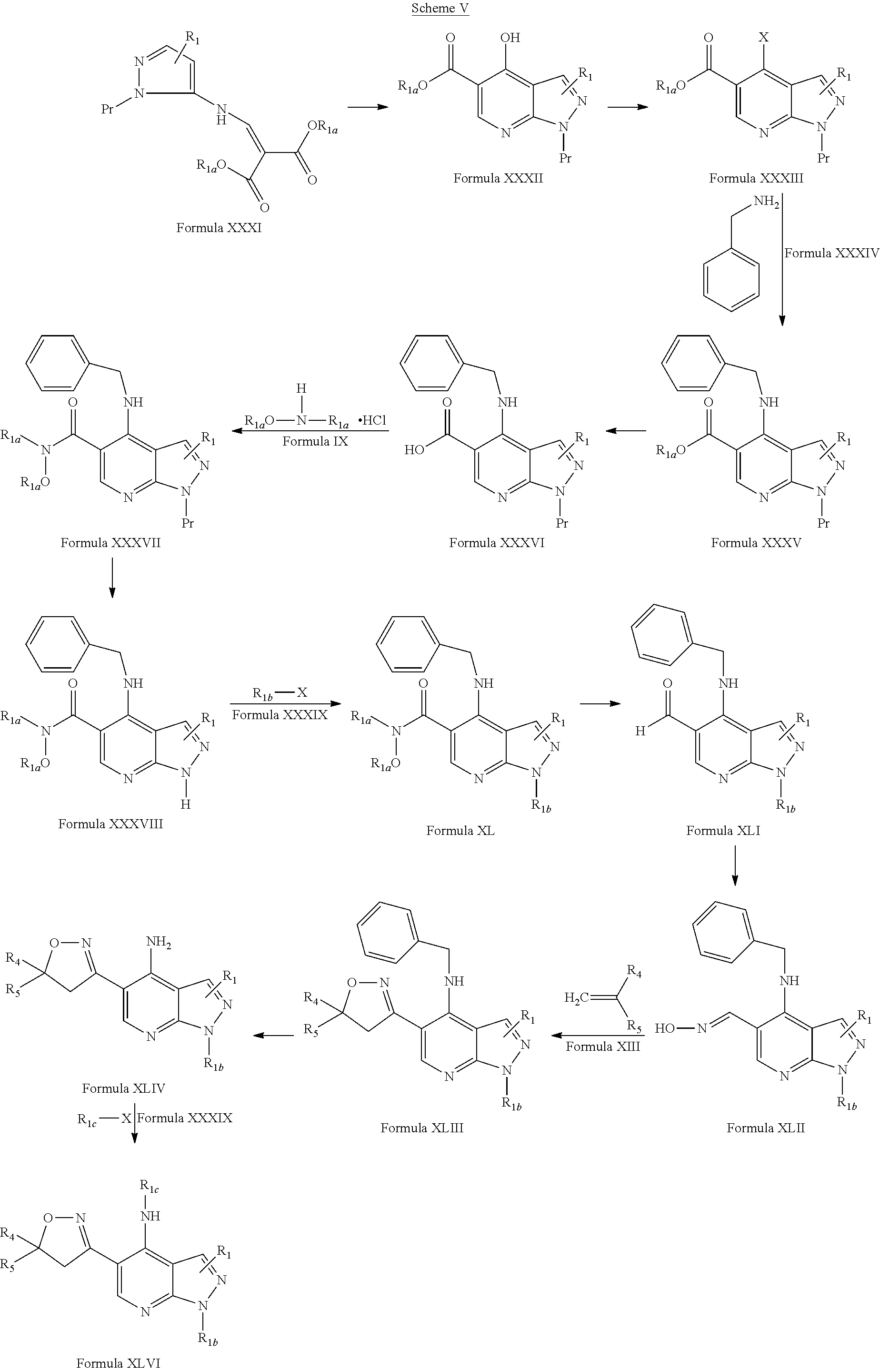

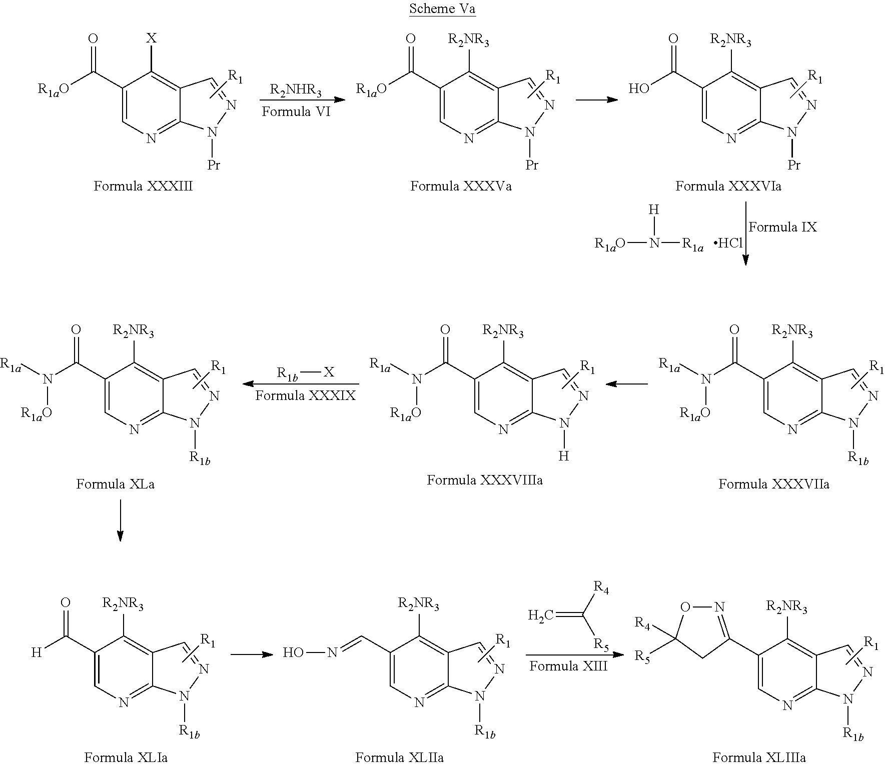

The compounds of Formula XLVI can be prepared by following Scheme V. Thus, compounds of Formula XXXI (wherein R₁ₐ is alkyl and Pr is a protecting group, for example, p-methoxy benzyl or benzyl) on heating give compounds of Formula XXXII, which on reaction with phosphorous oxy halide give compounds of Formula XXXIII (wherein X is a halogen), which on reaction with compounds of Formula XXXIV give compounds of Formula XXXV, which on ester hydrolysis give compounds of Formula XXXVI, which on reaction with compounds of Formula IX (wherein R₁ₐ is the same as defined earlier) give compounds of Formula XXXVII, which on deprotection give compounds of Formula XXXVIII, which on reaction with compounds of Formula XXXIX (wherein X is halogen) give compounds of Formula XL, which on reduction give compounds of Formula XLI, which on reaction with hydroxylamine hydrochloride give compounds of Formula XLII, which on reaction with compounds of Formula XIII give compounds of Formula XLIII, which on deprotection give compounds of Formula XLIV, which are finally reacted with compounds of Formula XLV (wherein X is halogen) to give compounds of Formula XLVI (wherein R_{1b} is alkyl or cycloalkyl, R_{1c} is aryl or heteroaryl and R₁, R₄ and R₅ are the same as defined earlier).

The compounds of Formula XLIIIa can be prepared by following Scheme Va. Thus, compounds of Formula XXXIII (wherein X is halogen, R₁ₐ is alkyl and Pr is a protecting group, for example, p-methoxy benzyl or benzyl) on reaction with compounds of Formula VI give compounds of Formula XXXVa, which on ester hydrolysis give compounds of Formula XXXVIa, which on reaction with compounds of Formula IX (wherein R₁ₐ is the same as defined earlier) give compounds of Formula XXXVIIa, which on deprotection give compounds of Formula XXXVIIIa, which on reaction with compounds of Formula XXXIX (wherein X is halogen) give compounds of Formula XLa, which on reduction give compounds of Formula XLIa, which on reaction with hydroxylamine hydrochloride give compounds of Formula XLIIa, which are finally reacted with compounds of Formula XIII to give compounds of Formula XLIIIa (wherein R_{1b} is alkyl or cycloalkyl and R₁, R₂, R₃, R₄ and R₅ are the same as defined earlier).

The compounds of Formula XXXII can be prepared by the heating of compounds of Formula XXXI in one or more solvents, for example, alcohols, for example, methanol, ethanol, propanol or butanol in the presence of a high boiling medium, for example, diphenyl ether, dimethylsulfoxide or mixture(s) thereof.

The compounds of Formula XXXIII can be prepared by the reaction of compounds of XXXII with phosphorous oxy halide on heating.

The reaction of compounds of Formula XXXIII with compounds of Formula XXXIV or compounds of Formula VI to give compounds of Formula XXXV or compounds of Formula XXXVa, respectively can be carried out in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reaction of compounds of Formula XXXIII with compounds of Formula XXXIV or compounds of Formula VI can be carried out in the optional presence of one or more bases, for example, triethylamine, pyridine, potassium tert-butoxide, sodium hydride or mixture(s) thereof.

The ester hydrolysis of compounds of Formula XXXV or compounds of Formula XXXVa to give compounds of Formula XXXVI or compounds of Formula XXXVIa, respectively can be carried out in one or more solvents, for example, alcohols, for example, methanol, ethanol, propanol or butanol; or an alcohol and water mixture.

The ester hydrolysis of compounds of Formula XXXV or compounds of Formula XXXVa can be carried out in the presence of one or more inorganic bases, for example, alkali metal hydroxides, for example, potassium hydroxide, sodium hydroxide, lithium hydroxide or mixture(s) thereof.

The reaction of compounds of Formula XXXVI or compounds of Formula XXXVIa with compounds of Formula IX to give compounds of Formula XXXVII or compounds of Formula XXXVIIa, respectively can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide; or mixture(s) thereof.

The reaction of compounds of Formula XXXVI or compounds of Formula XXXVIa with compounds of Formula IX can be carried out in the presence of one or more bases, for example, N-methylmorpholine; N-ethyldiisopropylamine; 4-dialkylaminopyridines, for example, 4-dimethylaminopyridine; or mixture(s) thereof.

The deprotection of compounds of Formula XXXVII or compounds of Formula XXXVIIa to give compounds of Formula XXXVIII or compounds of Formula XXXVIIIa, respectively can be carried out in the presence of one or more acids, for example, hydrochloric acid, trifluoroacetic acid, p-toluene sulphonic acid or mixture(s) thereof in one or more solvents, for example, alcohols, for example, methanol, ethanol, propanol or butanol; halogenated hydrocarbons, for example, dichloromethane, dichloroethane or chloroform; or mixture(s) thereof.

The reaction of compounds of Formula XXXVIII or compounds of Formula XXXVIIIa with compounds of Formula XXXIX to give compounds of Formula XL or compounds of Formula XLa, respectively can be carried out in the presence of one or more inorganic bases, for example, alkali metal carbonates, for example, sodium carbonate or potassium carbonate, alkali metal hydrides, for example, sodium hydride or mixture(s) thereof or one or more organic bases, for example, triethyl amine, N-ethyldiisopropyl amine or mixture(s) thereof in one or more solvents, for example, nitriles, for example, acetonitrile; halogenated hydrocarbons, for example, dichloromethane, dichloroethane or chloroform; amides, for example, dimethylformamide or dimethylacetamide; or mixture(s) thereof.

The reduction of compounds of Formula XL or compounds of Formula XLa to give compounds of Formula XLI or compounds of Formula XLIa, respectively can be carried out in one or more solvents, for example, ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reduction of compounds of Formula XL or compounds of Formula XLa can be carried out in the presence of one or more reducing agents, for example, sodium bis(2-methoxyethoxy)aluminum hydride (vitride), lithium aluminium hydride or mixture(s) thereof.

The reaction of compounds of Formula XLI or compounds of Formula XLIa with hydroxylamine hydrochloride to give compounds of Formula XLII or compounds of Formula XLIIa, respectively can be carried out in the presence of sodium acetate in one or more solvents, for example, alcohols, for example, methanol, ethanol, propanol, butanol or mixture(s) thereof.

The reaction of compounds of Formula XLII or compounds of Formula XLIIa with compounds of Formula XIII to give compounds of Formula XLIII or compounds of Formula XLIIIa, respectively can be carried out in the presence of one or more halogenating agents, for example, sodium hypochlorite, N-chlorosuccinimide, N-bromosuccinimide or mixture(s) thereof, in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; halogenated hydrocarbons, for example, dichloromethane, dichloroethane or chloroform; or mixture(s) thereof.

The reaction of compounds of Formula XLII or compounds of Formula XLIIa with compounds of Formula XIII can be carried out in the optional presence of one or more bases, for example, triethyl amine, trimethyl amine or mixture(s) thereof.

The deprotection of compounds of Formula XLIII to give compounds of Formula XLIV can be carried out in the presence of palladium on carbon/hydrogen, palladium hydroxide/carbon with hydrogen, ammonium formate/palladium on carbon, in one or more solvents, for example, alcohols, for example, methanol, ethanol, propanol or butanol; halogenated hydrocarbons, for example, dichloromethane, dichloroethane or chloroform; or mixture(s) thereof.

The reaction of compounds of Formula XLIV with compounds of Formula XLV to give compounds of Formula XLVI can be carried out in the presence of one or more transition metal catalysts, for example, tris(dibenzylidineacetone)dipalladium(0), palladium(II) acetate, tetrakis(triphenylphosphine)palladium(0), tetrakis (methyldiphenylphosphine) palladium(0), trans-dichlorobis(methyldiphenylphosphine) palladium(II), dichlorobis (triphenylphosphine)palladium(II), bis[1,2-bis(diphenylphosphino) ethane]palladium(0), copper (I) iodide, cuprous oxide, cuprous bromide, cuprous chloride or mixture(s) thereof.

The reaction of compounds of Formula XLIV with compounds of Formula XLV can be carried out in the presence of one or more phosphine ligands, for example, xantphos, 1,1′-bis(di-tert-butylphosphino)ferrocene, 2,2′-bis(diphenylphosphino)diphenyl ether (DPEphos), bis(triethylphosphine)nickel (II) chloride, (R,S)-2,2′-bis(diphenylphosphino)-1,1′-binaphthyl, (S)-2,2′-bis(diphenylphosphino)-1,1′-binaphthyl, (R)-2,2′-bis(diphenylphosphino)-1,1′-binaphthyl or mixture(s) thereof.

The reaction of compounds of Formula XLIV with compounds of Formula XLV can be carried out in the presence of one or more bases, for example, amines, for example, N-ethyldiisopropylamine, triethyl amine or dimethylamino pyridine, alkali metal alkoxides, for example, sodium tert-butoxide, potassium tert-butoxide, sodium methoxide, lithium methoxide, potassium methoxide or cesium methoxide, alkali metal hydroxides, for example, sodium hydroxide, lithium hydroxide, potassium hydroxide or cesium hydroxide, alkali metal halides, for example, potassium fluoride, alkali metal carbonates, for example, sodium carbonate, potassium carbonate or cesium carbonate or mixture(s) thereof.

The reaction of compounds of Formula XLIV with compounds of Formula XLV can be carried out in one or more solvents, for example, ethers, for example, dioxane or tetrahydrofuran, amides, for example, dimethylformamide or dimethylacetamide; sulfoxides, for example, dimethylsulfoxide; hydrocarbons, for example, hexane or toluene; or mixture(s) thereof

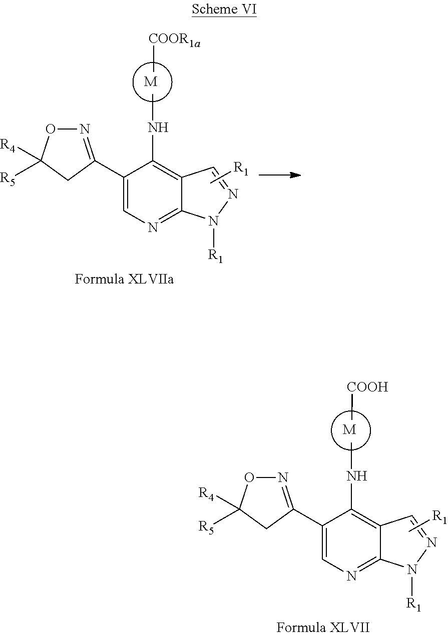

The compounds of Formula XLVII can be prepared by following Scheme VI. Thus, ester hydrolysis of compounds of Formula XLVIIa (wherein R₁ₐ is alkyl) gives compounds of Formula XLVII (wherein R₁, R₄ and R₅ are the same as defined earlier and ring M is cyclobutyl or cyclohexyl ring).

The ester hydrolysis of compounds of Formula XLVIIa to give compounds of Formula XLVII can be carried out in the presence of one or more acids, for example, hydrochloric acid, trifluoroacetic acid, p-toluene sulphonic acid or mixture(s) thereof in one or more solvents, for example, halogenated hydrocarbons, for example, dichloromethane, dichloroethane, chloroform, or mixture(s) thereof

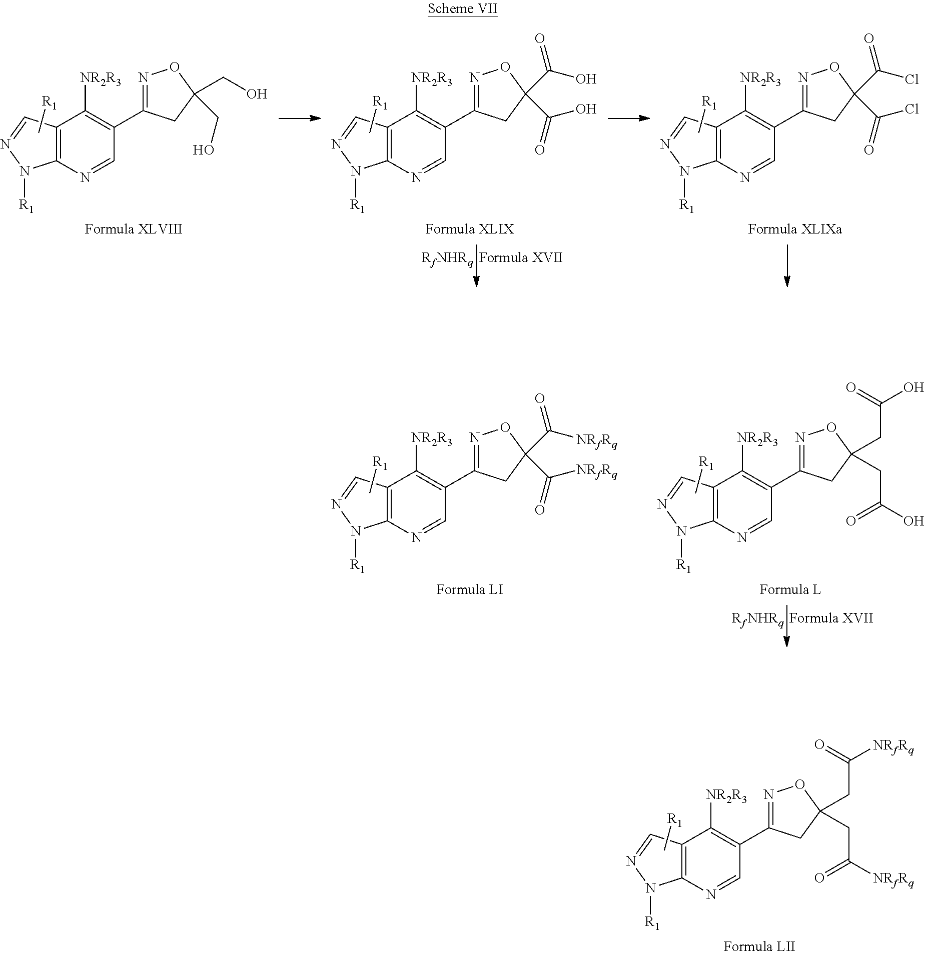

The compounds of Formulae XLIX, L, LI and LII can be prepared by following Scheme VII. Thus, compounds of Formula XLVIII are oxidized to give compounds of Formula XLIX, which on
(i) reaction with compounds of Formula XVII give compounds of Formula LI (wherein R₁, R₂, R₃, R_{f} and R_{q} are the same as defined earlier).
(ii) halogenation give compounds of Formula XLIXa, which on one carbon homologation give compounds of Formula L, which are, finally reacted with compounds of Formula XVII to give compounds of Formula LII (wherein R₁, R₂, R₃, R_{f} and R_{q} are the same as defined earlier).

The oxidation of compounds of Formula XLVIII to give compounds of Formula XLIX can be carried out in the presence of one or more oxidizing agents, for example, potassium permanganate, Jone's reagent or potassium dichromate in one or more solvents, for example, water; ketones, for example, acetone; ethers, for example, dioxane, diethyl ether or tetrahydrofuran; or mixture(s) thereof.

The reaction of compounds of Formula XLIX with compounds of Formula XVII to give compounds of Formula LI can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide; or mixture(s) thereof.

The reaction of compounds of Formula XLIX with compounds of Formula XVII can be carried out in the optional presence of one or more bases, for example, triethyl amine, N-ethyldiisopropyl amine or mixture(s) thereof.

The halogenation of compounds of Formula XLIX to give compounds of Formula XLIXa can be carried out in the presence of one or more halogenating agents, for example, phosphorous pentachloride, phosphohorous pentabromide, phosphorous trichloride, phosphorous tribromide, thionyl chloride, oxalyl chloride or mixture(s) thereof in one or more solvents, for example, amides, for example, dimethylformamide or dimethylacetamide; sulfoxides, for example, dimethylsulphoxide; or mixture(s) thereof.

The one carbon homologation of compounds of Formula XLIX a to give compounds of Formula L can be carried out in the presence of reagents, for example, trimethylsilyldiazomethane or diazomethane in one or more solvents, for example, amides, for example, dimethylformamide or dimethylacetamide; sulfoxides, for example, dimethylsulphoxide; ethers, for example, tetrahydrofuran, dioxane or diethyl ether; nitriles, for example, acetonitrile; hydrocarbons, for example, hexane or toluene; alcohols, for example, methanol, ethanol, propanol or butanol; or mixture(s) thereof.

The one carbon homologation of compounds of Formula XLIXa can be carried out in the presence of one or more organic bases, for example, trimethylamine, triethylamine, tribenzylamine, N-ethyldiisopropylamine or mixture(s) thereof.

The one carbon homologation can be carried out by the reaction of compounds of Formula XLIXa with water in one or more solvents, for example, ethers, for example, tetrahydrofuran, dioxane, diethyl ether or mixture(s) thereof.

The one carbon homologation of compounds of Formula XLIXa can be carried out in the presence of one or more catalysts, for example, silver oxide, copper or platinum.

The reaction of compounds of Formula L with compounds of Formula XVII to give compounds of Formula LII can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide or mixture(s) thereof.

The reaction of compounds of Formula L with compounds of Formula XVII can be carried out in the optional presence of one or more bases, for example, triethyl amine, N-ethyldiisopropyl amine or mixture(s) thereof.

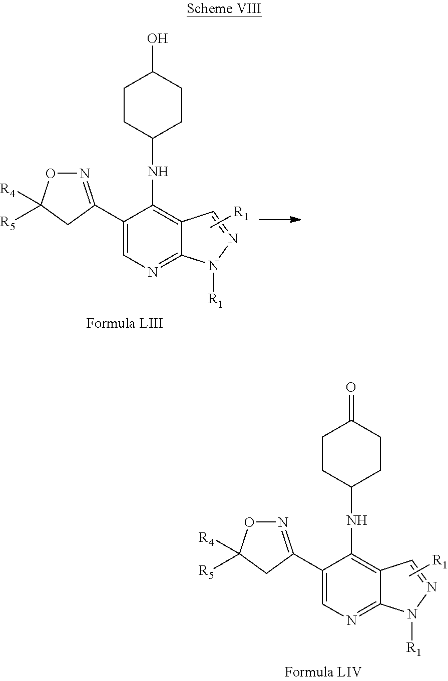

The compounds of Formula LIV can be prepared by following Scheme VIII. Thus, compounds of Formula LIII are oxidized to give compounds of Formula LIV (wherein R₁, R₄ and R₅ are the same as defined earlier).

The oxidation of compounds of Formula LIII to give compounds of Formula LIV can be carried out in the presence of one or more oxidizing agents, for example, pyridinium chlorochromate, pyridinium dichromate, dess martin periodinane or mixture(s) thereof, in one or more solvents, for example, halogenated hydrocarbons, for example, dichloromethane, dichloroethane or chloroform; ethers, for example, tetrahydrofuran or diethyl ether; amides, for example, dimethylformamide or dimethylacetamide; sulfoxides, for example, dimethylsulfoxide, or mixture(s) thereof.

The compounds of Formula Ia can be prepared by following the methods disclosed in WO 2007/031838.

In the above schemes, where the specific solvents, bases, reducing agents, activating reagents, coupling reagents, halogenating agents, chiral resolving agents, acids, oxidizing agents, transition metal catalysts, phosphine ligands etc., are mentioned, it is to be understood that other solvents, bases, reducing agents, activating reagents, coupling reagents, halogenating agents, chiral resolving agents, acids, oxidizing agents, transition metal catalysts, phosphine ligands etc., known to those skilled in the art may be used. Similarly, reaction parameters such as the reaction temperature and duration may be adjusted according to the desired needs.

An illustrative list of compounds of the invention includes these listed below:
{3-[4-(Cyclohexylamino)-1-ethyl-1H-pyrazolo[3,4-b]pyridin-5-yl]-4,5-dihydroisoxazole-5,5-diyl}dimethanol (Compound No. 1);
Methyl 3-[4-(cyclohexylamino)-1-ethyl-1H-pyrazolo[3,4-b]pyridin-5-yl]-5-(2-methoxy-2-oxoethyl)-4,5-dihydroisoxazole-5-carboxylate (Compound No. 2);
Methyl 3-[1-ethyl-4-(tetrahydro-2H-pyran-4-ylamino)-1H-pyrazolo[3,4-b]pyridin-5-yl]-5-(2-methoxy-2-oxoethyl)-4,5-dihydroisoxazole-5-carboxylate (Compound No. 3);
{3-[1-Ethyl-4-(tetrahydro-2H-pyran-4-ylamino)-1H-pyrazolo[3,4-b]pyridin-5-yl]-4,5-dihydroisoxazole-5,5-diyl}dimethanol (Compound No. 4);
4-({5-[5,5-Bis(hydroxymethyl)-4,5-dihydroisoxazol-3-yl]-1-ethyl-1H-pyrazolo[3,4-b]pyridin-4-yl}amino)cyclohexanol (Compound No. 5);
5-(Carboxymethyl)-3-[4-(cyclohexylamino)-1-ethyl-1H-pyrazolo[3,4-b]pyridin-5-yl]-4,5-dihydroisoxazole-5-carboxylic acid (Compound No. 6);
2-{3-[4-(Cyclohexylamino)-1-ethyl-1H-pyrazolo[3,4-b]pyridin-5-yl]-5-(hydroxymethyl)-4,5-dihydroisoxazol-5-yl}ethanol (Compound No. 7);
5-(2-Amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 8);
3-[4-(Cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 9);
Methyl 3-[4-(cyclopropylamino)-1-ethyl-1H-pyrazolo[3,4-b]pyridin-5-yl]-5-methyl-4,5-dihydroisoxazole-5-carboxylate (Compound No. 10);
3-[4-(Cyclopropylamino)-1-ethyl-1H-pyrazolo[3,4-b]pyridin-5-yl]-5-methyl-4,5-dihydroisoxazole-5-carbonitrile (Compound No. 11);
Methyl 3-[4-(cyclopropylamino)-1-ethyl-1H-pyrazolo[3,4-b]pyridin-5-yl]-5-(2-methoxy-2-oxoethyl)-4,5-dihydroisoxazole-5-carboxylate (Compound No. 12);
Methyl 3-[4-(cyclopentylamino)-1,3-dimethyl-1H-pyrazolo[3,4-b]pyridin-5-yl]-5-methyl-4,5-dihydroisoxazole-5-carboxylate (Compound No. 13);
Methyl 3-[4-(cyclopentylamino)-1,3-dimethyl-1H-pyrazolo[3,4-b]pyridin-5-yl]-5-(2-methoxy-2-oxoethyl)-4,5-dihydroisoxazole-5-carboxylate (Compound No. 14);
Methyl 3-[4-(cyclopropylamino)-1,3-dimethyl-1H-pyrazolo[3,4-b]pyridin-5-yl]-5-(2-methoxy-2-oxoethyl)-4,5-dihydroisoxazole-5-carboxylate (Compound No. 15);
Methyl 3-[4-(cyclopentylamino)-1-ethyl-1H-pyrazolo[3,4-b]pyridin-5-yl]-5-methyl-4,5-dihydroisoxazole-5-carboxylate (Compound No. 16);
Methyl 3-[4-(cyclopropylamino)-1,3-dimethyl-1H-pyrazolo[3,4-b]pyridin-5-yl]-5-methyl-4,5-dihydroisoxazole-5-carboxylate (Compound No. 17);
Methyl 3-[4-(cyclopentylamino)-1-ethyl-1H-pyrazolo[3,4-b]pyridin-5-yl]-5-(2-methoxy-2-oxoethyl)-4,5-dihydroisoxazole-5-carboxylate (Compound No. 18);
5-(Carboxymethyl)-3-[4-(cyclopropylamino)-1-ethyl-1H-pyrazolo[3,4-b]pyridin-5-yl]-4,5-dihydroisoxazole-5-carboxylic acid (Compound No. 19);
3-[4-(Cyclopropylamino)-1-ethyl-1H-pyrazolo[3,4-b]pyridin-5-yl]-5-methyl-4,5-dihydroisoxazole-5-carboxylic acid (Compound No. 20);
{3-[4-(Cyclopropylamino)-1-ethyl-1H-pyrazolo[3,4-b]pyridin-5-yl]-5-methyl-4,5-dihydroisoxazol-5-yl}methanol (Compound No. 21);

2-[3-[4-(Cyclopropylamino)-1-ethyl-1H-pyrazolo[3,4-b]pyridin-5-yl]-5-(hydroxymethyl)-4,5-dihydroisoxazol-5-yl]ethanol (Compound No. 22);
{3-[4-(Cyclopentylamino)-1,3-dimethyl-1H-pyrazolo[3,4-b]pyridin-5-yl]-5-methyl-4,5-dihydroisoxazol-5-yl}methanol (Compound No. 23);

2-[3-[4-(Cyclopentylamino)-1,3-dimethyl-1H-pyrazolo[3,4-b]pyridin-5-yl]-5-(hydroxymethyl)-4,5-dihydroisoxazol-5-yl]ethanol (Compound No. 24);
{3-[4-(Cyclopropylamino)-1,3-dimethyl-1H-pyrazolo[3,4-b]pyridin-5-yl]-5-methyl-4,5-dihydroisoxazol-5-yl}methanol (Compound No. 25);
{3-[4-(Cyclopentylamino)-1-ethyl-1H-pyrazolo[3,4-b]pyridin-5-yl]-5-methyl-4,5-dihydroisoxazol-5-yl}methanol (Compound No. 26);
5-(2-Amino-2-oxoethyl)-3-[4-(cyclopropylamino)-1-ethyl-1H-pyrazolo[3,4-b]pyridin-5-yl]-4,5-dihydroisoxazole-5-carboxamide (Compound No. 27);
3-[4-(Cyclopropylamino)-1-ethyl-1H-pyrazolo[3,4-b]pyridin-5-yl]-5-methyl-4,5-dihydroisoxazole-5-carboxamide (Compound No. 28);
3-[4-(Cyclopentylamino)-1,3-dimethyl-1H-pyrazolo[3,4-b]pyridin-5-yl]-N,5-dimethyl-4,5-dihydroisoxazole-5-carboxamide (Compound No. 29);
3-[4-(Cyclopentylamino)-1,3-dimethyl-1H-pyrazolo[3,4-b]pyridin-5-yl]-5-methyl-4,5-dihydroisoxazole-5-carboxamide (Compound No. 30);
5-(2-Amino-2-oxoethyl)-3-[4-(cyclopentylamino)-1,3-dimethyl-1H-pyrazolo[3,4-b]pyridin-5-yl]-4,5-dihydroisoxazole-5-carboxamide (Compound No. 31);
N-cyclopropyl-3-[4-(cyclopropylamino)-1,3-dimethyl-1H-pyrazolo[3,4-b]pyridin-5-yl]-5-[2-(cyclopropylamino)-2-oxoethyl]-4,5-dihydroisoxazole-5-carboxamide (Compound No. 32);
5-(2-Amino-2-oxoethyl)-3-[4-(cyclopropylamino)-1,3-dimethyl-1H-pyrazolo[3,4-b]pyridin-5-yl]-4,5-dihydroisoxazole-5-carboxamide (Compound No. 33);

(5S^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 34);

(5R^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 35);

(5S^{$})-5-(2-amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 36);

(5R^{$})-5-(2-amino-2-oxo ethyl)-3-[4-(cyclohexylamino)-1-ethyl-1H-pyrazolo[3,4-b]pyridin-5-yl]-4,5-dihydroisoxazole-5-carboxamide (Compound No. 37);

(5S^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 38);

(5R^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 39);
5-(2-Amino-2-oxoethyl)-3-{4-[(1,1-dioxidotetrahydro-2H-thiopyran-4-yl)amino]-1-ethyl-1H-pyrazolo [3,4-b]pyridin-5-yl}-4,5-dihydroisoxazole-5-carboxamide (Compound No. 40);
(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)dimethanol (Compound No. 41);
4-({5-[5,5-Bis(hydroxymethyl)-4,5-dihydroisoxazol-3-yl]-1-ethyl-1H-pyrazolo[3,4-b]pyridin-4-yl}amino)cyclohexanecarboxylic acid (Compound No. 42);
4-({5-[5,5-Bis(hydroxymethyl)-4,5-dihydroisoxazol-3-yl]-1-ethyl-3-methyl-1H-pyrazolo[3,4-b]pyridin-4-yl}amino)cyclohexanecarboxylic acid (Compound No. 43);
5-(2-Amino-2-oxo ethyl)-3-{4-[(1,1-dioxidotetrahydro-2H-thiopyran-4-yl)amino]-1-ethyl-3-methyl-1H-pyrazolo[3,4-b]pyridin-5-yl}-4,5-dihydroisoxazole-5-carboxamide (Compound No. 44);
3-{4-[(1,1-Dioxidotetrahydro-2H-thiopyran-4-yl)amino]-1-ethyl-1H-pyrazolo[3,4-b]pyridin-5-yl}-N-methyl-5-[2-(methylamino)-2-oxoethyl]-4,5-dihydroisoxazole-5-carboxamide (Compound No. 45);
3-{4-[(1,1-Dioxidotetrahydro-2H-thiopyran-4-yl)amino]-1-ethyl-3-methyl-1H-pyrazolo[3,4-b]pyridin-5-yl}-N-methyl-5-[2-(methylamino)-2-oxo ethyl]-4,5-dihydroisoxazole-5-carboxamide (Compound No. 46);
(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)dimethanol (Compound No. 47);
{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl} dimethanol (Compound No. 48);
3-({5-[5,5-Bis(hydroxymethyl)-4,5-dihydroisoxazol-3-yl]-1-ethyl-1H-pyrazolo[3,4-b]pyridin-4-yl}amino)cyclobutanecarboxylic acid (Compound No. 49);
3-({5-[5,5-Bis(hydroxymethyl)-4,5-dihydroisoxazol-3-yl]-1-ethyl-3-methyl-1H-pyrazolo[3,4-b]pyridin-4-yl}amino)cyclobutanecarboxylic acid (Compound No. 50);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)diacetamide (Compound No. 51);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-methylacetamide) (Compound No. 52);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-methylacetamide) (Compound No. 53);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)diacetamide (Compound No. 54);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-ethylacetamide) (Compound No. 55);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-ethylacetamide) (Compound No. 56);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}diacetamide (Compound No. 57);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}diacetamide (Compound No. 58);
2,2′-{3-[4-(Cyclohexylamino)-1-ethyl-3-methyl-1H-pyrazolo[3,4-b]pyridin-5-yl]-4,5-dihydroisoxazole-5,5-diyl}diacetamide (Compound No. 59);
2,2′-{3-[4-(Cyclohexylamino)-1-ethyl-1H-pyrazolo[3,4-b]pyridin-5-yl]-4,5-dihydroisoxazole-5,5-diyl}diacetamide (Compound No. 60);
2,2′-{3-[4-(Cyclohexylamino)-1-ethyl-1H-pyrazolo[3,4-b]pyridin-5-yl]-4,5-dihydroisoxazole-5,5-diyl}bis(N-methylacetamide) (Compound No. 61);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 62);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 63);
2,2′-{3-[4-(Cyclohexylamino)-1-ethyl-3-methyl-1H-pyrazolo[3,4-b]pyridin-5-yl]-4,5-dihydroisoxazole-5,5-diyl}bis(N-methylacetamide) (Compound No. 64);
5-(2-Amino-2-oxo ethyl)-3-[1-ethyl-3-methyl-4-(tetrahydro-2H-pyran-4-ylamino)-1H-pyrazolo[3,4-b]pyridin-5-yl]-4,5-dihydroisoxazole-5-carboxamide (Compound No. 65);
3-[1-Ethyl-3-methyl-4-(tetrahydro-2H-pyran-4-ylamino)-1H-pyrazolo[3,4-b]pyridin-5-yl]-N-methyl-5-[2-(methylamino)-2-oxo ethyl]-4,5-dihydroisoxazole-5-carboxamide (Compound No. 66);
5-(2-Amino-2-oxo ethyl)-3-[1-ethyl-4-(tetrahydro-2H-pyran-4-ylamino)-1H-pyrazolo[3,4-b]pyridin-5-yl]-4,5-dihydroisoxazole-5-carboxamide (Compound No. 67);
3-[1-Ethyl-4-(tetrahydro-2H-pyran-4-ylamino)-1H-pyrazolo[3,4-b]pyridin-5-yl]-N-methyl-5-[2-(methylamino)-2-oxo ethyl]-4,5-dihydroisoxazole-5-carboxamide (Compound No. 68);
5-(2-Amino-2-oxo ethyl)-3-[4-(cyclohexylamino)-1-ethyl-3-methyl-1H-pyrazolo[3,4-b]pyridin-5-yl]-4,5-dihydroisoxazole-5-c arbox amide (Compound No. 69);
3-[4-(Cyclohexylamino)-1-ethyl-3-methyl-1H-pyrazolo[3,4-b]pyridin-5-yl]-N-methyl-5-[2-(methylamino)-2-oxo ethyl]-4,5-dihydroisoxazole-5-carboxamide (Compound No. 70);
4-({5-[5,5-Bis(2-amino-2-oxo ethyl)-4,5-dihydroisoxazol-3-yl]-1-ethyl-1H-pyrazolo[3,4-b]pyridin-4-yl}amino)cyclohexanecarboxylic acid (Compound No. 71);
4-({5-[5,5-Bis(2-amino-2-oxoethyl)-4,5-dihydroisoxazol-3-yl]-1-ethyl-3-methyl-1H-pyrazolo[3,4-b]pyridin-4-yl}amino)cyclohexanecarboxylic acid (Compound No. 72);
4-[(5-{5,5-Bis[2-(methylamino)-2-oxoethyl]-4,5-dihydroisoxazol-3-yl}-1-ethyl-3-methyl-1H-pyrazolo[3,4-b]pyridin-4-yl)amino]cyclohexanecarboxylic acid (Compound No. 73);
4-[(5-{5,5-Bis[2-(methylamino)-2-oxoethyl]-4,5-dihydroisoxazol-3-yl}-1-ethyl-1H-pyrazolo[3,4-b]pyridin-4-yl)amino]cyclohexanecarboxylic acid (Compound No. 74);
4-[(5-{5,5-Bis[2-(ethylamino)-2-oxoethyl]-4,5-dihydroisoxazol-3-yl}-1-ethyl-1H-pyrazolo[3,4-b]pyridin-4-yl)amino]cyclohexanecarboxylic acid (Compound No. 75);
4-[(5-{5,5-Bis[2-(ethylamino)-2-oxoethyl]-4,5-dihydroisoxazol-3-yl}-1-ethyl-3-methyl-1H-pyrazolo[3,4-b]pyridin-4-yl)amino]cyclohexanecarboxylic acid (Compound No. 76);
4-[(1-Ethyl-5-{5-[2-(ethylamino)-2-oxoethyl]-5-(ethylcarbamoyl)-4,5-dihydroisoxazol-3-yl}-3-methyl-1H-pyrazolo[3,4-b]pyridin-4-yl)amino]cyclohexanecarboxylic acid (Compound No. 77);
4-[(1-Ethyl-5-{5-[2-(ethylamino)-2-oxoethyl]-5-(ethylcarbamoyl)-4,5-dihydroisoxazol-3-yl}-1H-pyrazolo[3,4-b]pyridin-4-yl)amino]cyclohexanecarboxylic acid (Compound No. 78);
4-[(1-Ethyl-5-{5-[2-(methylamino)-2-oxoethyl]-5-(methylcarbamoyl)-4,5-dihydroisoxazol-3-yl}-1H-pyrazolo[3,4-b]pyridin-4-yl)amino]cyclohexanecarboxylic acid (Compound No. 79);
4-[(1-Ethyl-3-methyl-5-{5-[2-(methylamino)-2-oxoethyl]-5-(methylcarbamoyl)-4,5-dihydroisoxazol-3-yl}-1H-pyrazolo[3,4-b]pyridin-4-yl)amino]cyclohexanecarboxylic acid (Compound No. 80);
4-({5-[5-(2-Amino-2-oxoethyl)-5-carbamoyl-4,5-dihydroisoxazol-3-yl]-1-ethyl-3-methyl-1H-pyrazolo[3,4-b]pyridin-4-yl}amino)cyclohexanecarboxylic acid (Compound No. 81);
4-({5-[5-(2-Amino-2-oxoethyl)-5-carbamoyl-4,5-dihydroisoxazol-3-yl]-1-ethyl-1H-pyrazolo[3,4-b]pyridin-4-yl}amino)cyclohexanecarboxylic acid (Compound No. 82);

3-[4-(Cyclohexylamino)-1-ethyl-1H-pyrazolo[3,4-b]pyridin-5-yl]isoxazole-5,5(4H)-dicarboxamide (Compound No. 83);
3-[1-Ethyl-4-(tetrahydro-2H-pyran-4-ylamino)-1H-pyrazolo[3,4-b]pyridin-5-yl]isoxazole-5,5(4H)-dicarboxamide (Compound No. 84);
3-{1-Ethyl-4-[(4-oxocyclohexyl)amino]-1H-pyrazolo[3,4-b]pyridin-5-yl}isoxazole-5,5(4H)-dicarboxamide (Compound No. 85);
3-{1-Ethyl-4-[(4-oxocyclohexyl)amino]-1H-pyrazolo[3,4-b]pyridin-5-yl}-N,N′-dimethylisoxazole-5,5(4H)-dicarboxamide (Compound No. 86);
N,N′-diethyl-3-{1-ethyl-4-[(4-oxocyclohexyl)amino]-1H-pyrazolo[3,4-b]pyridin-5-yl}isoxazole-5,5(4H)-dicarboxamide (Compound No. 87);
3-[4-(Cyclohexylamino)-1-ethyl-1H-pyrazolo[3,4-b]pyridin-5-yl]-N,N′-dimethylisoxazole-5,5(4H)-dicarboxamide (Compound No. 88);
3-[1-Ethyl-4-(tetrahydro-2H-pyran-4-ylamino)-1H-pyrazolo[3,4-b]pyridin-5-yl]-N,N′-dimethylisoxazole-5,5(4H)-dicarboxamide (Compound No. 89);
N,N′-diethyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 90);
3-[4-(Cyclohexylamino)-1-ethyl-1H-pyrazolo[3,4-b]pyridin-5-yl]-N,N′-diethylisoxazole-5,5(4H)-dicarboxamide (Compound No. 91);
N,N′-dicyclobutyl-3-{1-ethyl-4-[(4-oxocyclohexyl)amino]-1H-pyrazolo[3,4-b]pyridin-5-yl}isoxazole-5,5(4H)-dicarboxamide (Compound No. 92);
N,N′-dicyclobutyl-3-[4-(cyclohexylamino)-1-ethyl-1H-pyrazolo[3,4-b]pyridin-5-yl]isoxazole-5,5(4H)-dicarboxamide (Compound No. 93);
N,N′-dicyclobutyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 94);
N,N′-dicyclopentyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 95);
3-[4-(Cyclohexylamino)-1-ethyl-1H-pyrazolo[3,4-b]pyridin-5-yl]-N,N′-dicyclopentylisoxazole-5,5(4H)-dicarboxamide (Compound No. 96);
N,N′-dicyclopentyl-3-{1-ethyl-4-[(4-oxocyclohexyl)amino]-1H-pyrazolo[3,4-b]pyridin-5-yl}isoxazole-5,5(4H)-dicarboxamide (Compound No. 97);
N,N′-dicyclohexyl-3-{1-ethyl-4-[(4-oxocyclohexyl)amino]-1H-pyrazolo[3,4-b]pyridin-5-yl}isoxazole-5,5(4H)-dicarboxamide (Compound No. 98);
N,N′-dicyclohexyl-3-[4-(cyclohexylamino)-1-ethyl-1H-pyrazolo[3,4-b]pyridin-5-yl]isoxazole-5,5(4H)-dicarboxamide (Compound No. 99);
N,N′-dicyclohexyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 100);
3-[1-Ethyl-4-(tetrahydro-2H-pyran-4-ylamino)-1H-pyrazolo[3,4-b]pyridin-5-yl]-N,N′-bis(1-methylcyclohexyl)isoxazole-5,5(4H)-dicarboxamide (Compound No. 101);
3-[4-(Cyclohexylamino)-1-ethyl-1H-pyrazolo[3,4-b]pyridin-5-yl]-N,N′-bis(1-methylcyclohexyl)isoxazole-5,5(4H)-dicarboxamide (Compound No. 102);
3-{1-Ethyl-4-[(4-oxocyclohexyl)amino]-1H-pyrazolo[3,4-b]pyridin-5-yl}-N,N′-bis(1-methylcyclohexyl)isoxazole-5,5(4H)-dicarboxamide (Compound No. 103);
3-{1-Ethyl-4-[(4-oxocyclohexyl)amino]-1H-pyrazolo[3,4-b]pyridin-5-yl}-N,N′-bis(pyridin-4-ylmethyl)isoxazole-5,5(4H)-dicarboxamide (Compound No. 104);
3-[4-(Cyclohexylamino)-1-ethyl-1H-pyrazolo[3,4-b]pyridin-5-yl]-N,N′-bis(pyridin-4-ylmethyl)isoxazole-5,5(4H)-dicarboxamide (Compound No. 105);
3-[1-Ethyl-4-(tetrahydro-2H-pyran-4-ylamino)-1H-pyrazolo[3,4-b]pyridin-5-yl]-N,N′-bis(pyridin-4-ylmethyl)isoxazole-5,5(4H)-dicarboxamide (Compound No. 106);
3-[1-Ethyl-4-(tetrahydro-2H-pyran-4-ylamino)-1H-pyrazolo[3,4-b]pyridin-5-yl]-N,N′-bis(4-fluorophenyl)isoxazole-5,5(4H)-dicarboxamide (Compound No. 107);
3-[4-(Cyclohexylamino)-1-ethyl-1H-pyrazolo[3,4-b]pyridin-5-yl]-N,N′-bis(4-fluorophenyl)isoxazole-5,5(4H)-dicarboxamide (Compound No. 108);
3-{1-Ethyl-4-[(4-oxo cyclohexyl)amino]-1H-pyrazolo[3,4-b]pyridin-5-yl}-N,N′-bis(4-fluorophenyl)isoxazole-5,5(4H)-dicarboxamide (Compound No. 109);
4-({5-[5,5-Bis(pyrrolidin-1-ylcarbonyl)-4,5-dihydroisoxazol-3-yl]-1-ethyl-1H-pyrazolo[3,4-b]pyridin-4-yl}amino)cyclohexanone (Compound No. 110);
5-[5,5-Bis(pyrrolidin-1-ylcarbonyl)-4,5-dihydroisoxazol-3-yl]-N-cyclohexyl-1-ethyl-1H-pyrazolo[3,4-b]pyridin-4-amine (Compound No. 111);
5-[5,5-Bis(pyrrolidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 112);
5-[5,5-Bis(piperidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 113);
5-[5,5-Bis(piperidin-1-ylcarbonyl)-4,5-dihydroisoxazol-3-yl]-N-cyclohexyl-1-ethyl-1H-pyrazolo[3,4-b]pyridin-4-amine (Compound No. 114);
4-({5-[5,5-Bis(piperidin-1-ylcarbonyl)-4,5-dihydroisoxazol-3-yl]-1-ethyl-1H-pyrazolo[3,4-b]pyridin-4-yl}amino)cyclohexanone (Compound No. 115);
4-{[5-(5,5-Bis {[4-(hydroxymethyl)piperidin-1-yl]carbonyl}-4,5-dihydroisoxazol-3-yl)-1-ethyl-1H-pyrazolo[3,4-b]pyridin-4-yl]amino}cyclohexanone (Compound No. 116);
4-[(5-{5,5-Bis[(4-methylpiperazin-1-yl)carbonyl]-4,5-dihydroisoxazol-3-yl}-1-ethyl-1H-pyrazolo[3,4-b]pyridin-4-yl)amino]cyclo hexanone (Compound No. 117);
5-{5,5-Bis[(4-methylpiperazin-1-yl)carbonyl]-4,5-dihydroisoxazol-3-yl}-N-cyclohexyl-1-ethyl-1H-pyrazolo[3,4-b]pyridin-4-amine (Compound No. 118);
5-{5,5-Bis[(4-methylpiperazin-1-yl)carbonyl]-4,5-dihydroisoxazol-3-yl}-1-ethyl-N-(tetrahydro-2H-pyran-4-yl)-1H-pyrazolo[3,4-b]pyridin-4-amine (Compound No. 119);
5-[5,5-Bis(piperazin-1-ylcarbonyl)-4,5-dihydroisoxazol-3-yl]-N-cyclohexyl-1-ethyl-1H-pyrazolo[3,4-b]pyridin-4-amine (Compound No. 120);
4-({5-[5,5-Bis(piperazin-1-ylcarbonyl)-4,5-dihydroisoxazol-3-yl]-1-ethyl-1H-pyrazolo[3,4-b]pyridin-4-yl}amino)cyclohexanone (Compound No. 121);
5-[5,5-Bis(piperazin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 122);
5-{5,5-Bis[(3-benzyl-3,6-diazabicyclo [3.1.0]hex-6-yl)carbonyl]-4,5-dihydroisoxazol-3-yl}-N-cyclohexyl-1-ethyl-1H-pyrazolo[3,4-b]pyridin-4-amine (Compound No. 123);
5-{5,5-Bis[(3-benzyl-3,6-diazabicyclo[3.1.0]hex-6-yl)carbonyl]-4,5-dihydroisoxazol-3-yl}-1-ethyl-N-(tetrahydro-2H-pyran-4-yl)-1H-pyrazolo[3,4-b]pyridin-4-amine (Compound No. 124);
4-[(5-{5,5-Bis[(3-benzyl-3,6-diazabicyclo[3.1.0]hex-6-yl)carbonyl]-4,5-dihydroisoxazol-3-yl}-1-ethyl-1H-pyrazolo[3,4-b]pyridin-4-yl)amino]cyclohexanone (Compound No. 125);
N,N′-bis(3-benzyl-3-azabicyclo[3.1.0]hex-6-yl)-3-{1-ethyl-4-[(4-oxocyclohexyl)amino]-1H-pyrazolo[3,4-b]pyridin-5-yl} isoxazole-5,5(4H)-dicarboxamide (Compound No. 126);
N,N′-bis(3-benzyl-3-azabicyclo[3.1.0]hex-6-yl)-3-[4-(cyclohexylamino)-1-ethyl-1H-pyrazolo[3,4-b]pyridin-5-yl]isoxazole-5,5(4H)-dicarboxamide (Compound No. 127);
N,N′-bis(3-benzyl-3-azabicyclo [3.1.0]hex-6-yl)-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 128);
5-(2-Amino-2-oxoethyl)-3-[1-ethyl-4-(pyridin-3-ylamino)-1H-pyrazolo[3,4-b]pyridin-5-yl]-4,5-dihydroisoxazole-5-carboxamide (Compound No. 129);
5-(2-Amino-2-oxoethyl)-3-[1-ethyl-4-(pyrazin-2-ylamino)-1H-pyrazolo[3,4-b]pyridin-5-yl]-4,5-dihydroisoxazole-5-carboxamide (Compound No. 130);
5-(2-Amino-2-oxoethyl)-3-[1-ethyl-4-(pyrimidin-2-ylamino)-1H-pyrazolo[3,4-b]pyridin-5-yl]-4,5-dihydroisoxazole-5-carboxamide (Compound No. 131);
5-(2-Amino-2-oxoethyl)-3-[1-ethyl-4-(1,2,4-triazin-5-ylamino)-1H-pyrazolo[3,4-b]pyridin-5-yl]-4,5-dihydroisoxazole-5-carboxamide (Compound No. 132);
5-(2-Amino-2-oxoethyl)-3-[1-ethyl-4-(pyrimidin-5-ylamino)-1H-pyrazolo[3,4-b]pyridin-5-yl]-4,5-dihydroisoxazole-5-carboxamide (Compound No. 133);
5-(2-Amino-2-oxoethyl)-3-[1-ethyl-4-(1,3-thiazol-4-ylamino)-1H-pyrazolo[3,4-b]pyridin-5-yl]-4,5-dihydroisoxazole-5-carboxamide (Compound No. 134);
5-(2-Amino-2-oxoethyl)-3-[1-ethyl-4-(4H-1,2,4-triazol-4-ylamino)-1H-pyrazolo[3,4-b]pyridin-5-yl]-4,5-dihydroisoxazole-5-carboxamide (Compound No. 135);
5-(2-Amino-2-oxoethyl)-3-[1-ethyl-4-(pyridin-4-ylamino)-1H-pyrazolo[3,4-b]pyridin-5-yl]-4,5-dihydroisoxazole-5-carboxamide (Compound No. 136);
5-(2-Amino-2-oxoethyl)-3-[1-ethyl-4-(2H-tetrazol-5-ylamino)-1H-pyrazolo[3,4-b]pyridin-5-yl]-4,5-dihydroisoxazole-5-carboxamide (Compound No. 137);
5-(2-Amino-2-oxoethyl)-3-[1-ethyl-4-(1H-tetrazol-5-ylamino)-1H-pyrazolo[3,4-b]pyridin-5-yl]-4,5-dihydroisoxazole-5-carboxamide (Compound No. 138);
5-(2-Amino-2-oxoethyl)-3-[1-ethyl-4-(furan-3-ylamino)-1H-pyrazolo[3,4-b]pyridin-5-yl]-4,5-dihydroisoxazole-5-carboxamide (Compound No. 139);
3-[1-Ethyl-4-(pyridin-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 140);
3-[1-Ethyl-4-(pyrazin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 141);
3-[1-Ethyl-4-(pyrimidin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 142);
3-[1-Ethyl-4-(1,2,4-triazin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 143);
3-[1-Ethyl-4-(pyrimidin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 144);
3-[1-Ethyl-4-(1,3-thiazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 145);
3-[1-Ethyl-4-(4H-1,2,4-triazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 146);
3-[1-Ethyl-4-(pyridin-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 147);
3-[1-Ethyl-4-(2H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 148);
3-[1-Ethyl-4-(1H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 149);
3-[1-Ethyl-4-(furan-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 150);
Methyl 3-{4-[(1,1-dioxidotetrahydro-2H-thiopyran-4-yl)amino]-1-ethyl-1H-pyrazolo[3,4-b]pyridin-5-yl}-5-(2-methoxy-2-oxo ethyl)-4,5-dihydroisoxazole-5-carboxylate (Compound No. 151);
tert-Butyl 3-({5-[5,5-bis(hydroxymethyl)-4,5-dihydroisoxazol-3-yl]-1-ethyl-1H-pyrazolo[3,4-b]pyridin-4-yl}amino)cyclobutanecarboxylate (Compound No. 152);
Methyl 3-[1-ethyl-4-(pyridin-4-ylamino)-1H-pyrazolo[3,4-b]pyridin-5-yl]-5-(2-methoxy-2-oxoethyl)-4,5-dihydroisoxazole-5-carboxylate (Compound No. 153);
3-{4-[(1,1-Dioxidotetrahydro-2H-thiopyran-4-yl)amino]-1-ethyl-1H-pyrazolo[3,4-b]pyridin-5-yl}isoxazole-5,5(4H)-dicarboxylic acid (Compound No. 154);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)diacetic acid (Compound No. 155); and
4-({5-[5,5-Bis(hydroxymethyl)-4,5-dihydroisoxazol-3-yl]-1-ethyl-1H-pyrazolo[3,4-b]pyridin-4-yl}amino)cyclohexanone (Compound No. 156).
   or their pharmaceutically acceptable salts, pharmaceutically acceptable solvates, stereoisomers, tautomers, racemates, regioisomers, geometric isomers, prodrugs, metabolites, polymorphs or N-oxides.
   $ indicates tentatively assigned stereochemistry.

The term “pharmaceutically acceptable” means approved by regulatory agency of the federal or a state government or listed in the U.S. Pharmacopoeia or other generally recognized pharmacopoeia for use in mammals, and more particularly in humans.

The term “pharmaceutically acceptable salts” refers to the derivates of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The term “pharmaceutically acceptable solvates” refers to solvates with water such as hydrates, hemihydrate or sesquihydrate, or pharmaceutically acceptable solvents, for example solvates with common organic solvents as ethanol and the like. Such solvates are also encompassed within the scope of the disclosure.

The present invention also includes within its scope prodrugs of these agents. In general, such prodrugs will be functional derivatives of these compounds, which are readily convertible in vivo into the required compound. Conventional procedures for the selection and preparation of prodrugs are known.

The disclosed compounds may get metabolized in vivo and these metabolites are also encompassed within the scope of this invention.

The term “polymorphs” includes all crystalline form as well as amorphous form for compounds described herein and are included in the present invention.

All stereoisomers of the compounds of the invention are contemplated, either in admixture or in pure form. The compounds of the present invention can have asymmetric centers at any of the carbon atoms including all the substituents. Consequently, compounds of present invention can exist in enantiomeric or diastereomeric forms or in mixture thereof. The processes for the preparation can utilize racemates, enantiomers, or diastereomers as starting materials. When diastereomeric or enantiomeric products are prepared, they can be separated by conventional methods, for example, chromatographic or fractional crystallization.

The term “tautomer” includes one of two or more structural isomers that exist in equilibrium and are readily converted from one isomeric form to another. Certain compounds of the invention may furthermore be present in tautomeric forms.

The term “regioisomers” refers to compounds, which have the same molecular formula but differ in the connectivity of the atoms.

The term, “geometric isomers”, refers to compounds, having the same molecular formula as another but a different geometric configuration, as when atoms or groups of atoms are attached in different spatial arrangements on either side of a double bond or other rigid bond.

The term “racemate” includes a mixture of equal amounts of left- and right-handed stereoisomers of chiral molecules.

When a bond to a substituent is shown to cross a bond connecting two atoms in a ring, then such substituent may be bonded to any atom on the ring.

In another aspect, the present invention includes pharmaceutical compositions comprising, as an active ingredient, at least one of the disclosed compound or a pharmaceutically acceptable salt, a pharmaceutically acceptable solvate, stereoisomer, tautomer, racemate, regioisomer, geometric isomer, prodrug, metabolite, polymorph or N-oxide, along with a pharmaceutically acceptable carrier, excipient or diluent. Compounds disclosed herein may be administered to mammal for treatment by any route, which effectively transports the active compound to the appropriate or desired site of action such as oral, nasal, pulmonary, transdermal or parenteral (rectal, subcutaneous, intravenous, intraurethral, intramuscular, intranasal). The pharmaceutical composition of the present invention comprises a pharmaceutically effective amount of a compound of the present invention formulated along with one or more pharmaceutically acceptable carriers, excipients or diluents. The choice of pharmaceutical carrier, excipient or diluent can be made with regard to the intended route of administration and standard pharmaceutical practice.

Where desired, the compounds of the invention and/or their pharmaceutically acceptable salts, pharmaceutically acceptable solvates, stereoisomers, tautomers, racemates, regioisomers, geometric isomers, prodrugs, metabolites, polymorphs or N-oxides may be advantageously used in combination with one or more other compounds. Examples of other compounds, which may be used in combination with compounds of this invention and/or their pharmaceutically acceptable salts, pharmaceutically acceptable solvates, stereoisomers, tautomers, racemates, regioisomers, geometric isomers, prodrugs, metabolites, polymorphs or N-oxides include β2-agonists, corticosteroids, leukotriene antagonists, 5-lipoxygenase inhibitors, chemokine inhibitors, p38 kinase inhibitors, anticholinergics, antiallergics, PAF (platelet activating factor) antagonists, EGFR (epidermal growth factor receptor) kinase inhibitors, muscarinic receptor antagonists or combination(s) thereof.

The one or more β2-agonist as described herein may be chosen from those described in the art. The β2-agonists may include one or more compounds described in U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258.

β2-agonists include, for example, one or more of albuterol, salbutamol, biltolterol, pirbuterol, levosalbutamol, tulobuterol, terbutaline, bambuterol, metaproterenol, fenoterol, salmeterol, carmoterol, arformoterol, formoterol, and their pharmaceutically acceptable salts or solvates thereof.

Corticosteroids as described herein may be chosen from those described in the art. Corticosteroids may include one or more compounds described in U.S. Pat. Nos. 3,312,590; 3,983,233; 3,929,768; 3,721,687; 3,436,389; 3,506,694; 3,639,434; 3,992,534; 3,928,326; 3,980,778; 3,780,177; 3,652,554; 3,947,478; 4,076,708; 4,124,707; 4,158,055; 4,298,604; 4,335,121; 4,081,541; 4,226,862; 4,290,962; 4,587,236; 4,472,392; 4,472,393; 4,242,334; 4,014,909; 4,098,803; 4,619,921; 5,482,934; 5,837,699; 5,889,015; 5,278,156; 5,015,746; 5,976,573; 6,337,324; 6,057,307; 6,723,713; 6,127,353; and 6,180,781.

Corticosteroids may include, for example, one or more of alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, hydrocortisone, methylprednisolone, mometasone, prednicarbate, prednisolone, rimexolone, tixocortol, triamcinolone, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone. Examples of possible salts or derivatives include: sodium salts, sulfobenzoates, phosphates, isonicotinates, acetates, propionates, dihydrogen phosphates, palmitates, pivalates, or furoates. In some cases, the corticosteroids may also occur in the form of their hydrates.

The leukotriene antagonist can be selected from compounds, for example, those described in U.S. Pat. No. 5,565,473; 5,583,152; 4,859,692 or 4,780,469.

Examples of leukotriene antagonist include, but are not limited to, montelukast, zafirlukast, pranlukast and pharmaceutically acceptable salts thereof

5-Lipoxygenase inhibitors can be selected from for example, compounds in U.S. Pat. Nos. 4,826,868, or 4,873,259, or European Patent Nos. 0 419 049, 0 542 356 or 0 542 355. Examples may include, but are not limited to, atreleuton, zyflo (zileuton), ABT-761, fenleuton or tepoxalin.

Examples of the chemokine inhibitors include, but are not limited to, endogenous ligands of chemokine receptors or derivatives thereof, and non-peptidic low molecular compounds or antibodies for chemokine receptors.

Examples of the endogenous ligands of chemokine receptors include, but are not limited to, MIP-1α, MIP-1β, Rantes, SDF-1α, SDF-1β, MCP-1, MCP-2, MCP4, Eotaxin, and MDC. Examples of the derivatives of endogenous ligands include, but are not limited to, AOP-RANTES, Met-SDF-1α, and Met-SDF-1β.

Examples of the antibodies for chemokine receptors include, but are not limited to, Pro-140.

Examples of the non-peptidic low molecular compounds include, but are not limited to, antagonists and agonists for CCR1, CCR2, CCR3, CCR4, CCR5, CXCR1, CXCR2, CXCR3 and CXCR4 receptors.

p38 kinase inhibitors include compounds disclosed in WO 2006/021848, WO 2006/016237, WO 2006/056863, WO 2006/117657 and WO 2006/082492. Any reference to the above mentioned p38 kinase inhibitors also includes any pharmacologically acceptable salts thereof which may exist.

Anticholinergics include, for example, tiotropium salts, ipratropium salts, oxitropium salts, salts of the compounds known from WO 02/32899: tropenol N-methyl-2,2-diphenylpropionate, scopine N-methyl-2,2-diphenylpropionate, scopine N-methyl-2-fluoro-2,2-diphenylacetate and tropenol N-methyl-2-fluoro-2,2-diphenylacetate; as well as salts of the compounds known from WO 02/32898: tropenol N-methyl-3,3′,4,4′-tetrafluorobenzilate, scopine N-methyl-3,3′,4,4′-tetrafluorobenzilate, scopine N-methyl-4,4′-dichlorobenzilate, scopine N-methyl-4,4′-difluorobenzilate, tropenol N-methyl-3,3′-difluorobenzilate, scopine N-methyl-3,3′-difluorobenzilate, and tropenol N-ethyl-4,4′-difluorobenzilate, optionally in the form of their hydrates and solvates. By salts are meant those compounds which contain, in addition to the above mentioned cations, as counter-ion, an anion with a single negative charge selected from among the chloride, bromide, and methanesulfonate.

Preferred anticholinergics include, for example, tiotropium bromide, ipratropium bromide, oxitropium bromide, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide, scopine 4,4′-dichlorobenzilate methobromide, scopine 4,4′-difluorobenzilate methobromide, tropenol 3,3′-difluorobenzilate methobromide, scopine 3,3′-difluorobenzilate methobromide, and tropenol 4,4′-difluorobenzilate ethylbromide.

Antiallergics include, for example, epinastine, cetirizine, azelastine, fexofenadine, levocabastine, loratadine, mizolastine, ketotifene, emedastine, dimetindene, clemastine, bamipine, hexachloropheniramine, pheniramine, doxylamine, chlorophenoxamine, dimenhydrinate, diphenhydramine, promethazine, ebastine, desloratadine, and meclizine. Preferred antiallergic agents include, for example, epinastine, cetirizine, azelastine, fexofenadine, levocabastine, loratadine, ebastine, desloratadine, and mizolastine. Any reference to the above-mentioned antiallergic agents also includes any pharmacologically acceptable salts thereof, which may exist.

PAF antagonists include, for example, 4-(2-chlorophenyl)-9-methyl-2-[3-(4-morpholinyl)-3-propanon-1-yl]-6H-thieno[3,2-f][1,2,4]triazolo[4,3-a][1,4]diazepine and 6-(2-chlorophenyl)-8,9-dihydro-1-methyl-8-[(4-morpholinyl)carbonyl]-4H,7H-cyclopenta[4.5]thieno[3,2-f][1,2,4]triazolo[4,3-a][1,4]diazepine.

EGFR kinase inhibitors include, for example, 4-[(3-chloro-4-fluorophenyl)amino]-7-(2-{4-[(S)-(2-oxotetrahydrofuran-5-yl)carbonyl]piperazin-1-yl}-ethoxy)-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]-amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropyl-methoxyquinazoline, and 4-[(3-chloro-4-fluorophenyl)amino]-6-[3-(morpholin-4-yl)propyloxy]-7-methoxyquinazoline. Any reference to the above-mentioned EGFR kinase inhibitors also includes any pharmacologically acceptable salts thereof which may exist.

Muscarinic receptor antagonists include substances that directly or indirectly block activation of muscarinic cholinergic receptors. Examples include, but are not limited to, quaternary amines (e.g., methantheline, ipratropium, propantheline), tertiary amines (e.g., dicyclomine, scopolamine) and tricyclic amines (e.g., telenzepine). Other muscarinic receptor antagonists include benztropine, hexahydro-sila-difenidol hydrochloride (HHSID hydrochloride), (+/−)-3-quinuclidinyl xanthene-9-carboxylate hemioxalate (QNX-hemioxalate), telenzepine dihydrochloride, tolterodine, oxybutynin and atropine.

Examples set forth below demonstrate the synthetic procedures for the preparation of the representative compounds. The examples are provided to illustrate particular aspect of the disclosure and do not constrain the scope of the present invention as defined by the claims.

### EXPERIMENTAL DETAILS

### Example 1a

### Preparation of 1-(4-methoxybenzyl)-1H-pyrazol-5-amine

This compound was synthesized according to procedure reported in Bio. and Med. Chem. Lett., 13 (2003), 1133-1136.

### Example 1b

### Preparation of 1-ethyl-3-methyl-1H-pyrazol-5-amine

This compound was synthesized according to procedure reported in Chem. Pharm. Bull. 52 (9): (2004), 1098-1104.

### Example 1c

### Preparation of tetrahydro-2H-pyran-4-amine hydrochloride

This compound was synthesized according to the procedure reported in Tetrahedron Letters, 42, (2001), 4257-4259.

### Example 1d

### Preparation of tetrahydro-2H-thiopyran-4-amine

Step a: Tetrahydro-4H-thiopyran-4-one (15 gm, 0.129 mole), hydroxylamine hydrochloride (15.27 gm, 0.219 mole) and sodium acetate trihydrate (30 gm, 0.219 mole) were taken together in a mixture of water (150 ml) and ethanol (60 ml). The reaction mixture was refluxed for about 4 hours. The solvent was evaporated under reduced pressure. Solid compound, which separated out, was filtered and dried under vacuum.

Yield: 15 gm (99%)

Step b: Lithium aluminum hydride (6.96 gm, 0.183 mole) was taken in tetrahydrofuran (80 ml) and solution of tetrahydro-4H-thiopyran-4-one oxime (8 gm, 0.0610 mole) (step a) in tetrahydrofuran (20 ml) was added to it drop wise at 0° C. The reaction mixture was refluxed for about 4 hours and quenched with saturated ammonium chloride solution. Extraction was done using ethyl acetate, organic layer was dried over anhydrous sodium sulfate and concentrated under reduced pressure to get the title compound.

Yield: 8 gm (crude) (100%)

### Example 2

### Preparation of diethyl {[(1-ethyl-1H-pyrazol-5-yl)amino]methylidene}propanedioate

A mixture of 5-amino-1-ethylpyrazole (5 gm, 0.0448 mole) and diethylethoxy methylenemalonate (10.35 ml, 0.0448 mole) was stirred at 120° C. for about 1 hour. The reaction mixture was poured into water and extraction was done with ethyl acetate. The organic layer was dried over anhydrous sodium sulfate and concentrated in vacuo to give viscous oil.

Yield: 15 gm (crude) (124%)

The following compounds were prepared similarly:
Diethyl {[(1,3-dimethyl-1H-pyrazol-5-yl)amino]methylidene}propanedioate
Diethyl ({[1-(4-methoxybenzyl)-1H-pyrazol-5-yl]amino}methylidene)propanedioate

The following compound can be prepared similarly:
Diethyl {[(1-ethyl-3-methyl-1H-pyrazol-5-yl)amino]methylidene}propanedioate

### Example: 2a

### Preparation of ethyl 4-hydroxy-1-(4-methoxybenzyl)-1H-pyrazolo[3,4-b]pyridine-5-carboxylate

Diphenyl ether (180 ml) was heated to about 230° C. (Internal temperature 200-210° C.) under inert atmosphere in a round bottom flask fitted with distillation set and a solution of diethyl ({[1-(4-methoxybenzyl)-1H-pyrazol-5-yl]amino}methylidene)propanedioate (85 gm, 0.227 mol) (example 2) in absolute ethanol (130 ml) was added dropwise. The reaction mixture was heated for about 2 hours. Volatile solubles were distilled out. It was cooled to 45° C. and methanol (150 ml) was added dropwise. Solid, which precipitated out was filtered and washed with methanol and hexane and dried under vacuum.

Yield: 33 gm (crude) (45%)

m/z: (M 41) 328.10

### Example 3

### Preparation of ethyl 4-chloro-1-ethyl-1H-pyrazolo [3,4-b]pyridine-5-carboxylate

A mixture of diethyl {[(1-ethyl-1H-pyrazol-5-yl)amino]methylidene}propanedioate (15 gm, 0.0533 mole) (example 2) and phosphorous oxy chloride (76.64 ml, 0.7998 mole) was heated at 110-120° C. under stirring for about 4 hours under argon atmosphere. The reaction mixture was cooled and then poured drop wise into ice water. A pale yellow solid separated which was filtered. The solid was first washed twice with ice cold water and then finally with hexane and dried over vacuum.

Yield: 10 gm (70%)

m/z: (M⁺+1) 254.2

The following compound was prepared similarly:
Ethyl 4-chloro-1,3-dimethyl-1H-pyrazolo[3,4-b]pyridine-5-carboxylate

The following compound can be prepared similarly:
Ethyl 4-chloro-1-ethyl-3-methyl-1H-pyrazolo[3,4-b]pyridine-5-carboxylate

### Example 3a

### Preparation of ethyl 4-chloro-1-(4-methoxybenzyl)-1H-pyrazolo[3,4-b]pyridine-5-carboxylate

The title compound was prepared by following the procedure of example 3 using ethyl 4-hydroxy-1-(4-methoxybenzyl)-1H-pyrazolo[3,4-b]pyridine-5-carboxylate (example 2a).

m/z: (M 41) 346.09

### Example 4

### Preparation of ethyl 4-cyclohexylamino-1-ethyl-1H-pyrazolo[3,4-b]pyridine-5-carboxylate

Cyclohexyl amine (9.07 ml, 0.7905 mole) was added to a mixture of ethyl 4-chloro-1-ethyl-1H-pyrazolo[3,4-b]pyridine-5-carboxylate (10 gm, 0.0395 mole) (example 3) in acetonitrile. After stirring for about 2 hours at 110° C., acetonitrile was removed under reduced pressure. Water was added and the reaction mixture was extracted with ethyl acetate. The organic layer was washed with brine, dried over anhydrous sodium sulfate and concentrated in vacuo to give brownish solid compound.

Yield: 9.6 gm (78%)

m/z: (M41) 317.22

The following compounds were prepared similarly:
Ethyl 1-ethyl-4-(tetrahydro-2H-pyran-4-ylamino)-1H-pyrazolo[3,4-b]pyridine-5-carboxylate

m/z: (M41) 319.26
Ethyl 1-ethyl-4-[(4-hydroxycyclohexyl)amino]-1H-pyrazolo[3,4-b]pyridine-5-carboxylate

m/z: (M41) 333.06
Ethyl 4-(cyclopropylamino)-1-ethyl-1H-pyrazolo[3,4-b]pyridine-5-carboxylate

m/z: (M41) 275.0
Ethyl 4-(cyclopropylamino)-1,3-dimethyl-1H-pyrazolo[3,4-b]pyridine-5-carboxylate
Ethyl 4-(cyclopentylamino)-1-ethyl-1H-pyrazolo[3,4-b]pyridine-5-carboxylate
Ethyl 4-(cyclopentylamino)-1,3-dimethyl-1H-pyrazolo[3,4-b]pyridine-5-carboxylate
Ethyl 1-(4-methoxybenzyl)-4-(tetrahydro-2H-thiopyran-4-ylamino)-1H-pyrazolo[3,4-b]pyridine-5-carboxylate

m/z: (M41) 427.14
Ethyl 4-(cyclohexylamino)-1-(4-methoxybenzyl)-1H-pyrazolo[3,4-b]pyridine-5-carboxylate

m/z: (M⁺1 409.22
Ethyl 4-(benzylamino)-1-(4-methoxybenzyl)-1H-pyrazolo[3,4-b]pyridine-5-carboxylate

m/z: (M 41) 417.14
Ethyl 1-ethyl-4-(tetrahydro-2H-thiopyran-4-ylamino)-1H-pyrazolo[3,4-b]pyridine-5-carboxylate

### Example 4a

### Preparation of 4-chloro-1-ethyl-1H-pyrazolo[3,4-b]pyridine-5-carboxylic acid

A solution of ethyl 4-chloro-1-ethyl-1H-pyrazolo[3,4-b]pyridine-5-carboxylate (0.013 mol) (example 3) in dioxane is treated with potassium hydroxide (0.13 mol in 30 ml water) solution. The reaction mixture is stirred for about 3-4 hours and concentrated under reduced pressure. It is acidified with hydrochloric acid to pH of about 3-4, extracted with ethyl acetate, washed with brine and dried under vacuo.

### Example 4b

### Preparation of 4-{[4-(tert-butoxycarbonyl)cyclohexyl]amino}-1-ethyl-1H-pyrazolo[3,4-b]pyridine-5-carboxylic acid

A solution of 4-chloro-1-ethyl-1H-pyrazolo[3,4-b]pyridine-5-carboxylic acid (0.0088 mol) (example 4a) in acetonitrile is treated with tert-butyl 4-aminocyclohexanecarboxylate (0.026 mol). The reaction mixture is refluxed for about 3-4 hours. Solvent is evaporated off and water is added and extraction is done with ethyl acetate. The organic layer is washed with brine, dried and concentrated under reduced pressure to give crude compound, which is purified by column chromatography.

The following compound can be prepared similarly:
4-{[3-(tert-butoxycarbonyl)cyclobutyl]amino}-1-ethyl-1H-pyrazolo[3,4-b]pyridine-5-carboxylic acid

### Example 4c

### Preparation of ethyl 4-[(1,1-dioxidotetrahydro-2H-thiopyran-4-yl)amino]-1-(4-methoxybenzyl)-1H-pyrazolo[3,4-b]pyridine-5-carboxylate

Ethyl 1-(4-methoxybenzyl)-4-(tetrahydro-2H-thiopyran-4-ylamino)-1H-pyrazolo[3,4-b]pyridine-5-carboxylate (500 mg, 0.00117 mole) (example 4) was taken in dichloromethane (5 ml). At 0° C., m-chloroperbenzoic acid (600 mg, 0.00352 mole) was added and the mixture was stirred overnight. Water was added and extraction was done using dichloromethane. The organic layer was washed with saturated ammonium bicarbonate and then with brine, dried over anhydrous sodium sulfate and concentrated under reduced pressure to get the title compound.

Yield: 500 mg (93%)

m/z: (M⁺+1) 495.16

The following compound can be prepared similarly:
Ethyl 4-[(1,1-dioxidotetrahydro-2H-thiopyran-4-yl)amino]-1-ethyl-1H-pyrazolo[3,4-b]pyridine-5-carboxylate

### Example 5

### Preparation of 4-cyclohexylamino-1-ethyl-1H-pyrazolo[3,4-b]pyridine-5-carboxylic acid

Sodium hydroxide solution (4.09 gm in 20 ml water) was added to a solution of ethyl 4-cyclohexylamino-1-ethyl-1H-pyrazolo[3,4-b]pyridine-5-carboxylate (9.32 gm, 0.0294 mole) (example 4) in ethanol. The reaction mixture was stirred for about 14 hours at room temperature and then warmed for 1 hour at 60° C. Water was added and the reaction mixture was extracted with ethyl acetate. Aqueous layer was acidified by using hydrochloric acid (2N) to pH of about 4-5. White solid, which was obtained, was filtered and dried in vacuo.

Yield: 9 gm (crude) (100%)

m/z: (M 41) 289.22

The following compounds were prepared similarly:
1-Ethyl-4-(tetrahydro-2H-pyran-4-ylamino)-1H-pyrazolo[3,4-b]pyridine-5-carboxylic acid

m/z: (M41) 291.36
1-Ethyl-4-[(4-hydroxycyclohexyl)amino]-1H-pyrazolo[3,4-b]pyridine-5-carboxylic acid

m/z: (M41) 305.10
4-(Cyclopropylamino)-1-ethyl-1H-pyrazolo[3,4-b]pyridine-5-carboxylic acid

m/z: (M 41) 274.2
4-(Cyclopropylamino)-1,3-dimethyl-1H-pyrazolo[3,4-b]pyridine-5-carboxylic acid
4-(Cyclopentylamino)-1-ethyl-1H-pyrazolo[3,4-b]pyridine-5-carboxylic acid
4-(Cyclopentylamino)-1,3-dimethyl-1H-pyrazolo[3,4-b]pyridine-5-carboxylic acid
4-[(1,1-Dioxidotetrahydro-2H-thiopyran-4-yl)amino]-1-ethyl-1H-pyrazolo[3,4-b]pyridine-5-carboxylic acid
4-[(1,1-Dioxidotetrahydro-2H-thiopyran-4-yl)amino]-1-(4-methoxybenzyl)-1H-pyrazolo[3,4-b]pyridine-5-carboxylic acid

m/z: (M 41) 383.28
4-(Benzylamino)-1-(4-methoxybenzyl)-1H-pyrazolo[3,4-b]pyridine-5-carboxylic acid

m/z: (M 41) 389.08
4-(Cyclohexylamino)-1-(4-methoxybenzyl)-1H-pyrazolo[3,4-b]pyridine-5-carboxylic acid

### Example 6

### Preparation of 4-(cyclohexylamino)-1-ethyl-N-methoxy-N-methyl-1H-pyrazolo[3,4-b]pyridine-5-carboxamide

4-Cyclohexylamino-1-ethyl-1H-pyrazolo[3,4-b]pyridine-5-carboxylic acid (0.200 gm, 0.0006 mole) (example 5) and N,O-dimethylhydroxylamine hydrochloride (0.102 gm, 0.0010 mole) were taken in dimethylformamide. At 0° C., hydroxybenzotriazole (0.162 gm, 0.0012 mole) and N-methylmorpholine (0.30 ml, 0.0027 mole) were added and the reaction mixture was stirred for about 1 hour. 1-Ethyl-3-(3-dimethylaminopropyl) carbodiimide hydrochloride (0.266 gm, 0.0012 mole) was added and the reaction mixture was stirred for about 14 hours. Water was added and extraction was carried out with ethyl acetate. The organic layer was washed with brine, dried over anhydrous sodium sulfate and concentrated in vacuo. The compound was purified by using preparative thin layer chromatography.

Yield: 136 mg (59%)

m/z: (M41) 332.26

The following compounds were prepared similarly:
1-Ethyl-N-methoxy-N-methyl-4-(tetrahydro-2H-pyran-4-ylamino)-1H-pyrazolo[3,4-b]pyridine-5-carboxamide

m/z: (M41) 334.11
1-Ethyl-4-[(4-hydroxycyclohexyl)amino]-N-methoxy-N-methyl-1H-pyrazolo[3,4-b]pyridine-5-carboxamide

m/z: (M41) 348.05
4-(Cyclopropylamino)-1-ethyl-N-methoxy-N-methyl-1H-pyrazolo[3,4-b]pyridine-5-carboxamide

m/z: (M⁺+1) 290.2
4-(Cyclopropylamino)-N-methoxy-N-1,3-trimethyl-1H-pyrazolo[3,4-b]pyridine-5-carboxamide
4-(Cyclopentylamino)-1-ethyl-N-methoxy-N-methyl-1H-pyrazolo[3,4-b]pyridine-5-carboxamide
4-(Cyclopentylamino)-N-methoxy-N-1,3-trimethyl-1H-pyrazolo[3,4-b]pyridine-5-carboxamide
4-[(1,1-Dioxidotetrahydro-2H-thiopyran-4-yl)amino]-1-ethyl-N-methoxy-N-methyl-1H-pyrazolo[3,4-b]pyridine-5-carboxamide

m/z: (M 41) 382.10
4-(B enzylamino)-N-methoxy-1-(4-methoxybenzyl)-N-methyl-1H-pyrazolo[3,4-b]pyridine-5-carboxamide

m/z: (M⁺+1) 432.10
4-[(1,1-Dioxidotetrahydro-2H-thiopyran-4-yl)amino]-N-methoxy-1-(4-methoxybenzyl)-N-methyl-1H-pyrazolo[3,4-b]pyridine-5-carboxamide

m/z: (M⁺+1) 474.06
4-(Cyclohexylamino)-N-methoxy-1-(4-methoxybenzyl)-N-methyl-1H-pyrazolo[3,4-b]pyridine-5-carboxamide

m/z: (M⁺+1) 332.26

The following compounds can be prepared similarly:
tert-butyl 3-({1-ethyl-5-[methoxy(methyl)carbamoyl]-1H-pyrazolo[3,4-b]pyridin-4-yl}amino)cyclobutanecarboxylate
tert-butyl 4-({1-ethyl-5-[methoxy(methyl)carbamoyl]-1H-pyrazolo[3,4-b]pyridin-4-yl}amino)cyclohexanecarboxylate

### Example 6a

### Preparation of 4-(benzylamino)-N-methoxy-N-methyl-1H-pyrazolo[3,4-b]pyridine-5-carboxamide

Trifluoroacetic acid (5.35 ml, 69.6 mmol) was added to the solution of 4-(benzylamino)-N-methoxy-1-(4-methoxybenzyl)-N-methyl-1H-pyrazolo[3,4-b]pyridine-5-carboxamide (3 gm, 6.96 mmol) (example 6) in dichloroethane (20 ml) and the reaction mixture was refluxed for about 2 hours under inert atmosphere. It was cooled, diluted with ethyl acetate, washed with saturated sodium bicarbonate, water and brine, dried over anhydrous sodium sulfate and concentrated under reduced pressure to get the title compound.

Yield: 2 gm (92%)

The following compound was prepared similarly:
4-(Cyclohexylamino)-N-methoxy-N-methyl-1H-pyrazolo[3,4-b]pyridine-5-carboxamide

m/z: (M 41) 304.12

### Example 6b

### Preparation of 4-(benzylamino)-1-ethyl-N-methoxy-N-methyl-1H-pyrazolo[3,4-b]pyridine-5-carboxamide

Ethyl iodide (1.52 gm, 9.63 mmol) and potassium carbonate (2.214 gm, 16.05 mmol) were added to the solution 4-(benzylamino)-N-methoxy-N-methyl-1H-pyrazolo[3,4-b]pyridine-5-carboxamide (1 gm, 3.21 mmol) (example 6a) in dimethylformamide and the reaction mixture was stirred at 60° C. for about 5 hours. It was cooled, diluted with water and extracted with ethyl acetate. The organic layer was washed with brine, dried over anhydrous sodium sulfate and concentrated under reduced pressure. The crude product was purified over silica gel column.

Yield: 0.800 gm (73%)

m/z: (M 41) 340.22

### Example 7

### Preparation of 4-cyclohexylamino-1-ethyl-1H-pyrazolo[3,4-b]pyridine-5-carbaldehyde

Toluene was cooled to −30 to −35° C. and vitride (0.12 ml, 0.0006 mole) was added. After about 10 minutes, 4-(cyclohexylamino)-1-ethyl-N-methoxy-N-methyl-1H-pyrazolo[3,4-b]pyridine-5-carboxamide (0.100 gm, 0.0003 mole) (example 6) was added and the reaction mixture was stirred for about 4 hours. Citric acid solution in water (10%) was added dropwise to quench the reaction and the mixture was extracted with ethyl acetate. The organic layer was washed with brine and dried over anhydrous sodium sulfate and concentrated in vacuo. The compound was purified by using preparative thin layer chromatography.

Yield: 54 mg (65%)

m/z: (M 41) 273.23

The following compounds were prepared similarly.
1-Ethyl-4-(tetrahydro-2H-pyran-4-ylamino)-1H-pyrazolo[3,4-b]pyridine-5-carbaldehyde

m/z: (M41) 275.06
1-Ethyl-4-[(4-hydroxycyclohexyl)amino]-1H-pyrazolo[3,4-b]pyridine-5-carbaldehyde

m/z: (M41) 289.06
4-(Cyclopropylamino)-1-ethyl-1H-pyrazolo[3,4-b]pyridine-5-carbaldehyde

m/z: (M41) 231.1
4-(Cyclopropylamino)-1,3-dimethyl-1H-pyrazolo[3,4-b]pyridine-5-carbaldehyde
4-(Cyclopentylamino)-1-ethyl-1H-pyrazolo[3,4-b]pyridine-5-carb aldehyde
4-(Cyclopentylamino)-1,3-dimethyl-1H-pyrazolo[3,4-b]pyridine-5-carbaldehyde
4-[(1,1-Dioxidotetrahydro-2H-thiopyran-4-yl)amino]-1-ethyl-1H-pyrazolo[3,4-b]pyridine-5-carbaldehyde

m/z: (M41) 323.19
4-(B enzylamino)-1-ethyl-1H-pyrazolo[3,4-b]pyridine-5-carb aldehyde

m/z: (M41) 281.11
4-(Cyclohexylamino)-1-(4-methoxybenzyl)-1H-pyrazolo[3,4-b]pyridine-5-carbaldehyde

m/z: (M41) 365.31

The following compounds can be prepared similarly:
tert-butyl 4-[(1-ethyl-5-formyl-1H-pyrazolo[3,4-b]pyridin-4-yl)amino]cyclohexane carboxylate
tert-butyl 3-[(1-ethyl-5-formyl-1H-pyrazolo[3,4-b]pyridin-4-yl)amino]cyclobutane carboxylate

### Example 8

### Preparation of 4-cyclohexylamino-1-ethyl-1H-pyrazolo[3,4-b]pyridine-5-carbaldehyde oxime

Hydroxylamine hydrochloride (0.255 gm, 0.0036 mole) and sodium acetate (0.301 gm, 0.0036 mole) were added to a stirred solution of 4-cyclohexylamino-1-ethyl-1H-pyrazolo[3,4-b]pyridine-5-carbaldehyde (0.250 gm, 0.0009 mole) (example 7) in ethanol. The reaction mixture was allowed to stir at room temperature for about 2 hours. Ethanol was removed under reduced pressure and the residue was poured in water. The title compound obtained was filtered and washed twice with water and then finally with hexane.

Yield: 202 mg (77%)

m/z: (M 41) 288.31

The following compounds were prepared similarly:
1-Ethyl-4-(tetrahydro-2H-pyran-4-ylamino)-1H-pyrazolo[3,4-b]pyridine-5-carbaldehyde oxime

m/z: (M41) 290.13
1-Ethyl-4-[(4-hydroxycyclohexyl)amino]-1H-pyrazolo[3,4-b]pyridine-5-carbaldehyde oxime

m/z: (M41) 304.11
4-(Cyclopropylamino)-1-ethyl-1H-pyrazolo[3,4-b]pyridine-5-carbaldehyde oxime

m/z: (M41) 246.1
4-(Cyclopropylamino)-1,3-dimethyl-1H-pyrazolo[3,4-b]pyridine-5-carbaldehyde oxime
4-(Cyclopentylamino)-1-ethyl-1H-pyrazolo[3,4-b]pyridine-5-carbaldehyde oxime

4-(Cyclopentylamino)-1,3-dimethyl-1H-pyrazolo[3,4-b]pyridine-5-carbaldehyde oxime
4-[(1,1-Dioxidotetrahydro-2H-thiopyran-4-yl)amino]-1-ethyl-1H-pyrazolo[3,4-b]pyridine-5-carbaldehyde oxime

m/z: (M41) 338.22
4-(B enzylamino)-1-ethyl-1H-pyrazolo[3,4-b]pyridine-5-carb aldehyde oxime
4-(Cyclohexylamino)-1-(4-methoxybenzyl)-1H-pyrazolo[3,4-b]pyridine-5-carbaldehyde oxime

The following compounds can be prepared similarly:
tert-butyl 3-({1-ethyl-5-[(Z)-(hydroxyimino)methyl]-1H-pyrazolo[3,4-b]pyridin-4-yl}amino)cyclobutanecarboxylate
tert-butyl 4-({1-ethyl-5-[(E)-(hydroxyimino)methyl]-1H-pyrazolo[3,4-b]pyridin-4-yl}amino)cyclohexanecarboxylate

### Example 9

### Preparation of {3-[4-(cyclohexylamino)-1-ethyl-1H-pyrazolo[3,4-b]pyridin-5-yl]-4,5-dihydroisoxazole-5,5-diyl}dimethanol (Compound No. 1)

Methylene-1,3-propane-diol (0.07 ml, 0.00084 mol) was added to the solution of 4-cyclohexylamino-1-ethyl-1H-pyrazolo[3,4-b]pyridine-5-carbaldehyde oxime (121 mg, 0.00042 mol) (example 8) in tetrahydrofuran (5 ml), and the resulting reaction mixture was stirred at room temperature. Sodium hypochlorite (2 ml) was added slowly to the mixture thus obtained over the period of about 5 minutes and the reaction mixture was allowed to stir at room temperature for about 14 hours. Tetrahydrofuran was evaporated off and the organic compound was extracted with ethyl acetate twice. The organic layer was concentrated and purified by column chromatography to yield the title compound.

Yield: 41 mg (26%)

m/z: (M41) 374.39

NMR: (δ, CDCl₃): 1.26-2.13 (m, 13H), 3.49 (s, 2H), 3.75 (s, 4H), 4.11-4.13 (m, 1H), 4.44-4.49 (q, 2H) 7.99 (s, 1H), 8.12 (s, 1H).

The following compounds were prepared similarly:
Methyl 3-[4-(cyclohexylamino)-1-ethyl-1H-pyrazolo[3,4-b]pyridin-5-yl]-5-(2-methoxy-2-oxo ethyl)-4,5-dihydroisoxazole-5-carboxylate (Compound No. 2);

Yield: 54%

m/z: (M⁺+1) 444.45
Methyl 3-[1-ethyl-4-(tetrahydro-2H-pyran-4-ylamino)-1H-pyrazolo[3,4-b]pyridin-5-yl]-5-(2-methoxy-2-oxoethyl)-4,5-dihydroisoxazole-5-carboxylate (Compound No. 3);

Yield: 18%

m/z: (M 41) 446.04
{3-[1-Ethyl-4-(tetrahydro-2H-pyran-4-ylamino)-1H-pyrazolo[3,4-b]pyridin-5-yl]-4,5-dihydroisoxazole-5,5-diyl}dimethanol (Compound No. 4);

Yield: 20%

m/z (M41) 376.03
4-({5-[5,5-Bis(hydroxymethyl)-4,5-dihydroisoxazol-3-yl]-1-ethyl-1H-pyrazolo[3,4-b]pyridin-4-yl}amino)cyclohexanol (Compound No. 5);

Yield: 24%

m/z: (M 41) 390.07
Methyl 3-[4-(cyclopropylamino)-1-ethyl-1H-pyrazolo[3,4-b]pyridin-5-yl]-5-methyl-4,5-dihydroisoxazole-5-carboxylate (Compound No. 10);

Yield: 51%

m/z: (M 41) 344.1
3-[4-(Cyclopropylamino)-1-ethyl-1H-pyrazolo[3,4-b]pyridin-5-yl]-5-methyl-4,5-dihydroisoxazole-5-carbonitrile (Compound No. 11);

Yield: 26%

m/z: (M⁺+1) 311.2.
Methyl 3-[4-(cyclopropylamino)-1-ethyl-1H-pyrazolo[3,4-b]pyridin-5-yl]-5-(2-methoxy-2-oxoethyl)-4,5-dihydroisoxazole-5-carboxylate (Compound No. 12);

Yield: 83%

m/z: (M⁺+1) 402.2.
Methyl 3-[4-(cyclopentylamino)-1,3-dimethyl-1H-pyrazolo[3,4-b]pyridin-5-yl]-5-methyl-4,5-dihydroisoxazole-5-carboxylate (Compound No. 13);

Yield: 61.4%

m/z: (M⁺+1) 372.1.
Methyl 3-[4-(cyclopentylamino)-1,3-dimethyl-1H-pyrazolo[3,4-b]pyridin-5-yl]-5-(2-methoxy-2-oxoethyl)-4,5-dihydroisoxazole-5-carboxylate (Compound No. 14);

Yield: 60%

m/z: (M41) 430.0.
Methyl 3-[4-(cyclopropylamino)-1,3-dimethyl-1H-pyrazolo[3,4-b]pyridin-5-yl]-5-(2-methoxy-2-oxo ethyl)-4,5-dihydroisoxazole-5-carboxylate (Compound No. 15);

Yield: 30.6%

m/z: (M⁺+1) 402.0
Methyl 3-[4-(cyclopentylamino)-1-ethyl-1H-pyrazolo[3,4-b]pyridin-5-yl]-5-methyl-4,5-dihydroisoxazole-5-carboxylate (Compound No. 16),

Yield: 42%

m/z: (M⁺+1) 372.1.
Methyl 3-[4-(cyclopropylamino)-1,3-dimethyl-1H-pyrazolo[3,4-b]pyridin-5-yl]-5-methyl-4,5-dihydroisoxazole-5-carboxylate (Compound No. 17);

Yield: 15.3%

m/z: (M 41) 344.0.
Methyl 3-[4-(cyclopentylamino)-1-ethyl-1H-pyrazolo[3,4-b]pyridin-5-yl]-5-(2-methoxy-2-oxoethyl)-4,5-dihydroisoxazole-5-carboxylate (Compound No. 18);

Yield: 51%

m/z: (M 41) 430.1.

The following compounds can be prepared similarly:
Methyl 3-{4-[(1,1-dioxidotetrahydro-2H-thiopyran-4-yl)amino]-1-ethyl-1H-pyrazolo[3,4-b]pyridin-5-yl}-5-(2-methoxy-2-oxo ethyl)-4,5-dihydroisoxazole-5-carboxylate (Compound No. 151);
(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl) dimethanol (Compound No. 41);
(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)dimethanol (Compound No. 47);
{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}dimethanol (Compound No. 48);
{3-[4-(B enzylamino)-1-ethyl-1H-pyrazolo[3,4-b]pyridin-5-yl]-4,5-dihydroisoxazole-5,5-diyl}dimethanol
tert-Butyl 3-({5-[5,5-bis(hydroxymethyl)-4,5-dihydroisoxazol-3-yl]-1-ethyl-1H-pyrazolo[3,4-b]pyridin-4-yl}amino)cyclobutanecarboxylate (Compound No. 152); and
Methyl 3-[4-(b enzylamino)-1-ethyl-1H-pyrazolo[3,4-b]pyridin-5-yl]-5-(2-methoxy-2-oxoethyl)-4,5-dihydroisoxazole-5-carboxylate

### Example 9a

### Preparation of methyl 3-(4-amino-1-ethyl-1H-pyrazolo[3,4-b]pyridin-5-yl)-5-(2-methoxy-2-oxoethyl)-4,5-dihydroisoxazole-5-carboxylate

Palladium hydroxide/carbon (1 gm) is added to a solution of methyl 3-[4-(benzylamino)-1-ethyl-1H-pyrazolo[3,4-b]pyridin-5-yl]-5-(2-methoxy-2-oxoethyl)-4,5-dihydroisoxazole-5-carboxylate (1 gm, 0.0022 mole) (example 9) in methanol and the reaction mixture is stirred under hydrogen balloon for about 12 hours. It is filtered through a bed of celite and residue is washed with methanol. The combined filtrate is concentrated under reduced pressure to get the title compound.

### Example 9b

### Preparation of methyl 3-[1-ethyl-4-(pyridin-4-ylamino)-1H-pyrazolo[3,4-b]pyridin-5-yl]-5-(2-methoxy-2-oxoethyl)-4,5-dihydroisoxazole-5-carboxylate (Compound No. 153)

2,2′-Bis(diphenylphosphino)-1,1′-binaphthyl (0.3 equivalent), palladium acetate (0.09 equivalent) and cesium carbonate (1.5 equivalent) is added to 4-bromo pyridine (1 equivalent) in anhydrous dioxane under inert atmosphere. Methyl 3-(4-amino-1-ethyl-1H-pyrazolo[3,4-b]pyridin-5-yl)-5-(2-methoxy-2-oxoethyl)-4,5-dihydroisoxazole-5-carboxylate (1.3 equivalent) (example 9a) is added and the reaction mixture is stirred at reflux for about 10-12 hours. It is cooled to room temperature and filtered through celite. The reaction mixture is extracted with ethyl acetate. The organic layer is washed with water, dried over anhydrous sodium sulfate and concentrated in vacuo. The crude compound is purified by column chromatography.

### Example 10

### Preparation of 5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 6)

Lithium hydroxide monohydrate (75 mg, 0.00180 mole) in water (2 ml) was added to the solution of methyl 3-[4-(cyclohexylamino)-1-ethyl-1H-pyrazolo[3,4-b]pyridin-5-yl]-5-(2-methoxy-2-oxoethyl)-4,5-dihydroisoxazole-5-carboxylate (200 mg. 0.00045 mole) (example 9) in tetrahydrofuran (10 ml). The reaction mixture was stirred at room temperature overnight. The solvent was removed under reduced pressure, water was added and extraction was done with ethyl acetate. Aqueous layer was acidified by dilute hydrochloride, extracted with ethyl acetate, washed with brine, dried over anhydrous sodium sulfate and concentrated under vacuum to give white solid compound.

Yield: 120 mg (64%)

m/z: (M 41) 415.96

NMR: (δ, CDCl₃): 1.24-2.04 (m, 13H), 3.02 (s, 2H), 3.76-3.80 (d, 1H), 4.04-4.09 (m, 2H), 4.36-4.42 (q, 2H), 8.15-8.20 (d, 2H), 8.64-8.66 (d, 1H).

The following compounds were prepared similarly:
5-(Carboxymethyl)-3-[4-(cyclopropylamino)-1-ethyl-1H-pyrazolo[3,4-b]pyridin-5-yl]-4,5-dihydroisoxazole-5-carboxylic acid (Compound No. 19);

Yield: 45%

m/z: (M 41) 374.2.
3-[4-(Cyclopropylamino)-1-ethyl-1H-pyrazolo[3,4-b]pyridin-5-yl]-5-methyl-4,5-dihydroisoxazole-5-carboxylic acid (Compound No. 20);

Yield: 62.5%

m/z: (M41) 330.0.

### Example 11

### Preparation of 2-{3-[4-(cyclohexylamino)-1-ethyl-1H-pyrazolo[3,4-b]pyridin-5-yl]-5-(hydroxymethyl)-4,5-dihydroisoxazol-5-yl}ethanol (Compound No. 7)

Sodium borohydride (14 mg, 0.00036 mole) was added to the solution of methyl 3-[4-(cyclohexylamino)-1-ethyl-1H-pyrazolo[3,4-b]pyridin-5-yl]-5-(2-methoxy-2-oxoethyl)-4,5-dihydroisoxazole-5-carboxylate (80 mg, 0.00018 mole) (example 9) in tetrahydrofuran (5 ml), then methanol (2 drops) was added and the reaction mixture was stirred at room temperature overnight. It was quenched with saturated ammonium chloride solution, diluted with ethyl acetate, extracted with brine, dried over anhydrous sodium sulfate and concentrated in vacuo. The crude product obtained was purified by column chromatography.

Yield: 70 mg (98%)

m/z: (M 41) 388.28

NMR: (δ, CDCl₃): 1.36-2.11 (m, 15H), 3.37-3.60 (m, 2H), 3.74-3.95 (m, 5H), 4.44-4.47 (q, 2H), 7.97 (s, 1H), 8.13 (s, 1H), 8.75-8.77 (d, 1H).

The following compounds were prepared similarly:
{3-[4-(Cyclopropylamino)-1-ethyl-1H-pyrazolo[3,4-b]pyridin-5-yl]-5-methyl-4,5-dihydroisoxazol-5-yl}methanol (Compound No. 21);

Yield: 26.4%

m/z: (M41) 316.2.
2-[3-[4-(Cyclopropylamino)-1-ethyl-1H-pyrazolo[3,4-b]pyridin-5-yl]-5-(hydroxymethyl)-4,5-dihydroisoxazol-5-yl]ethanol (Compound No. 22);

Yield: 83.4%.

m/z: (M41) 346.3.
{3-[4-(Cyclopentylamino)-1,3-dimethyl-1H-pyrazolo[3,4-b]pyridin-5-yl]-5-methyl-4,5-dihydroisoxazol-5-yl}methanol (Compound No. 23);

Yield: 25.2%

m/z: (M 41) 344.1.
2-[3-[4-(Cyclopentylamino)-1,3-dimethyl-1H-pyrazolo[3,4-b]pyridin-5-yl]-5-(hydroxymethyl)-4,5-dihydroisoxazol-5-yl]ethanol (Compound No. 24);

Yield: 90%

m/z: (M41) 374.2.
{3-[4-(Cyclopropylamino)-1,3-dimethyl-1H-pyrazolo[3,4-b]pyridin-5-yl]-5-methyl-4,5-dihydroisoxazol-5-yl}methanol (Compound No. 25);

Yield: 15.62%)

m/z: (M 41) 316.1.
{3-[4-(Cyclopentylamino)-1-ethyl-1H-pyrazolo[3,4-b]pyridin-5-yl]-5-methyl-4,5-dihydroisoxazol-5-yl}methanol (Compound No. 26);

Yield: 63.9%

m/z: (M 41) 344.2.

### Example 12

### Preparation of 5-(2-Amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 8)

Aqueous ammonia (2 ml) was added to the solution of methyl 3-[4-(cyclohexylamino)-1-ethyl-1H-pyrazolo[3,4-b]pyridin-5-yl]-5-(2-methoxy-2-oxoethyl)-4,5-dihydroisoxazole-5-carboxylate (52 mg, 0.00017 mole) (example 9) in tetrahydrofuran (5 ml). The reaction mixture was stirred at room temperature for about 3 hours. After completion of the reaction, the solvent was evaporated off and water was added. Solid, which precipitated out was filtered and dried under vacuum.

Yield: 21 mg (43%)

m/z: (M 41) 414.30

NMR: (δ, CDCl₃): 1.25-2.16 (m, 13H), 2.94-3.15 (m, 2H), 3.77-3.94 (m, 3H), 4.44-4.49 (q, 2H), 7.97 (s, 1H), 8.09 (s, 1H), 8.54-8.56 (d, 1H).

The following compounds were prepared similarly:
3-[4-(Cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 9);

Yield: 38%

m/z: (M⁺+1) 442.26
5-(2-Amino-2-oxoethyl)-3-[4-(cyclopropylamino)-1-ethyl-1H-pyrazolo[3,4-b]pyridin-5-yl]-4,5-dihydroisoxazole-5-carboxamide (Compound No. 27);

Yield: 86%

m/z: (M 41) 372.2.
3-[4-(Cyclopropylamino)-1-ethyl-1H-pyrazolo[3,4-b]pyridin-5-yl]-5-methyl-4,5-dihydroisoxazole-5-carboxamide (Compound No. 28);

Yield: 83.6%

m/z: (M 41) 329.1.
3-[4-(Cyclopentylamino)-1,3-dimethyl-1H-pyrazolo[3,4-b]pyridin-5-yl]-N, 5-dimethyl-4,5-dihydroisoxazole-5-carboxamide (Compound No. 29);

Yield: 57.2%

m/z: (M⁺+1) 371.1.
3-[4-(Cyclopentylamino)-1,3-dimethyl-1H-pyrazolo[3,4-b]pyridin-5-yl]-5-methyl-4,5-dihydroisoxazole-5-carboxamide (Compound No. 30),

Yield: 44.5%

m/z: (M⁺+1) 357.1.
5-(2-Amino-2-oxoethyl)-3-[4-(cyclopentylamino)-1,3-dimethyl-1H-pyrazolo[3,4-b]pyridin-5-yl]-4,5-dihydroisoxazole-5-carboxamide (Compound No. 31);

Yield: 85.8%

m/z: (M⁺+1) 400.1.
N-cyclopropyl-3-[4-(cyclopropylamino)-1,3-dimethyl-1H-pyrazolo[3,4-b]pyridin-5-yl]-5-[2-(cyclopropylamino)-2-oxo ethyl]-4,5-dihydroisoxazole-5-carboxamide (Compound No. 32);

Yield: 63.82%.

m/z: (M⁺+1) 474.1.
5-(2-Amino-2-oxoethyl)-3-[4-(cyclopropylamino)-1,3-dimethyl-1H-pyrazolo[3,4-b]pyridin-5-yl]-4,5-dihydroisoxazole-5-carboxamide (Compound No. 33);

Yield: 86.2%

m/z: (M 41) 372.0

The following compounds can be prepared similarly:
5-(2-Amino-2-oxoethyl)-3-{4-[(1,1-dioxidotetrahydro-2H-thiopyran-4-yl)amino]-1-ethyl-1H-pyrazolo[3,4-b]pyridin-5-yl}-4,5-dihydroisoxazole-5-carboxamide (Compound No. 40);
5-(2-Amino-2-oxoethyl)-3-{4-[(1,1-dioxidotetrahydro-2H-thiopyran-4-yl)amino]-1-ethyl-3-methyl-1H-pyrazolo[3,4-b]pyridin-5-yl}-4,5-dihydroisoxazole-5-carboxamide (Compound No. 44);
3-{4-[(1,1-Dioxidotetrahydro-2H-thiopyran-4-yl)amino]-1-ethyl-1H-pyrazolo[3,4-b]pyridin-5-yl}-N-methyl-5-[2-(methylamino)-2-oxoethyl]-4,5-dihydroisoxazole-5-carboxamide (Compound No. 45);
3-{4-[(1,1-Dioxidotetrahydro-2H-thiopyran-4-yl)amino]-1-ethyl-3-methyl-1H-pyrazolo[3,4-b]pyridin-5-yl}-N-methyl-5-[2-(methylamino)-2-oxoethyl]-4,5-dihydroisoxazole-5-carboxamide (Compound No. 46);
5-(2-Amino-2-oxoethyl)-3-[1-ethyl-3-methyl-4-(tetrahydro-2H-pyran-4-ylamino)-1H-pyrazolo[3,4-b]pyridin-5-yl]-4,5-dihydroisoxazole-5-carboxamide (Compound No. 65);
3-[1-Ethyl-3-methyl-4-(tetrahydro-2H-pyran-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 66);
5-(2-Amino-2-oxoethyl)-3-[1-ethyl-4-(tetrahydro-2H-pyran-4-ylamino)-1H-pyrazolo[3,4-b]pyridin-5-yl]-4,5-dihydroisoxazole-5-carboxamide (Compound No. 67);
3-[1-Ethyl-4-(tetrahydro-2H-pyran-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 68);
5-(2-Amino-2-oxoethyl)-3-[4-(cyclohexylamino)-1-ethyl-3-methyl-1H-pyrazolo[3,4-b]pyridin-5-yl]-4,5-dihydroisoxazole-5-carboxamide (Compound No. 69);
3-[4-(Cyclohexylamino)-1-ethyl-3-methyl-1H-pyrazolo[3,4-b]pyridin-5-yl]-N-methyl-5-[2-(methylamino)-2-oxoethyl]-4,5-dihydroisoxazole-5-carboxamide (Compound No. 70);
5-(2-Amino-2-oxoethyl)-3-[1-ethyl-4-(pyridin-3-ylamino)-1H-pyrazolo[3,4-b]pyridin-5-yl]-4,5-dihydroisoxazole-5-carboxamide (Compound No. 129);
5-(2-Amino-2-oxoethyl)-3-[1-ethyl-4-(pyrazin-2-ylamino)-1H-pyrazolo[3,4-b]pyridin-5-yl]-4,5-dihydroisoxazole-5-carboxamide (Compound No. 130);
5-(2-Amino-2-oxoethyl)-3-[1-ethyl-4-(pyrimidin-2-ylamino)-1H-pyrazolo[3,4-b]pyridin-5-yl]-4,5-dihydroisoxazole-5-carboxamide (Compound No. 131);
5-(2-Amino-2-oxoethyl)-3-[1-ethyl-4-(1,2,4-triazin-5-ylamino)-1H-pyrazolo[3,4-b]pyridin-5-yl]-4,5-dihydroisoxazole-5-carboxamide (Compound No. 132);
5-(2-Amino-2-oxoethyl)-3-[1-ethyl-4-(pyrimidin-5-ylamino)-1H-pyrazolo[3,4-b]pyridin-5-yl]-4,5-dihydroisoxazole-5-carboxamide (Compound No. 133);
5-(2-Amino-2-oxoethyl)-3-[1-ethyl-4-(1,3-thiazol-4-ylamino)-1H-pyrazolo[3,4-b]pyridin-5-yl]-4,5-dihydroisoxazole-5-carboxamide (Compound No. 134);
5-(2-Amino-2-oxoethyl)-3-[1-ethyl-4-(4H-1,2,4-triazol-4-ylamino)-1H-pyrazolo[3,4-b]pyridin-5-yl]-4,5-dihydroisoxazole-5-carboxamide (Compound No. 135);
5-(2-Amino-2-oxoethyl)-3-[1-ethyl-4-(pyridin-4-ylamino)-1H-pyrazolo[3,4-b]pyridin-5-yl]-4,5-dihydroisoxazole-5-carboxamide (Compound No. 136);
5-(2-Amino-2-oxoethyl)-3-[1-ethyl-4-(2H-tetrazol-5-ylamino)-1H-pyrazolo[3,4-b]pyridin-5-yl]-4,5-dihydroisoxazole-5-carboxamide (Compound No. 137);
5-(2-Amino-2-oxoethyl)-3-[1-ethyl-4-(1H-tetrazol-5-ylamino)-1H-pyrazolo[3,4-b]pyridin-5-yl]-4,5-dihydroisoxazole-5-carboxamide (Compound No. 138);
5-(2-Amino-2-oxoethyl)-3-[1-ethyl-4-(furan-3-ylamino)-1H-pyrazolo[3,4-b]pyridin-5-yl]-4,5-dihydroisoxazole-5-carboxamide (Compound No. 139);
3-[1-Ethyl-4-(pyridin-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 140);
3-[1-Ethyl-4-(pyrazin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 141);
3-[1-Ethyl-4-(pyrimidin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 142);
3-[1-Ethyl-4-(1,2,4-triazin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 143);
3-[1-Ethyl-4-(pyrimidin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 144);
3-[1-Ethyl-4-(1,3-thiazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 145);
3-[1-Ethyl-4-(4H-1,2,4-triazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 146);
3-[1-Ethyl-4-(pyridin-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 147);
3-[1-Ethyl-4-(2H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 148);
3-[1-Ethyl-4-(1H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 149); and
3-[1-Ethyl-4-(furan-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 150).

### Example 13

### Preparation of (5S^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 34)

Step a: 5-(Carboxymethyl)-3-[4-(cyclohexylamino)-1-ethyl-1H-pyrazolo[3,4-b]pyridin-5-yl]-4,5-dihydroisoxazole-5-carboxylic acid (1 gm, 0.0024 mole) (example 10) and L-ephedrine (870 mg, 0.0053 mole) in ethyl acetate (20 ml) were refluxed for about 4 hours. The reaction mixture was slowly brought to 35° C. and kept as such for 18 hours. The solid crystallized was filtered off under nitrogen, washed with acetone and dried under vacuum.

Yield: 750 mg

Step b: Product from step a (750 mg) was taken in water (20 ml) and concentrated hydrochloric acid (2 drops) was added. The reaction mixture was stirred for about 3 hours. It was extracted with ethyl acetate, washed with brine and concentrated.

Yield: 350 mg (35%).

m/z: (M 41) 415.96

Chiral purity: 99.56%

The following compound was prepared similarly:
(5R^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 35)

Yield: 35%

m/z: (M41) 415.96

Chiral purity: 99.67%

### Example 14

### Preparation of (5R^{$})-5-(2-amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 37)

(5R^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (50 mg, 0.000124 mole) (example 13) was dissolved in dimethylformamide (2.5 ml). At 0° C., hydroxybenzotriazole (66 mg, 0.000492 mole) and ammonium carbonate (13 mg, 0.000135 mole) were added under argon atmosphere and reaction mixture was stirred for about 1 hour at 0° C. 1-Ethyl-3-(3-dimethylaminopropyl) carbodiimide hydrochloride (51 mg, 0.000270 mole) was then added to the reaction mixture and it was stirred at 0° C. overnight. The reaction mixture was diluted with water and extracted with ethyl acetate. Organic layer was dried over anhydrous sodium sulfate and concentrated under reduced pressure. The crude compound was purified by preparative thin layer chromatography.

Yield: 40 mg (80.38%)

m/z: (M41) 414.39

Chiral purity: 96.63%

The following compound was prepared similarly:
(5S^{$})-5-(2-amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 36)

m/z: (M 41) 414.39

Chiral purity: 98.56%

### Example 15

### Preparation of (5S^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 38)

Triethyl amine (0.048 ml, 0.00034 mole) was added to methylamine hydrochloride (23 mg, 0.000345 mole) taken in dimethylformamide (1 ml) at 0° C. and the reaction mixture was stirred for about 10 minutes. At 0° C., (58⁵)-5-(carboxymethyl)-3-[4-(cyclohexylamino)-1-ethyl-1H-pyrazolo[3,4-b]pyridin-5-yl]-4,5-dihydroisoxazole-5-carboxylic acid (70 mg, 0.000172 mole) (example 13) and hydroxybenzotriazole (93 mg, 0.00069 mole) were added under argon atmosphere and reaction mixture was stirred for about 1 hour at 0° C. 1-Ethyl-3-(3-dimethylaminopropyl) carbodiimide hydrochloride (72 mg, 0.000380 mole) was added to the reaction mixture and it was stirred at room temperature overnight. Water was added and the extraction was done with ethyl acetate. Organic layer was dried over anhydrous sodium sulfate and concentrated under reduced pressure. The crude compound was purified by preparative thin layer chromatography.

Yield: 30 mg (40%)

m/z: (M 41) 442.36

Chiral purity: 99.13%

The following compound was prepared similarly:
(5R^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 39)

m/z: (M 41) 442.36

Chiral purity: 99.0%

### Example 16

### Preparation of 3-{4-[(1,1-dioxidotetrahydro-2H-thiopyran-4-yl)amino]-1-ethyl-1H-pyrazolo[3,4-b]pyridin-5-yl}isoxazole-5,5(4H)-dicarboxylic acid (Compound No. 154)

(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl) dimethanol (1 equivalent) (example 9) is taken in acetone and potassium permanganate (6 equivalent) in water is added and the reaction mixture is heated at refluxing temperature for about 3-4 hours. The solvent is evaporated off. The residue is acidified to pH of about 3-4 and the precipitated solid is filtered and dried under vacuo.

### Example 17

### Preparation of 2,2′-(3-{4-[(1,1-dioxidotetrahydro-2H-thiopyran-4-yl)amino]-1-ethyl-1H-pyrazolo[3,4-b]pyridin-5-yl}-4,5-dihydroisoxazole-5,5-diyl)diacetic acid (Compound No. 155)

3-{4-[(1,1-dioxidotetrahydro-2H-thiopyran-4-yl)amino]-1-ethyl-1H-pyrazolo[3,4-b]pyridin-5-yl}isoxazole-5,5(4H)-dicarboxylic acid (example 16) (0.023 mole) in thionyl chloride and dimethylformamide is heated at 80° C. for about 1.5 hours. Excess thionyl chloride is distilled out completely to get 3-{4-[(1,1-dioxidotetrahydro-2H-thiopyran-4-yl)amino]-1-ethyl-1H-pyrazolo[3,4-b]pyridin-5-yl}isoxazole-5,5 (4H)-dicarbonyl dichloride. This intermediate is slowly added to a solution of trimethylsilyldiazomethane (0.056 mole) and triethylamine (0.056 mole) in acetonitrile: tetrahydrofuran at 0° C. The reaction mixture is stirred at the same temperature for about 2 hours. The volatiles are removed under reduced pressure and the mixture is dried under high vacuum to get diazoketo intermediate. Water: dioxane mixture is added to this intermediate and mixture is heated at 70° C. Freshly prepared silver oxide (8.0 gm) is added portion wise over a period of 30 minutes and then the reaction mixture is refluxed for about 1.5 hours. It is filtered through celite. The filter pad is washed with dioxane. The filtrate is concentrated and residue obtained is purified through column chromatography.

### Example 18

### Preparation of 2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)diacetamide (Compound No. 51)

The title compound is prepared by following the procedure of example 15 using 2,2′-(3-{4-[(1,1-dioxidotetrahydro-2H-thiopyran-4-yl)amino]-1-ethyl-1H-pyrazolo[3,4-b]pyridin-5-yl}-4,5-dihydroisoxazole-5,5-diyl)diacetic acid (example 17).

The following compounds can be prepared similarly:
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-methylacetamide) (Compound No. 52);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-methylacetamide) (Compound No. 53);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)diacetamide (Compound No. 54);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-ethylacetamide) (Compound No. 55);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-ethylacetamide) (Compound No. 56);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}diacetamide (Compound No. 57);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}diacetamide (Compound No. 58);
2,2′-{3-[4-(Cyclohexylamino)-1-ethyl-3-methyl-1H-pyrazolo[3,4-b]pyridin-5-yl]-4,5-dihydroisoxazole-5,5-diyl}diacetamide (Compound No. 59);
2,2′-{3-[4-(Cyclohexylamino)-1-ethyl-1H-pyrazolo[3,4-b]pyridin-5-yl]-4,5-dihydroisoxazole-5,5-diyl}diacetamide (Compound No. 60);
2,2′-{3-[4-(Cyclohexylamino)-1-ethyl-1H-pyrazolo[3,4-b]pyridin-5-yl]-4,5-dihydroisoxazole-5,5-diyl}bis(N-methylacetamide) (Compound No. 61);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 62);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 63);
2,2′-{3-[4-(Cyclohexylamino)-1-ethyl-3-methyl-1H-pyrazolo[3,4-b]pyridin-5-yl]-4,5-dihydroisoxazole-5,5-diyl}bis(N-methylacetamide) (Compound No. 64);

3-[4-(Cyclohexylamino)-1-ethyl-1H-pyrazolo[3,4-b]pyridin-5-yl]isoxazole-5,5(4H)-dicarboxamide (Compound No. 83);
3-[1-Ethyl-4-(tetrahydro-2H-pyran-4-ylamino)-1H-pyrazolo[3,4-b]pyridin-5-yl]isoxazole-5,5(4H)-dicarboxamide (Compound No. 84);
3-{1-Ethyl-4-[(4-oxocyclohexyl)amino]-1H-pyrazolo[3,4-b]pyridin-5-yl}isoxazole-5,5(4H)-dicarboxamide (Compound No. 85);
3-{1-Ethyl-4-[(4-oxocyclohexyl)amino]-1H-pyrazolo[3,4-b]pyridin-5-yl}-N,N′-dimethylisoxazole-5,5(4H)-dicarboxamide (Compound No. 86);
N,N′-diethyl-3-{1-ethyl-4-[(4-oxocyclohexyl)amino]-1H-pyrazolo[3,4-b]pyridin-5-yl}isoxazole-5,5(4H)-dicarboxamide (Compound No. 87);
3-[4-(Cyclohexylamino)-1-ethyl-1H-pyrazolo[3,4-b]pyridin-5-yl]-N,N′-dimethylisoxazole-5,5(4H)-dicarboxamide (Compound No. 88);
3-[1-Ethyl-4-(tetrahydro-2H-pyran-4-ylamino)-1H-pyrazolo[3,4-b]pyridin-5-yl]-N,N′-dimethylisoxazole-5,5(4H)-dicarboxamide (Compound No. 89);
N,N′-diethyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 90);
3-[4-(Cyclohexylamino)-1-ethyl-1H-pyrazolo[3,4-b]pyridin-5-yl]-N,N′-diethylisoxazole-5,5(4H)-dicarboxamide (Compound No. 91);
N,N′-dicyclobutyl-3-{1-ethyl-4-[(4-oxocyclohexyl)amino]-1H-pyrazolo[3,4-b]pyridin-5-yl}isoxazole-5,5(4H)-dicarboxamide (Compound No. 92);
N,N′-dicyclobutyl-3-[4-(cyclohexylamino)-1-ethyl-1H-pyrazolo[3,4-b]pyridin-5-yl]isoxazole-5,5(4H)-dicarboxamide (Compound No. 93);
N,N′-dicyclobutyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 94);
N,N′-dicyclopentyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 95);
3-[4-(Cyclohexylamino)-1-ethyl-1H-pyrazolo[3,4-b]pyridin-5-yl]-N,N′-dicyclopentylisoxazole-5,5(4H)-dicarboxamide (Compound No. 96);
N,N′-dicyclopentyl-3-{1-ethyl-4-[(4-oxocyclohexyl)amino]-1H-pyrazolo[3,4-b]pyridin-5-yl}isoxazole-5,5(4H)-dicarboxamide (Compound No. 97);
N,N′-dicyclohexyl-3-{1-ethyl-4-[(4-oxocyclohexyl)amino]-1H-pyrazolo[3,4-b]pyridin-5-yl}isoxazole-5,5(4H)-dicarboxamide (Compound No. 98);
N,N′-dicyclohexyl-3-[4-(cyclohexylamino)-1-ethyl-1H-pyrazolo[3,4-b]pyridin-5-yl]isoxazole-5,5(4H)-dicarboxamide (Compound No. 99);
N,N′-dicyclohexyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 100);
3-[1-Ethyl-4-(tetrahydro-2H-pyran-4-ylamino)-1H-pyrazolo[3,4-b]pyridin-5-yl]-N,N′-bis(1-methylcyclohexyl)isoxazole-5,5(4H)-dicarboxamide (Compound No. 101);
3-[4-(Cyclohexylamino)-1-ethyl-1H-pyrazolo[3,4-b]pyridin-5-yl]-N,N′-bis(1-methylcyclohexyl)isoxazole-5,5(4H)-dicarboxamide (Compound No. 102);
3-{1-Ethyl-4-[(4-oxocyclohexyl)amino]-1H-pyrazolo[3,4-b]pyridin-5-yl}-N,N′-bis(1-methylcyclohexyl)isoxazole-5,5(4H)-dicarboxamide (Compound No. 103);
3-{1-Ethyl-4-[(4-oxocyclohexyl)amino]-1H-pyrazolo[3,4-b]pyridin-5-yl}-N,N′-bis(pyridin-4-ylmethyl)isoxazole-5,5(4H)-dicarboxamide (Compound No. 104);
3-[4-(Cyclohexylamino)-1-ethyl-1H-pyrazolo[3,4-b]pyridin-5-yl]-N,N′-bis(pyridin-4-ylmethyl)isoxazole-5,5(4H)-dicarboxamide (Compound No. 105);
3-[1-Ethyl-4-(tetrahydro-2H-pyran-4-ylamino)-1H-pyrazolo[3,4-b]pyridin-5-yl]-N,N′-bis(pyridin-4-ylmethyl)isoxazole-5,5(4H)-dicarboxamide (Compound No. 106);
3-[1-Ethyl-4-(tetrahydro-2H-pyran-4-ylamino)-1H-pyrazolo[3,4-b]pyridin-5-yl]-N,N′-bis(4-fluorophenyl)isoxazole-5,5(4H)-dicarboxamide (Compound No. 107);
3-[4-(Cyclohexylamino)-1-ethyl-1H-pyrazolo[3,4-b]pyridin-5-yl]-N,N′-bis(4-fluorophenyl)isoxazole-5,5(4H)-dicarboxamide (Compound No. 108);
3-{1-Ethyl-4-[(4-oxocyclohexyl)amino]-1H-pyrazolo[3,4-b]pyridin-5-yl}-N,N′-bis(4-fluorophenyl)isoxazole-5,5(4H)-dicarboxamide (Compound No. 109)
4-({5-[5,5-Bis(pyrrolidin-1-ylcarbonyl)-4,5-dihydroisoxazol-3-yl]-1-ethyl-1H-pyrazolo[3,4-b]pyridin-4-yl}amino)cyclohexanone (Compound No. 110);
5-[5,5-Bis(pyrrolidin-1-ylcarbonyl)-4,5-dihydroisoxazol-3-yl]-N-cyclohexyl-1-ethyl-1H-pyrazolo[3,4-b]pyridin-4-amine (Compound No. 111);
5-[5,5-Bis(pyrrolidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 112);
5-[5,5-Bis(piperidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 113);
5-[5,5-Bis(piperidin-1-ylcarbonyl)-4,5-dihydroisoxazol-3-yl]-N-cyclohexyl-1-ethyl-1H-pyrazolo[3,4-b]pyridin-4-amine (Compound No. 114);
4-({5-[5,5-Bis(piperidin-1-ylcarbonyl)-4,5-dihydroisoxazol-3-yl]-1-ethyl-1H-pyrazolo[3,4-b]pyridin-4-yl}amino)cyclohexanone (Compound No. 115)
4-{[5-(5,5-Bis {[4-(hydroxymethyl)piperidin-1-yl]carbonyl}-4,5-dihydroisoxazol-3-yl)-1-ethyl-1H-pyrazolo[3,4-b]pyridin-4-yl]amino}cyclohexanone (Compound No. 116);
4-[(5-{5,5-Bis[(4-methylpiperazin-1-yl)carbonyl]-4,5-dihydroisoxazol-3-yl}-1-ethyl-1H-pyrazolo[3,4-b]pyridin-4-yl)amino]cyclo hexanone (Compound No. 117);
5-{5,5-Bis[(4-methylpiperazin-1-yl)carbonyl]-4,5-dihydroisoxazol-3-yl}-N-cyclohexyl-1-ethyl-1H-pyrazolo[3,4-b]pyridin-4-amine (Compound No. 118);
5-{5,5-Bis[(4-methylpiperazin-1-yl)carbonyl]-4,5-dihydroisoxazol-3-yl}-1-ethyl-N-(tetrahydro-2H-pyran-4-yl)-1H-pyrazolo[3,4-b]pyridin-4-amine (Compound No. 119);
5-[5,5-Bis(piperazin-1-yl carbonyl)-4,5-dihydroisoxazol-3-yl]-N-cyclohexyl-1-ethyl-1H-pyrazolo[3,4-b]pyridin-4-amine (Compound No. 120);
4-({5-[5,5-Bis(piperazin-1-ylcarbonyl)-4,5-dihydroisoxazol-3-yl]-1-ethyl-1H-pyrazolo[3,4-b]pyridin-4-yl}amino)cyclohexanone (Compound No. 121);
5-[5,5-Bis(piperazin-1-yl carbonyl)-4,5-dihydroisoxazol-3-yl]-1-ethyl-N-(tetrahydro-2H-pyran-4-yl)-1H-pyrazolo[3,4-b]pyridin-4-amine (Compound No. 122);
5-{5,5-Bis[(3-benzyl-3,6-diazabicyclo[3.1.0]hex-6-yl)carbonyl]-4,5-dihydroisoxazol-3-yl}-N-cyclohexyl-1-ethyl-1H-pyrazolo[3,4-b]pyridin-4-amine (Compound No. 123);
5-{5,5-Bis[(3-benzyl-3,6-diazabicyclo[3.1.0] hex-6-yl)carbonyl]-4,5-dihydroisoxazol-3-yl}-1-ethyl-N-(tetrahydro-2H-pyran-4-yl)-1H-pyrazolo[3,4-b]pyridin-4-amine (Compound No. 124)
4-[(5-{5,5-Bis[(3-benzyl-3,6-diazabicyclo [3.1.0]hex-6-yl)carbonyl]-4,5-dihydroisoxazol-3-yl}-1-ethyl-1H-pyrazolo[3,4-b]pyridin-4-yl)amino]cyclohexanone (Compound No. 125);
N,N′-bis(3-benzyl-3-azabicyclo [3.1.0] hex-6-yl)-3-{1-ethyl-4-[(4-oxocyclohexyl)amino]-1H-pyrazolo[3,4-b]pyridin-5-yl} isoxazole-5,5(4H)-dicarboxamide (Compound No. 126);
N,N′-bis(3-benzyl-3-azabicyclo [3.1.0] hex-6-yl)-3-[4-(cyclohexylamino)-1-ethyl-1H-pyrazolo[3,4-b]pyridin-5-yl]isoxazole-5,5(4H)-dicarboxamide (Compound No. 127); and
N,N′-bis(3-benzyl-3-azabicyclo [3.1.0] hex-6-yl)-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 128).

### Example 19

### Preparation of 3-({5-[5,5-Bis(hydroxymethyl)-4,5-dihydroisoxazol-3-yl]-1-ethyl-1H-pyrazolo[3,4-b]pyridin-4-yl}amino)cyclobutanecarboxylic acid (Compound No. 49)

Trifluoroacetic acid (4 equivalent) is added to the solution of tert-butyl 3-({5-[5,5-bis(hydroxymethyl)-4,5-dihydroisoxazol-3-yl]-1-ethyl-1H-pyrazolo[3,4-b]pyridin-4-yl}amino)cyclobutanecarboxylate (1 equivalent) (example 9) in dichloroethane and the reaction mixture is stirred at room temperature for about 2 hours under inert atmosphere. It is cooled and diluted with ethyl acetate. The organic layer is washed with saturated sodium bicarbonate, water and brine, dried over anhydrous sodium sulfate and concentrated under reduced pressure to get the title compound.

The following compounds can be prepared similarly:
4-({5-[5,5-Bis(hydroxymethyl)-4,5-dihydroisoxazol-3-yl]-1-ethyl-1H-pyrazolo[3,4-b]pyridin-4-yl}amino)cyclohexanecarboxylic acid (Compound No. 42);
4-({5-[5,5-Bis(hydroxymethyl)-4,5-dihydroisoxazol-3-yl]-1-ethyl-3-methyl-1H-pyrazolo[3,4-b]pyridin-4-yl}amino)cyclohexanecarboxylic acid (Compound No. 43);
3-({5-[5,5-Bis(hydroxymethyl)-4,5-dihydroisoxazol-3-yl]-1-ethyl-3-methyl-1H-pyrazolo[3,4-b]pyridin-4-yl}amino)cyclobutanecarboxylic acid (Compound No. 50);
4-({5-[5,5-Bis(2-amino-2-oxo ethyl)-4,5-dihydroisoxazol-3-yl]-1-ethyl-1H-pyrazolo[3,4-b]pyridin-4-yl}amino)cyclohexanecarboxylic acid (Compound No. 71);
4-({5-[5,5-Bis(2-amino-2-oxo ethyl)-4,5-dihydroisoxazol-3-yl]-1-ethyl-3-methyl-1H-pyrazolo[3,4-b]pyridin-4-yl}amino)cyclohexanecarboxylic acid (Compound No. 72);
4-[(5-{5,5-Bis[2-(methylamino)-2-oxo ethyl]-4,5-dihydroisoxazol-3-yl}-1-ethyl-3-methyl-1H-pyrazolo[3,4-b]pyridin-4-yl)amino]cyclohexanecarboxylic acid (Compound No. 73);
4-[(5-{5,5-Bis[2-(methylamino)-2-oxo ethyl]-4,5-dihydroisoxazol-3-yl}-1-ethyl-1H-pyrazolo[3,4-b]pyridin-4-yl)amino]cyclohexanecarboxylic acid (Compound No. 74);
4-[(5-{5,5-Bis[2-(ethylamino)-2-oxo ethyl]-4,5-dihydroisoxazol-3-yl}-1-ethyl-1H-pyrazolo[3,4-b]pyridin-4-yl)amino]cyclohexanecarboxylic acid (Compound No. 75);
4-[(5-{5,5-Bis[2-(ethylamino)-2-oxoethyl]-4,5-dihydroisoxazol-3-yl}-1-ethyl-3-methyl-1H-pyrazolo[3,4-b]pyridin-4-yl)amino]cyclohexanecarboxylic acid (Compound No. 76);
4-[(1-Ethyl-5-{5[2-(ethylamino)-2-oxoethyl]-5-(ethylcarbamoyl)-4,5-dihydroisoxazol-3-yl}-3-methyl-1H-pyrazolo[3,4-b]pyridin-4-yl)amino]cyclohexanecarboxylic acid (Compound No. 77);
4-[(1-Ethyl-5-{5-[2-(ethylamino)-2-oxoethyl]-5-(ethylcarbamoyl)-4,5-dihydroisoxazol-3-yl}-1H-pyrazolo[3,4-b]pyridin-4-yl)amino]cyclohexanecarboxylic acid (Compound No. 78);
4-[(1-Ethyl-5-{5-[2-(methylamino)-2-oxoethyl]-5-(methylcarbamoyl)-4,5-dihydroisoxazol-3-yl}-1H-pyrazolo[3,4-b]pyridin-4-yl)amino]cyclohexanecarboxylic acid (Compound No. 79);
4-[(1-Ethyl-3-methyl-5-{5-[2-(methylamino)-2-oxoethyl]-5-(methylcarbamoyl)-4,5-dihydroisoxazol-3-yl}-1H-pyrazolo[3,4-b]pyridin-4-yl)amino]cyclohexanecarboxylic acid (Compound No. 80);
4-({5-[5-(2-Amino-2-oxoethyl)-5-carbamoyl-4,5-dihydroisoxazol-3-yl]-1-ethyl-3-methyl-1H-pyrazolo[3,4-b]pyridin-4-yl}amino)cyclohexanecarboxylic acid (Compound No. 81); and
4-({5-[5-(2-Amino-2-oxoethyl)-5-carbamoyl-4,5-dihydroisoxazol-3-yl]-1-ethyl-1H-pyrazolo[3,4-b]pyridin-4-yl}amino)cyclohexanecarboxylic acid (Compound No. 82).

### Example 20

### Preparation of 4-({5-[5,5-bis(hydroxymethyl)-4,5-dihydroisoxazol-3-yl]-1-ethyl-1H-pyrazolo[3,4-b]pyridin-4-yl}amino)cyclohexanone (Compound No. 156)

4-({5-[5,5-Bis(hydroxymethyl)-4,5-dihydroisoxazol-3-yl]-1-ethyl-1H-pyrazolo[3,4-b]pyridin-4-yl}amino)cyclohexanol (0.251 mmol) (example 9) is dissolved in dichloromethane and the reaction mixture is cooled upto 5° C. Pyridinium chlorochromate (0.502 mmol) is added and the reaction mixture is stirred for about 5 minutes at the same temperature. It is warmed to room temperature and stirred at room temperature for about 16 hours. Dilution is done with dichloromethane and filtration is done using celite. The organic layers are combined, concentrated and purified by preparative thin layer chromatography.

### Example 21

### Efficacy of Compounds

(a)(i) PDE4B Enzyme Assay

The efficacy of compounds as PDE4 inhibitors was determined by an enzyme assay using cell lysate of HEK293 cells transfected with PDE4B2 plasmids as PDE4B source. The enzyme reaction was carried out in the presence of cAMP (1 μM) at 30° C. in the presence or absence of test compound for 45-60 minutes. An aliquot of this reaction mixture was taken further for the ELISA assay and the protocol of the kit followed to determine level of cAMP in the sample. The concentration of the cAMP in the sample directly correlated with the degree of PDE4 enzyme inhibition. Results were expressed as percent control and the IC₅₀ values of test compounds were reported. IC₅₀ values of test compounds were found to be in the range of 1 nM to 10 μM concentration.

(a)(ii) PDE7 Enzyme Assay

The efficacy of compounds as PDE7 inhibitors was determined by an enzyme assay using recombinant human PDE7A enzyme (J. Med. Chem., 43, (2000), 683-689). The enzyme reaction was carried out in the presence of cAMP (1 μM) at 37° C. in the presence or absence of test compound for 60 minutes. An aliquot of this reaction mixture was taken further for the ELISA assay and the protocol of the kit was followed to determine level of cAMP in the sample. The concentration of the cAMP in the sample directly correlated with the degree of PDE7 enzyme inhibition. Results were expressed as percent control and the IC₅₀ values of test compounds, calculated using Graph pad prism, were found to be in the range of lower 7 nM to 10 μM concentration.

### (b) Cell Based Assay for TNF-α Release

Method of Isolation of Human Peripheral Blood Mononuclear Cells (PBMNC's)

Human whole blood was collected in vacutainer tubes containing heparin or EDTA as an anti coagulant. The blood was diluted (1:1) in sterile phosphate buffered saline and 10 ml was carefully layered over 5 ml Ficoll Hypaque gradient (density 1.077 g/ml) in a 15 ml conical centrifuge tube. The sample was centrifuged at 3000 rpm for 25 minutes in a swing-out rotor at room temperature. After centrifugation, interface of cells were collected, diluted at least 1:5 with PBS (phosphate buffered saline) and washed three times by centrifugation at 2500 rpm for 10 minutes at room temperature. The cells were resuspended in serum free RPMI 1640 medium at a concentration of 2 million cells/ml.

### LPS (Lipopolysaccharide) Stimulation of Human PBMNC's

PBMN cells (0.1 ml; 2 million/ml) were co-incubated with 20 μl of compound (final DMSO concentration of 0.2%) for 10 minutes in a flat bottom 96 well microtiter plate. Compounds were dissolved in DMSO initially and diluted in medium for a final concentration of 0.2% DMSO. LPS (1 μg/ml, final concentration) was then added at a volume of 10 μl per well. After 30 minutes, 20 μl of fetal calf serum (final concentration of 10%) was added to each well. Cultures were incubated overnight at 37° C. in an atmosphere of 5% CO₂ and 95% air. Supernatant were then removed and tested by ELISA for TNF-α release using a commercial kit (e.g. BD Biosciences). For whole blood, the plasma samples were diluted 1:20 for ELISA. The level of TNF-α in treated wells was compared with the vehicle (0.2% DMSO in RPMI medium) treated controls and inhibitory potency of compound was expressed as IC₅₀ values calculated by using Graph pad prism. IC₅₀ values of test compounds were found to be in the range of 30 nM to 10 μM concentration.

$$Percent inhibition = 100 - \frac{Percent TNF - α drug treated}{Percent TNF - α in vehicle treated} \times 100$$

(c) In-Vitro Assay to Evaluate Efficacy of Compounds in Combination with p38 MAP Kinase Inhibitors

Perform the assay as described in (b) above, with individual compounds and their combinations tested at sub-optimal doses.

(d) In-Vitro Assay to Evaluate Efficacy of Compounds in Combination with β2-Agonists
Measurement of Intracellular cAMP Elevation in U937 Cells

Grow U937 cells (human promonocytic cell line) in endotoxin-free RPMI 1640+HEPES medium containing 10% (v/v) heat-inactivated foetal bovine serum and 1% (v/v) of an antibiotic solution (5000 IU/ml penicillin, 5000 μg/ml streptomycin). Resuspend cells (0.25×10⁶/200 μl) in Krebs' buffer solution and incubate at 37° C. for 15 minutes in the presence of test compounds or vehicle (0.2% DMSO in RPMI medium). Initiate generation of cAMP by adding 50 μl of 10 μM prostaglandin (PGE2). Stop the reaction after 15 minutes, by adding 1 N HCl (50 μl) and place on ice for 30 minutes. Centrifuge the sample (450 g, 3 minutes), and measure levels of cAMP in the supernatant using cAMP enzyme-linked immunosorbent assay kit (Assay Designs). Calculate percent inhibition by the following formula and calculate IC₅₀ value using Graph pad prism.

$$Percent inhibition = 100 - \frac{Percent conversion in drug treated}{Percent conversion in vehicle treated} \times 100$$

We claim:

1. A compound having the structure of Formula I: 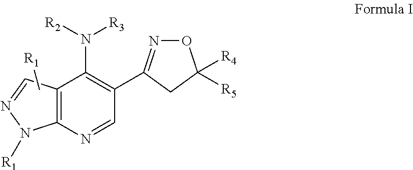
or its pharmaceutically acceptable salts, wherein R₁ is hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ and R₃ independently are hydrogen, aryl, heteroaryl, 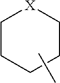
or 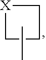
wherein X is CH₂, CO, O, CH(CH₂)ₙ(OH), CH(COOR_{f}), or S(O)ₙ, (wherein n is an integer from 0-2 and R_{f} is hydrogen, alkyl, alkenyl, cycloalkyl, aryl, aralkyl, heterocyclyl, heteroaryl, (heterocyclyl)alkyl or (heteroaryl)alkyl); and
R₄ and R₅ independently are alkyl, —CN, —(CH₂)ₙC(═O)NR_{f}R_{q} {wherein n is an integer from 0-2 and R_{f} and R_{q} independently are hydrogen, alkyl, alkenyl, cycloalkyl, aryl, aralkyl, heterocyclyl, heteroaryl, (heterocyclyl)alkyl, (heteroaryl)alkyl or R_{f} and R_{q} taken together with the nitrogen atom to which they are attached form a optionally substituted heterocyclyl ring}, —(CH₂)ₙC(═O)OR_{f} {wherein n and R_{f} are the same as defined above} or —(CH₂)ₙ₁OR_{f} {wherein n1 is an integer from 0-3 and R_{f} is the same as defined above}.

2. A compound which is selected from
{3-[4-(Cyclohexylamino)-1-ethyl-1H-pyrazolo[3,4-b]pyridin-5-yl]-4,5-dihydroisoxazole-5,5-diyl}dimethanol (Compound No. 1);
Methyl 3-[4-(cyclohexylamino)-1-ethyl-1H-pyrazolo[3,4-b]pyridin-5-yl]-5-(2-methoxy-2-oxoethyl)-4,5-dihydroisoxazole-5-carboxylate (Compound No. 2);
Methyl 3-[1-ethyl-4-(tetrahydro-2H-pyran-4-ylamino)-1H-pyrazolo[3,4-b]pyridin-5-yl]-5-(2-methoxy-2-oxoethyl)-4,5-dihydroisoxazole-5-carboxylate (Compound No. 3);
{3-[1-Ethyl-4-(tetrahydro-2H-pyran-4-ylamino)-1H-pyrazolo[3,4-b]pyridin-5-yl]-4,5-dihydroisoxazole-5,5-diyl}dimethanol (Compound No. 4);
4-({5-[5,5-Bis(hydroxymethyl)-4,5-dihydroisoxazol-3-yl]-1-ethyl-1H-pyrazolo[3,4-b]pyridin-4-yl}amino)cyclohexanol (Compound No. 5);
5-(Carboxymethyl)-3-[4-(cyclohexylamino)-1-ethyl-1H-pyrazolo[3,4-b]pyridin-5-yl]-4,5-dihydroisoxazole-5-carboxylic acid (Compound No. 6);
2-{3-[4-(Cyclohexylamino)-1-ethyl-1H-pyrazolo[3,4-b]pyridin-5-yl]-5-(hydroxymethyl)-4,5-dihydroisoxazol-5-yl}ethanol (Compound No. 7);
5-(2-Amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 8);
3-[4-(Cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 9);
(5S^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 34);
(5R^{$})-5-(carboxymethyl)-3-[4-(cyclohexylamino)-1-ethyl-1H-pyrazolo[3,4-b]pyridin-5-yl]-4,5-dihydroisoxazole-5-carboxylic acid (Compound No. 35);
(5S^{$})-5-(2-amino-2-oxoethyl)-3-[4-(cyclohexylamino)-1-ethyl-1H-pyrazolo[3,4-b]pyridin-5-yl]-4,5-dihydroisoxazole-5-carboxamide (Compound No. 36);
(5R^{$})-5-(2-amino-2-oxo ethyl)-3-[4-(cyclohexylamino)-1-ethyl-1H-pyrazolo[3,4-b]pyridin-5-yl]-4,5-dihydroisoxazole-5-carboxamide (Compound No. 37);
(5S^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 38);
(5R^{$})-3-[4-(cyclohexylamino)-1-ethyl-1H-pyrazolo[3,4-b]pyridin-5-yl]-N-methyl-5-[2-(methylamino)-2-oxoethyl]-4,5-dihydroisoxazole-5-carboxamide (Compound No. 39);
5-(2-Amino-2-oxoethyl)-3-{4-[(1,1-dioxidotetrahydro-2H-thiopyran-4-yl)amino]-1-ethyl-1H-pyrazolo[3,4-b]pyridin-5-yl}-4,5-dihydroisoxazole-5-carboxamide (Compound No. 40);
(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)dimethanol (Compound No. 41);
4-({5-[5,5-Bis(hydroxymethyl)-4,5-dihydroisoxazol-3-yl]-1-ethyl-1H-pyrazolo[3,4-b]pyridin-4-yl}amino)cyclohexanecarboxylic acid (Compound No. 42);
4-({5-[5,5-Bis(hydroxymethyl)-4,5-dihydroisoxazol-3-yl]-1-ethyl-3-methyl-1H-pyrazolo[3,4-b]pyridin-4-yl}amino)cyclohexanecarboxylic acid (Compound No. 43);
5-(2-Amino-2-oxoethyl)-3-{4-[(1,1-dioxidotetrahydro-2H-thiopyran-4-yl)amino]-1-ethyl-3-methyl-1H-pyrazolo[3,4-b]pyridin-5-yl}-4,5-dihydroisoxazole-5-carboxamide (Compound No. 44);
3-{4-[(1,1-Dioxidotetrahydro-2H-thiopyran-4-yl)amino]-1-ethyl-1H-pyrazolo[3,4-b]pyridin-5-yl}-N-methyl-5-[2-(methylamino)-2-oxoethyl]-4,5-dihydroisoxazole-5-carboxamide (Compound No. 45);
3-{4-[(1,1-Dioxidotetrahydro-2H-thiopyran-4-yl)amino]-1-ethyl-3-methyl-1H-pyrazolo[3,4-b]pyridin-5-yl}-N-methyl-5-[2-(methylamino)-2-oxo ethyl]-4,5-dihydroisoxazole-5-carboxamide (Compound No. 46);
(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl) dimethanol (Compound No. 47);
{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}dimethanol (Compound No. 48);
3-({5-[5,5-Bis(hydroxymethyl)-4,5-dihydroisoxazol-3-yl]-1-ethyl-1H-pyrazolo[3,4-b]pyridin-4-yl}amino)cyclobutanecarboxylic acid (Compound No. 49);
3-({5-[5,5-Bis(hydroxymethyl)-4,5-dihydroisoxazol-3-yl]-1-ethyl-3-methyl-1H-pyrazolo[3,4-b]pyridin-4-yl}amino)cyclobutanecarboxylic acid (Compound No. 50); 2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)diacetamide (Compound No. 51);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-methylacetamide) (Compound No. 52);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-methylacetamide) (Compound No. 53);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)diacetamide (Compound No. 54);
2,2′-(3-{4-[(1,1-Dioxidotetrahydro-2H-thiopyran-4-yl)amino]-1-ethyl-1H-pyrazolo[3,4-b]pyridin-5-yl}-4,5-dihydroisoxazole-5,5-diyl)bis(N-ethylacetamide) (Compound No. 55);
2,2′-(3-{4-[(1,1-Dioxidotetrahydro-2H-thiopyran-4-yl)amino]-1-ethyl-3-methyl-1H-pyrazolo[3,4-b]pyridin-5-yl}-4,5-dihydroisoxazole-5,5-diyl)bis(N-ethylacetamide) (Compound No. 56);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}diacetamide (Compound No. 57);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}diacetamide (Compound No. 58);
2,2′-{3-[4-(Cyclohexylamino)-1-ethyl-3-methyl-1H-pyrazolo[3,4-b]pyridin-5-yl]-4,5-dihydroisoxazole-5,5-diyl}diacetamide (Compound No. 59);
2,2′-{3-[4-(Cyclohexylamino)-1-ethyl-1H-pyrazolo[3,4-b]pyridin-5-yl]-4,5-dihydroisoxazole-5,5-diyl}diacetamide (Compound No. 60);
2,2′-{3-[4-(Cyclohexylamino)-1-ethyl-1H-pyrazolo[3,4-b]pyridin-5-yl]-4,5-dihydroisoxazole-5,5-diyl}bis(N-methylacetamide) (Compound No. 61);
2,2′-{3-[1-Ethyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 62);
2,2′-{3-[1-Ethyl-3-methyl-4-(tetrahydro-2H-pyran-4-ylamino)-1H-pyrazolo[3,4-b]pyridin-5-yl]-4,5-dihydroisoxazole-5,5-diyl}bis(N-methylacetamide) (Compound No. 63);
2,2′-{3-[4-(Cyclohexylamino)-1-ethyl-3-methyl-1H-pyrazolo[3,4-b]pyridin-5-yl]-4,5-dihydroisoxazole-5,5-diyl}bis(N-methylacetamide) (Compound No. 64);
5-(2-Amino-2-oxoethyl)-3-[1-ethyl-3-methyl-4-(tetrahydro-2H-pyran-4-ylamino)-1H-pyrazolo[3,4-b]pyridin-5-yl]-4,5-dihydroisoxazole-5-carboxamide (Compound No. 65);
3-[1-Ethyl-3-methyl-4-(tetrahydro-2H-pyran-4-ylamino)-1H-pyrazolo[3,4-b]pyridin-5-yl]-N-methyl-5-[2-(methylamino)-2-oxoethyl]-4,5-dihydroisoxazole-5-107 carboxamide (Compound No. 66);
5-(2-Amino-2-oxoethyl)-3-[1-ethyl-4-(tetrahydro-2H-pyran-4-ylamino)-1H-pyrazolo[3,4-b]pyridin-5-yl]-4,5-dihydroisoxazole-5-carboxamide (Compound No. 67);
3-[1-Ethyl-4-(tetrahydro-2H-pyran-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 68);
5-(2-Amino-2-oxoethyl)-3-[4-(cyclohexylamino)-1-ethyl-3-methyl-1H-pyrazolo[3,4-b]pyridin-5-yl]-4,5-dihydroisoxazole-5-carboxamide (Compound No. 69);
3-[4-(Cyclohexylamino)-1-ethyl-3-methyl-1H-pyrazolo[3,4-b]pyridin-5-yl]-N-methyl-5-[2-(methylamino)-2-oxoethyl]-4,5-dihydroisoxazole-5-carboxamide (Compound No. 70);
4-({5-[5,5-Bis(2-amino-2-oxoethyl)-4,5-dihydroisoxazol-3-yl]-1-ethyl-1H-pyrazolo[3,4-b]pyridin-4-yl}amino)cyclohexanecarboxylic acid (Compound No. 71);
4-({5-[5,5-Bis(2-amino-2-oxoethyl)-4,5-dihydroisoxazol-3-yl]-1-ethyl-3-methyl-1H-pyrazolo[3,4-b]pyridin-4-yl}amino)cyclohexanecarboxylic acid (Compound No. 72);
4-[(5-{5,5-Bis[2-(methylamino)-2-oxoethyl]-4,5-dihydroisoxazol-3-yl}-1-ethyl-3-methyl-1H-pyrazolo[3,4-b]pyridin-4-yl)amino]cyclohexanecarboxylic acid (Compound No. 73);
4-[(5-{5,5-Bis[2-(methylamino)-2-oxoethyl]-4,5-dihydroisoxazol-3-yl}-1-ethyl-1H-pyrazolo[3,4-b]pyridin-4-yl)amino]cyclohexanecarboxylic acid (Compound No. 74);
4-[(5-{5,5-Bis[2-(ethylamino)-2-oxoethyl]-4,5-dihydroisoxazol-3-yl}-1-ethyl-1H-pyrazolo[3,4-b]pyridin-4-yl)amino]cyclohexanecarboxylic acid (Compound No. 75);
4-[(5-{5,5-Bis[2-(ethylamino)-2-oxoethyl]-4,5-dihydroisoxazol-3-yl}-1-ethyl-3-methyl-1H-pyrazolo[3,4-b]pyridin-4-yl)amino]cyclohexanecarboxylic acid (Compound No. 76);
4-[(1-Ethyl-5-{5-[2-(ethylamino)-2-oxoethyl]-5-(ethylcarbamoyl)-4, 5-dihydroisoxazol-3-yl}-3-methyl-1H-pyrazolo[3,4-b]pyridin-4-yl)amino]cyclohexanecarboxylic acid (Compound No. 77);
4-[(1-Ethyl-5-{5-[2-(ethylamino)-2-oxoethyl]-5-(ethylcarbamoyl)-4, 5-dihydroisoxazol-3-yl}-1H-pyrazolo[3,4-b]pyridin-4-yl)amino]cyclohexanecarboxylic acid (Compound No. 78);
4-[(1-Ethyl-5-{5-[2-(methylamino)-2-oxoethyl]-5-(methylcarbamoyl)-4, 5-dihydroisoxazol-3-yl}-1H-pyrazolo[3,4-b]pyridin-4-yl)amino]cyclohexanecarboxylic acid (Compound No. 79);
4-[(1-Ethyl-3-methyl-5-{5-[2-(methylamino)-2-oxoethyl]-5-(methylcarbamoyl)-4,5-dihydroisoxazol-3-yl}-1H-pyrazolo[3,4-b]pyridin-4-yl)amino]cyclohexanecarboxylic acid (Compound No. 80);
4-({5-[5-(2-Amino-2-oxoethyl)-5-carbamoyl-4,5-dihydroisoxazol-3-yl]-1-ethyl-3-methyl-1H-pyrazolo[3,4-b]pyridin-4-yl}amino)cyclohexanecarboxylic acid (Compound No. 81)
4-({5-[5-(2-Amino-2-oxoethyl)-5-carbamoyl-4,5-dihydroisoxazol-3-yl]-1-ethyl-1H-pyrazolo[3,4-b]pyridin-4-yl}amino)cyclohexanecarboxylic acid (Compound No. 82);
3-[4-(Cyclohexylamino)-1-ethyl-1H-pyrazolo[3,4-b]pyridin-5-yl]isoxazole-5,5(4H)-dicarboxamide (Compound No. 83);
3-[1-Ethyl-4-(tetrahydro-2H-pyran-4-ylamino)-1H-pyrazolo[3,4-b]pyridin-5-yl]isoxazole-5,5(4H)-dicarboxamide (Compound No. 84);
3-{1-Ethyl-4-[(4-oxocyclohexyl)amino]-1H-pyrazolo[3,4-b]pyridin-5-yl}isoxazole-5,5(4H)-dicarboxamide (Compound No. 85);
3-{1-Ethyl-4-[(4-oxocyclohexyl)amino]-1H-pyrazolo[3,4-b]pyridin-5-yl}-N,N′-dimethylisoxazole-5,5(4H)-dicarboxamide (Compound No. 86);
N,N′-diethyl-3-{1-ethyl-4-[(4-oxocyclohexyl)amino]-1H-pyrazolo[3,4-b]pyridin-5-yl}isoxazole-5,5(4H)-dicarboxamide (Compound No. 87);
3-[4-(Cyclohexylamino)-1-ethyl-1H-pyrazolo[3,4-b]pyridin-5-yl]-N,N′-dimethylisoxazole-5,5(4H)-dicarboxamide (Compound No. 88);
3-[1-Ethyl-4-(tetrahydro-2H-pyran-4-ylamino)-1H-pyrazolo[3,4-b]pyridin-5-yl]-N,N′-dimethylisoxazole-5,5(4H)-dicarboxamide (Compound No. 89);
N,N′-diethyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 90);
3-[4-(Cyclohexylamino)-1-ethyl-1H-pyrazolo[3,4-b]pyridin-5-yl]-N,N′-diethylisoxazole-5,5(4H)-dicarboxamide (Compound No. 91);
N,N′-dicyclobutyl-3-{1-ethyl-4-[(4-oxocyclohexyl)amino]-1H-pyrazolo[3,4-b]pyridin-5-yl}isoxazole-5,5(4H)-dicarboxamide (Compound No. 92);
N,N′-dicyclobutyl-3-[4-(cyclohexylamino)-1-ethyl-1H-pyrazolo[3,4-b]pyridin-5-yl]isoxazole-5,5(4H)-dicarboxamide (Compound No. 93);
N,N′-dicyclobutyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 94);
N,N′-dicyclopentyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 95);
3-[4-(Cyclohexylamino)-1-ethyl-1H-pyrazolo[3,4-b]pyridin-5-yl]-N,N′-dicyclopentylisoxazole-5,5(4H)-dicarboxamide (Compound No. 96);
N,N′-dicyclopentyl-3-{1-ethyl-4-[(4-oxocyclohexyl)amino]-1H-pyrazolo[3,4-b]pyridin-5-yl}isoxazole-5,5(4H)-dicarboxamide (Compound No. 97);
N,N′-dicyclohexyl-3-{1-ethyl-4-[(4-oxocyclohexyl)amino]-1H-pyrazolo[3,4-b]pyridin-5-yl}isoxazole-5,5(4H)-dicarboxamide (Compound No. 98);
N,N′-dicyclohexyl-3-[4-(cyclohexylamino)-1-ethyl-1H-pyrazolo[3,4-b]pyridin-5-yl]isoxazole-5,5(4H)-dicarboxamide (Compound No. 99);
N,N′-dicyclohexyl-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 100);
3-[1-Ethyl-4-(tetrahydro-2H-pyran-4-ylamino)-1H-pyrazolo[3,4-b]pyridin-5-yl]-N,N′-bis(1-methylcyclohexyl)isoxazole-5,5(4H)-dicarboxamide (Compound No. 101)
3-[4-(Cyclohexylamino)-1-ethyl-1H-pyrazolo[3,4-b]pyridin-5-yl]-N,N′-bis(1-methylcyclohexyl)isoxazole-5,5(4H)-dicarboxamide (Compound No. 102);
3-{1-Ethyl-4-[(4-oxocyclohexyl)amino]-1H-pyrazolo[3,4-b]pyridin-5-yl}-N,N′-bis(1-methylcyclohexyl)isoxazole-5,5(4H)-dicarboxamide (Compound No. 103);
3-{1-Ethyl-4-[(4-oxocyclohexyl)amino]-1H-pyrazolo[3,4-b]pyridin-5-yl}-N,N′-bis(pyridin-4-ylmethyl)isoxazole-5,5(4H)-dicarboxamide (Compound No. 104);
3-[4-(Cyclohexylamino)-1-ethyl-1H-pyrazolo[3,4-b]pyridin-5-yl]-N,N′-bis(pyridin-4-ylmethyl)isoxazole-5,5(4H)-dicarboxamide (Compound No. 105);
3-[1-Ethyl-4-(tetrahydro-2H-pyran-4-ylamino)-1H-pyrazolo[3,4-b]pyridin-5-yl]-N,N′-bis(pyridin-4-ylmethyl)isoxazole-5,5(4H)-dicarboxamide (Compound No. 106);
3-[1-Ethyl-4-(tetrahydro-2H-pyran-4-ylamino)-1H-pyrazolo[3,4-b]pyridin-5-yl]-N,N′-bis(4-fluorophenyl)isoxazole-5,5(4H)-dicarboxamide (Compound No. 107);
3-[4-(Cyclohexylamino)-1-ethyl-1H-pyrazolo[3,4-b]pyridin-5-yl]-N,N′-bis(4-fluorophenyl)isoxazole-5,5(4H)-dicarboxamide (Compound No. 108);
3-{1-Ethyl-4-[(4-oxo cyclohexyl)amino]-1H-pyrazolo[3,4-b]pyridin-5-yl}-N,N′-bis(4-fluorophenyl)isoxazole-5,5(4H)-dicarboxamide (Compound No. 109);
4-({5-[5,5-Bis(pyrrolidin-1-yl carbonyl)-4,5-dihydroisoxazol-3-yl]-1-ethyl-1H-pyrazolo[3,4-b]pyridin-4-yl}amino)cyclohexanone (Compound No. 110);
5-[5,5-Bis(pyrrolidin-1-ylcarbonyl)-4,5-dihydroisoxazol-3-yl]-N-cyclohexyl-1-ethyl-1H-pyrazolo[3,4-b]pyridin-4-amine (Compound No. 111);
5-[5,5-Bis(pyrrolidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 112);
5-[5,5-Bis(piperidin-1-ylcarbonyl)-4,5-dihydroisoxazol-3-yl]-1-ethyl-N-(tetrahydro-2H-pyran-4-yl)-1H-pyrazolo[3,4-b]pyridin-4-amine (Compound No. 113);
5-[5,5-Bis(piperidin-1-ylcarbonyl)-4,5-dihydroisoxazol-3-yl]-N-cyclohexyl-1-ethyl-1H-pyrazolo[3,4-b]pyridin-4-amine (Compound No. 114);
4-({5-[5,5-Bis(piperidin-1-ylcarbonyl)-4,5-dihydroisoxazol-3-yl]-1-ethyl-1H-pyrazolo[3,4-b]pyridin-4-yl}amino)cyclohexanone (Compound No. 115);
4-{[5-(5,5-Bis {[4-(hydroxymethyl)piperidin-1-yl]carbonyl}-4,5-dihydroisoxazol-3-yl)-1-ethyl-1H-pyrazolo[3,4-b]pyridin-4-yl]amino}cyclohexanone (Compound No. 116);
4-[(5-{5,5-Bis[(4-methylpiperazin-1-yl)carbonyl]-4,5-dihydroisoxazol-3-yl}-1-ethyl-1H-pyrazolo[3,4-b]pyridin-4-yl)amino]cyclo hexanone (Compound No. 117);
5-{5,5-Bis[(4-methylpiperazin-1-yl)carbonyl]-4,5-dihydroisoxazol-3-yl}-N-cyclohexyl-1-ethyl-1H-pyrazolo[3,4-b]pyridin-4-amine (Compound No. 118);
5-{5,5-Bis[(4-methylpiperazin-1-yl)carbonyl]-4,5-dihydroisoxazol-3-yl}-1-ethyl-N-(tetrahydro-2H-pyran-4-yl)-1H-pyrazolo[3,4-b]pyridin-4-amine (Compound No. 119);
5-[5,5-Bis(piperazin-1-yl carbonyl)-4,5-dihydroisoxazol-3-yl]-N-cyclohexyl-1-ethyl-1H-pyrazolo[3,4-b]pyridin-4-amine (Compound No. 120);
4-({5-[5,5-Bis(piperazin-1-ylcarbonyl)-4,5-dihydroisoxazol-3-yl]-1-ethyl-1H-pyrazolo[3,4-b]pyridin-4-yl}amino)cyclohexanone (Compound No. 121);
5-[5,5-Bis(piperazin-1-yl carbonyl)-4,5-dihydroisoxazol-3-yl]-1-ethyl-N-(tetrahydro-2H-pyran-4-yl)-1H-pyrazolo[3,4-b]pyridin-4-amine (Compound No. 122)
5-{5,5-Bis[(3-benzyl-3,6-diazabicyclo [3.1.0] hex-6-yl)carbonyl]-4, 5-dihydroisoxazol-3-yl}-N-cyclohexyl-1-ethyl-1H-pyrazolo[3,4-b]pyridin-4-amine (Compound No. 123);
5-{5,5-Bis[(3-benzyl-3,6-diazabicyclo [3.1.0] hex-6-yl)carbonyl]-4, 5-dihydroisoxazol-3-yl}-1-ethyl-N-(tetrahydro-2H-pyran-4-yl)-1H-pyrazolo[3,4-b]pyridin-4-amine (Compound No. 124)
4-[(5-{5,5-Bis[(3-benzyl-3,6-diazabicyclo [3.1.0]hex-6-yl)carbonyl]-4, 5-dihydroisoxazol-3-yl}-1-ethyl-1H-pyrazolo[3,4-b]pyridin-4-yl)amino]cyclohexanone (Compound No. 125);
N,N′-bis(3-benzyl-3-azabicyclo[3.1.0]hex-6-yl)-3-{1-ethyl-4-[(4-oxocyclohexyl)amino]-1H-pyrazolo[3,4-b]pyridin-5-yl} isoxazole-5,5(4H)-dicarboxamide (Compound No. 126);
N,N′-bis(3-benzyl-3-azabicyclo [3.1.0]hex-6-yl)-3-[4-(cyclohexylamino)-1-ethyl-1H-pyrazolo[3,4-b]pyridin-5-yl]isoxazole-5,5(4H)-dicarboxamide (Compound No. 127);
N,N′-bis(3-benzyl-3-azabicyclo [3.1.0]hex-6-yl)-3-[1-ethyl-4-(tetrahydro-2H-pyran-4-ylamino)-1H-pyrazolo[3,4-b]pyridin-5-yl]isoxazole-5,5(4H)-dicarboxamide (Compound No. 128);
5-(2-Amino-2-oxoethyl)-3-[1-ethyl-4-(pyridin-3-ylamino)-1H-pyrazolo[3,4-b]pyridin-5-yl]-4,5-dihydroisoxazole-5-carboxamide (Compound No. 129);
5-(2-Amino-2-oxoethyl)-3-[1-ethyl-4-(pyrazin-2-ylamino)-1H-pyrazolo[3,4-b]pyridin-5-yl]-4,5-dihydroisoxazole-5-carboxamide (Compound No. 130);
5-(2-Amino-2-oxoethyl)-3-[1-ethyl-4-(pyrimidin-2-ylamino)-1H-pyrazolo[3,4-b]pyridin-5-yl]-4,5-dihydroisoxazole-5-carboxamide (Compound No. 131);
5-(2-Amino-2-oxoethyl)-3-[1-ethyl-4-(1,2,4-triazin-5-ylamino)-1H-pyrazolo[3,4-b]pyridin-5-yl]-4,5-dihydroisoxazole-5-carboxamide (Compound No. 132);
5-(2-Amino-2-oxoethyl)-3-[1-ethyl-4-(pyrimidin-5-ylamino)-1H-pyrazolo[3,4-b]pyridin-5-yl]-4,5-dihydroisoxazole-5-carboxamide (Compound No. 133);
5-(2-Amino-2-oxoethyl)-3-[1-ethyl-4-(1,3-thiazol-4-ylamino)-1H-pyrazolo[3,4-b]pyridin-5-yl]-4,5-dihydroisoxazole-5-carboxamide (Compound No. 134);
5-(2-Amino-2-oxoethyl)-3-[1-ethyl-4-(4H-1,2,4-triazol-4-ylamino)-1H-pyrazolo[3,4-b]pyridin-5-yl]-4,5-dihydroisoxazole-5-carboxamide (Compound No. 135);
5-(2-Amino-2-oxoethyl)-3-[1-ethyl-4-(pyridin-4-ylamino)-1H-pyrazolo[3,4-b]pyridin-5-yl]-4,5-dihydroisoxazole-5-carboxamide (Compound No. 136);
5-(2-Amino-2-oxoethyl)-3-[1-ethyl-4-(2H-tetrazol-5-ylamino)-1H-pyrazolo[3,4-b]pyridin-5-yl]-4,5-dihydroisoxazole-5-carboxamide (Compound No. 137);
5-(2-Amino-2-oxoethyl)-3-[1-ethyl-4-(1H-tetrazol-5-ylamino)-1H-pyrazolo[3,4-b]pyridin-5-yl]-4,5-dihydroisoxazole-5-carboxamide (Compound No. 138);
5-(2-Amino-2-oxoethyl)-3-[1-ethyl-4-(furan-3-ylamino)-1H-pyrazolo[3,4-b]pyridin-5-yl]-4,5-dihydroisoxazole-5-carboxamide (Compound No. 139);
3-[1-Ethyl-4-(pyridin-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 140);
3-[1-Ethyl-4-(pyrazin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 141);
3-[1-Ethyl-4-(pyrimidin-2-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 142);
3-[1-Ethyl-4-(1,2,4-triazin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 143);
3-[1-Ethyl-4-(pyrimidin-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 144);
3-[1-Ethyl-4-(1,3-thiazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 145);
3-[1-Ethyl-4-(4H-1,2,4-triazol-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 146);
3-[1-Ethyl-4-(pyridin-4-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 147);
3-[1-Ethyl-4-(2H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 148);
3-[1-Ethyl-4-(1H-tetrazol-5-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 149);
3-[1-Ethyl-4-(furan-3-ylamino)-1H-pyrazolo[3,4-b]pyridin-5-yl]-N-methyl-5-[2-(methylamino)-2-oxoethyl]-4,5-dihydroisoxazole-5-carboxamide (Compound No. 150);
Methyl 3-{4-[(1,1-dioxidotetrahydro-2H-thiopyran-4-yl)amino]-1-ethyl-1H-pyrazolo[3,4-b]pyridin-5-yl}-5-(2-methoxy-2-oxo ethyl)-4,5-dihydroisoxazole-5-carboxylate (Compound No. 151);
tert-Butyl 3-({5-[5,5-bis(hydroxymethyl)-4,5-dihydroisoxazol-3-yl]-1-ethyl-1H-pyrazolo[3,4-b]pyridin-4-yl}amino)cyclobutanecarboxylate (Compound No. 152);
Methyl 3-[1-ethyl-4-(pyridin-4-ylamino)-1H-pyrazolo[3,4-b]pyridin-5-yl]-5-(2-methoxy-2-oxoethyl)-4,5-dihydroisoxazole-5-carboxylate (Compound No. 153);
3-{4-[(1,1-Dioxidotetrahydro-2H-thiopyran-4-yl)amino]-1-ethyl-1H-pyrazolo[3,4-b]pyridin-5-yl}isoxazole-5,5(4H)-dicarboxylic acid (Compound No. 154);
2,2′-(3-{4-[(1,1-Dioxidotetrahydro-2H-thiopyran-4-yl)amino]-1-ethyl-1H-329 pyrazolo[3,4-b]pyridin-5-yl}-4,5-dihydroisoxazole-5,5-diyl)diacetic acid (Compound No. 155); and
4-({5-[5,5-Bis(hydroxymethyl)-4,5-dihydroisoxazol-3-yl]-1-ethyl-1H-pyrazolo[3,4-b]pyridin-4-yl}amino)cyclohexanone (Compound No. 156).
or its pharmaceutically acceptable salts, pharmaceutically acceptable solvates, stereoisomers, tautomers, racemates, regioisomers, geometric isomers, prodrugs, metabolites, polymorphs or N-oxides.

3. A pharmaceutical composition comprising a therapeutically effective amount of a compound as defined in claim 1 or 2 along with one or more of pharmaceutically acceptable carriers, excipients or diluents.

4. A pharmaceutical composition comprising a therapeutically effective amount of a compound of claim 1 or 2, along with one or more of pharmaceutically acceptable carriers, excipients or diluents and at least one other compound selected from β2-agonists, corticosteroids, leukotriene antagonists, 5-lipoxygenase inhibitors, chemokine inhibitors, p38 kinase inhibitors, anticholinergics, antiallergics, PAF (platelet activating factor) antagonists, EGFR (epidermal growth factor receptor) kinase inhibitors, muscarinic receptor antagonists or combination(s) thereof.

5. A method for treating, preventing, inhibiting or suppressing inflammatory diseases, CNS diseases or autoimmune diseases, in a mammal, comprising administering a therapeutically effective amount of a compound of claim 1 or 2 or a therapeutically effective amount of a pharmaceutical composition of claim 3 or 4.

6. A method for the treatment, prevention, inhibition or suppression of multiple sclerosis, AIDS, rejection of transplant, rheumatoid arthritis, bronchitis, chronic obstructive pulmonary disease (COPD), asthma, psoriasis, allergic rhinitis, shock, atopic dermatitis, Crohn's disease, adult respiratory distress syndrome (ARDS), eosinophilic granuloma, allergic conjunctivitis, osteoarthritis, colitis, pancreatitis, and cancer in a mammal comprising administering a therapeutically effective amount of a compound of claim 1 or 2 or a therapeutically effective amount of a pharmaceutical composition of claim 3 or 4.

7. The method according to claim 5 or 6, wherein the disease is mediated through phosphodiesterase type 4 and/or 7.

8. A method for the preparation of a compound of Formula I, 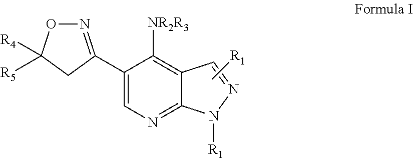
the method comprising,
(a) reacting a compound of Formula II with a compound of Formula III to give a compound of Formula IV, 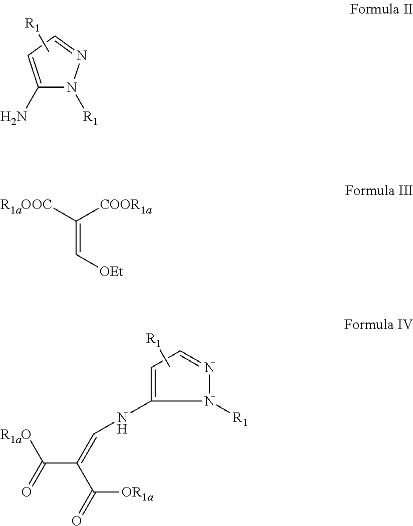
(b) heating the compound of Formula IV to give a compound of Formula Va, reacting the compound of Formula Va with phosphorous oxy halide to give a compound of Formula V,
or reacting the compound of Formula IV with phosphorous oxy halide to give a compound of Formula V, 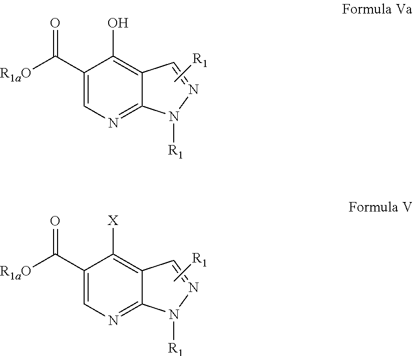
(c) reacting the compound of Formula V with a compound of Formula VI to give a compound of Formula VII,
hydrolyzing the compound of Formula VII to give a compound of Formula VIII, or hydrolyzing the compound of Formula V to give a compound of Formula VIIa, reacting the compound of Formula VIIa with a compound of Formula VI to give a compound of Formula VIII, 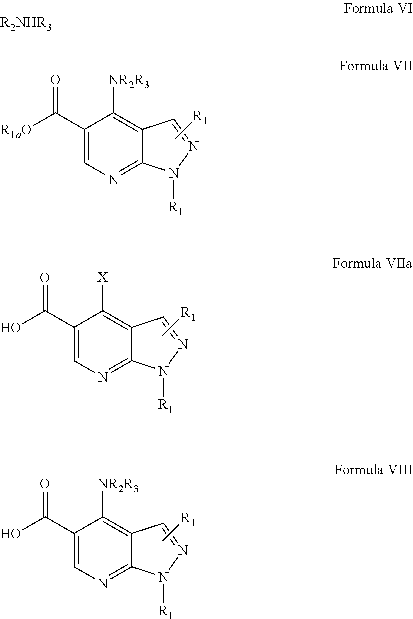
(d) reacting the compound of Formula VIII with a compound of Formula IX to give a compound of Formula X, 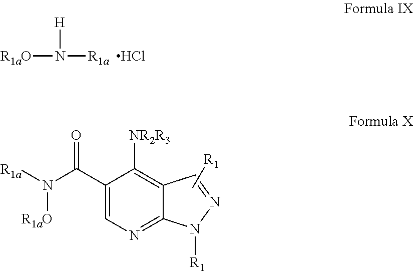
(e) reducing the compound of Formula X to give a compound of Formula XI, 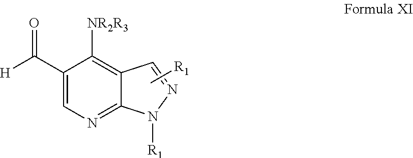
(f) reacting the compound of Formula XI with hydroxylamine hydrochloride to give a compound of Formula XII, 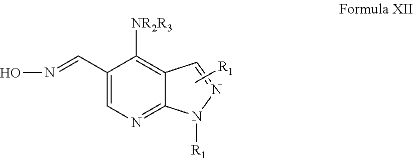
(g) reacting the compound of Formula XII with a compound of Formula XIII 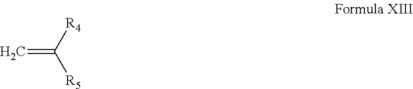
to give a compound of Formula I,
wherein R₁ₐ is alkyl, X is a halogen, and R₁, R₂, R₃, R₄ and R₅ are the same as defined in claim 1.

9. A method for the preparation of compounds of Formulae XV, XVa, XVI, XVIa, XVIII and XVIIIa, 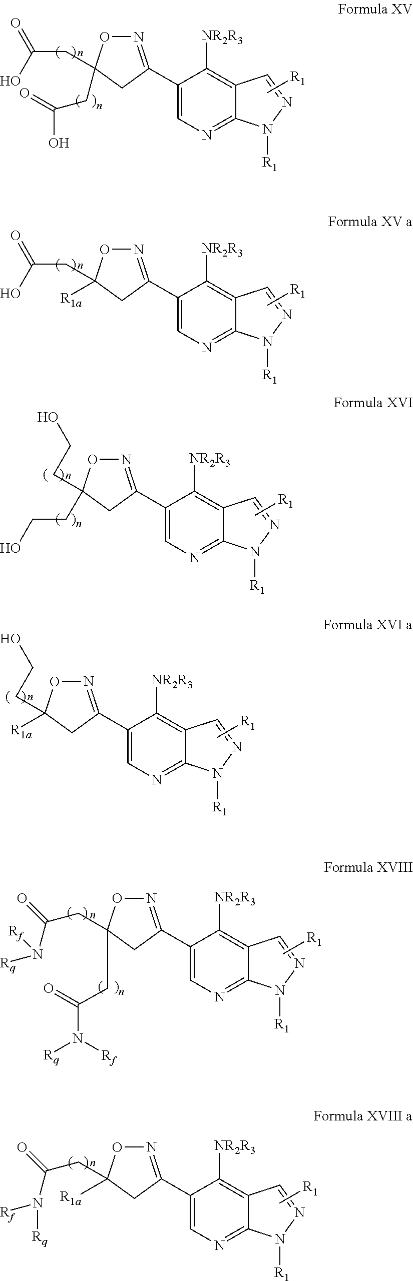
the method comprising,
(a) hydrolyzing a compound of Formula XIV or a compound of Formula XIVa 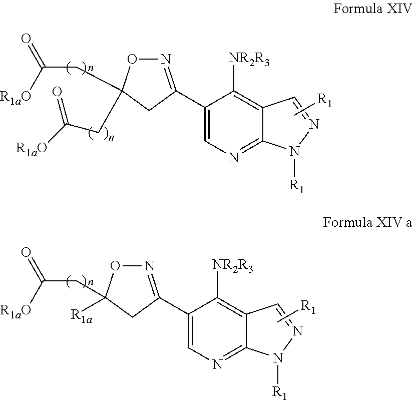
to give a compound of Formula XV or a compound of Formula XVa, respectively,
(b) reducing the compound of Formula XIV or the compound of Formula XIVa to give a compound of Formula XVI or a compound of Formula XVIa, respectively,
(c) reacting the compound of Formula XIV or the compound of Formula XIVa, with a compound of Formula XVII
R_{f}NHR_{q} Formula XVII
to give a compound of Formula XVIII or a compound of Formula XVIIIa, respectively,
wherein R₁ₐ is alkyl, n, R₁, R₂ and R₃, R_{f} and R_{q} are the same as defined in claim 1.

10. A method for the preparation of compounds of Formulae XXI, XXII and XXIII, 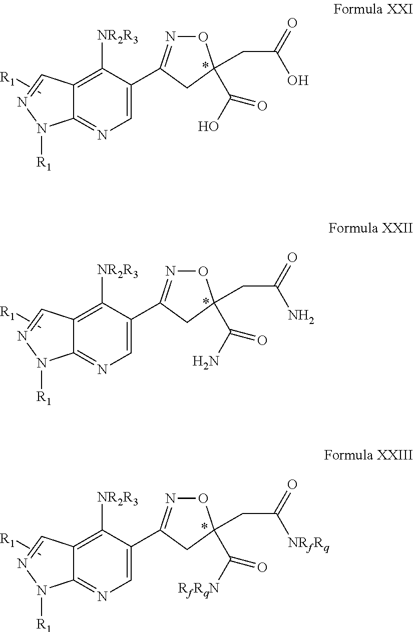
the method comprising,
(a) reacting a compound of Formula XVb with a chiral resolving agent, ‘Q’ of Formula XIX to give a compound of Formula XX, 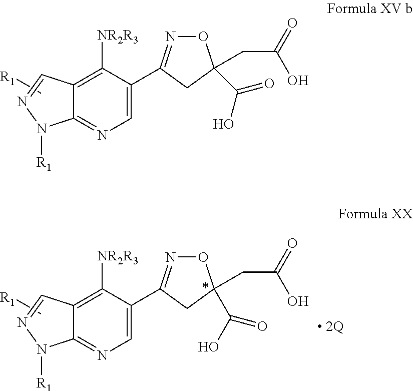
(b) hydrolyzing the compound of Formula XX to give a compound of Formula XXI,
(c) reacting the compound of Formula XXI with
(i) ammonium carbonate to give a compound of Formula XXII,
(ii) a compound of Formula XVII
R_{f}NHR_{q} Formula XVII
to give a compound of Formula XXIII,
wherein * represents a chiral centre and R₁, R₂, R₃, R_{f} and R_{q} are the same as defined in claim 1.

11. A method for the preparation of a compound of Formula XXX, 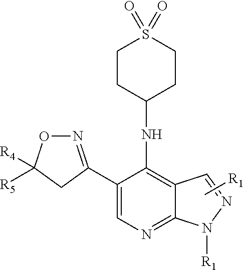
the method comprising,
(a) reacting a compound of Formula V with a compound of Formula VIa to give a compound of Formula XXIV, 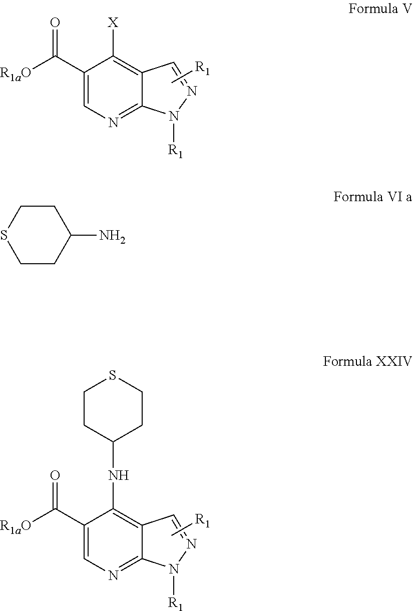
(b) oxidising the compound of Formula XXIV to give a compound of Formula XXV, 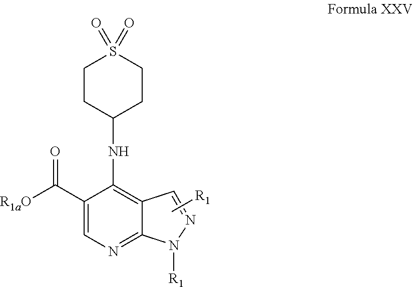
(c) hydrolyzing the compound of Formula XXV to give a compound of Formula XXVI, 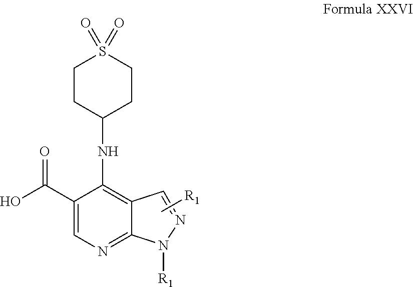
(d) reacting the compound of Formula XXVI with a compound of Formula IX to give a compound of Formula XXVII, 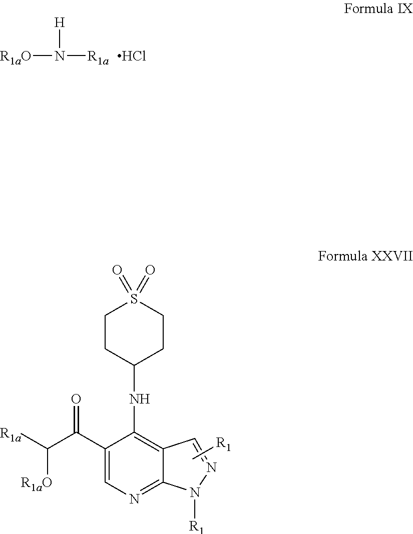
(e) reducing the compound of Formula XXVII to give a compound of Formula XXVIII, 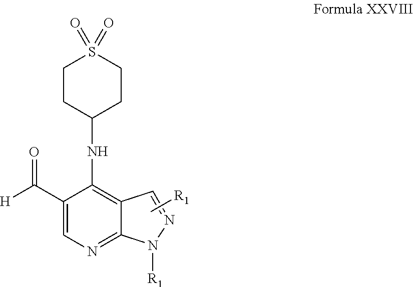
(f) reacting the compound of Formula XXVIII with hydroxylamine hydrochloride to give a compound of Formula XXIX, 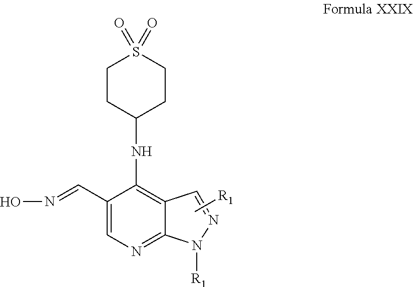
(g) reacting the compound of Formula XXIX with a compound of Formula XIII 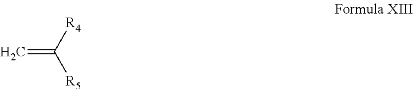
to give a compound of Formula XXX,
wherein X is halogen, R₁ₐ is alkyl and R₁, R₄ and R₅ are the same as defined in claim 1.

12. A method for the preparation of a compound of Formula XLVI, 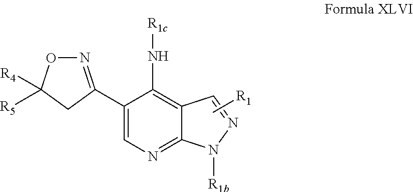
the method comprising
(a) heating a compound of Formula XXXI to give a compound of Formula XXXII, 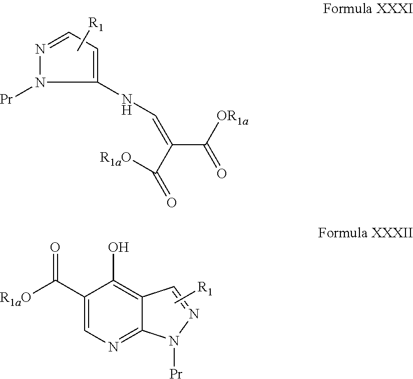
(b) reacting the compound of Formula XXXII with phosphorous oxy halide to give a compound of Formula XXXIII, 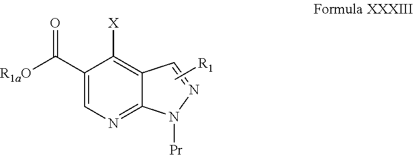
(c) reacting the compound of Formula XXXIII with a compound of Formula XXXIV to give a compound of Formula XXXV, 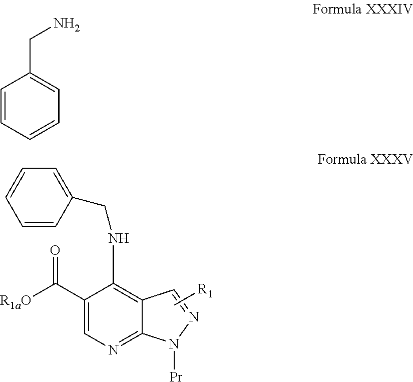
(d) hydrolyzing the compound of Formula XXXV to give a compound of Formula XXXVI, 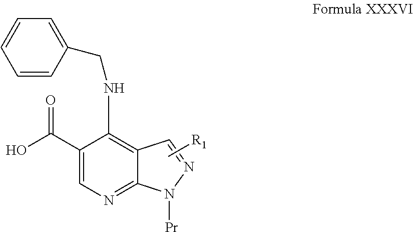
(e) reacting the compound of Formula XXXVI with a compound of Formula IX to give a compound of Formula XXXVII, 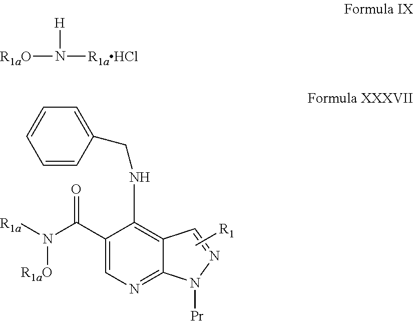
(f) deprotecting the compound of Formula XXXVII to give a compound of Formula XXXVIII, 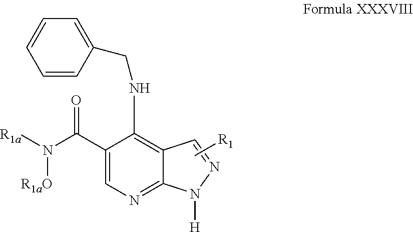
(g) reacting the compound of Formula XXXVIII with a compound of Formula XXXIX to give a compound of Formula XL, 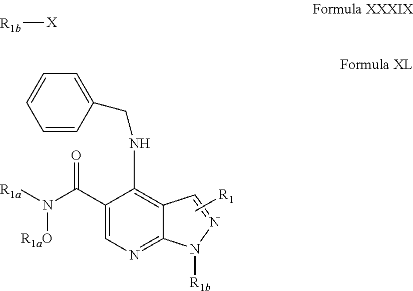
(h) reducing the compound of Formula XL to give a compound of Formula XLI, 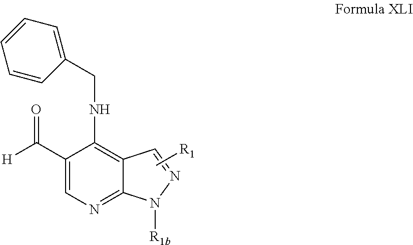
(i) reacting the compound of Formula XLI with hydroxylamine hydrochloride to give a compound of Formula XLII, 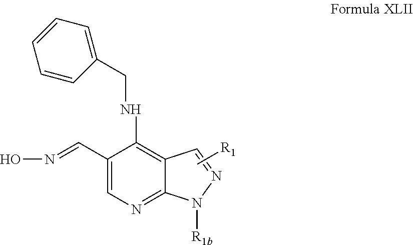
(j) reacting the compound of Formula XLII with a compound of Formula XIII to give a compound of Formula XLIII, 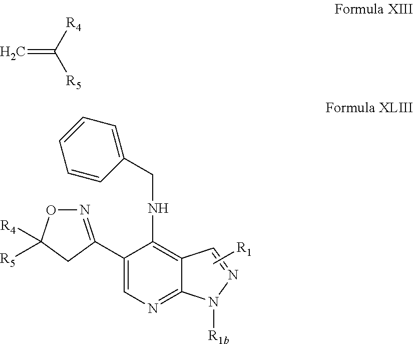
(k) deprotecting the compound of Formula XLIII to give a compound of Formula XLIV, 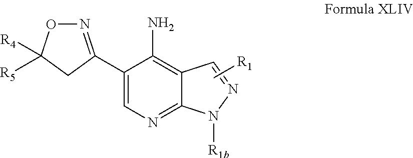
(l) reacting the compound of Formula XLIV with a compound of Formula XLV
R_{1C-X} Formula XLV
to a give compound of Formula XLVI,
wherein R₁ₐ is alkyl, Pr is a protecting group, X is a halogen, R_{1b} is alkyl or cycloalkyl, R_{1c} is aryl or heteroaryl and R₁, R₄ and R₅ are the same as defined in claim 1.

13. A method for the preparation of a compound of Formula XLIIIa, 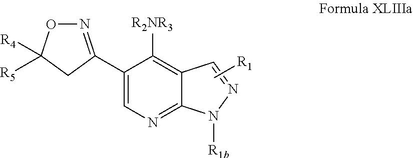
the method comprising,
(a) reacting a compound of Formula XXXIII with a compound of Formula VI to give a compound of Formula XXXVa, 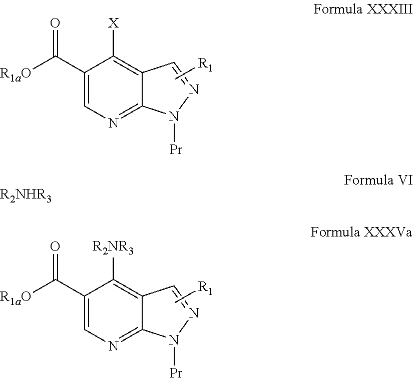
(b) hydrolyzing the compound of Formula XXXVa to give a compound of Formula XXXVIa, 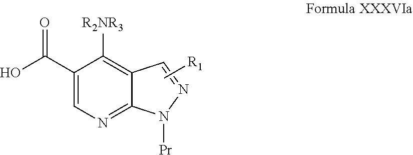
(c) reacting the compound of Formula XXXVIa with a compound of Formula IX to give a compound of Formula XXXVIIa, 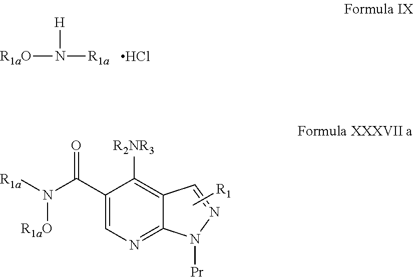
(d) deprotecting the compound of Formula XXXVII a to give a compound of Formula XXXVIIIa, 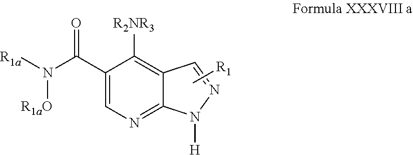
(e) reacting the compound of Formula XXXVIIIa with a compound of Formula XXXIX to give a compound of Formula XLa, 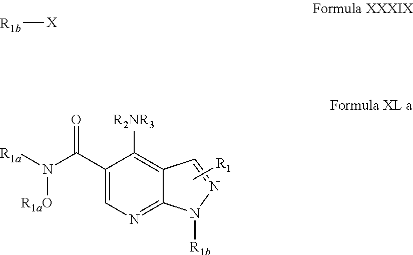
(f) reducing the compound of Formula XLa to give a compound of Formula XLIa, 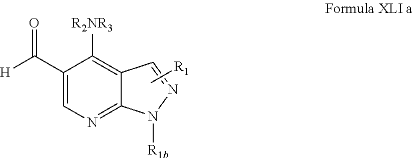
(g) reacting the compound of Formula XLIa with hydroxylamine hydrochloride to give a compound of Formula XLIIa, 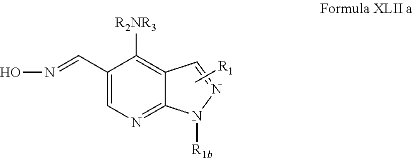
(h) reacting the compound of Formula XLIIa with a compound of Formula XIII 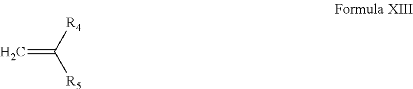
to give a compound of Formula XLIIIa,
wherein X is halogen, R₁ₐ is alkyl, Pr is a protecting group, R_{1b} is alkyl or cycloalkyl and R₁, R₂, R₃, R₄ and R₅ are the same as defined in claim 1.

14. A method for the preparation of a compound of Formula XLVII, 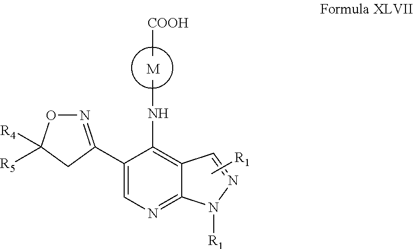
the method comprising, hydrolyzing a compound of Formula XLVIIa 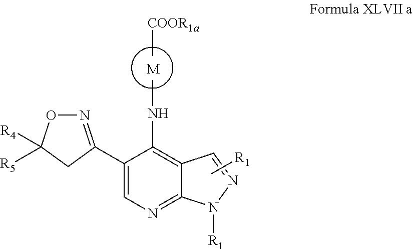
to give a compound of Formula XLVII,
wherein R₁ₐ is alkyl, ring M is cyclobutyl or cyclohexyl ring and R₁, R₄ and R₅ are the same as defined in claim 1.

15. A method for the preparation of compounds of Formulae XLIX, L, LI and LII, 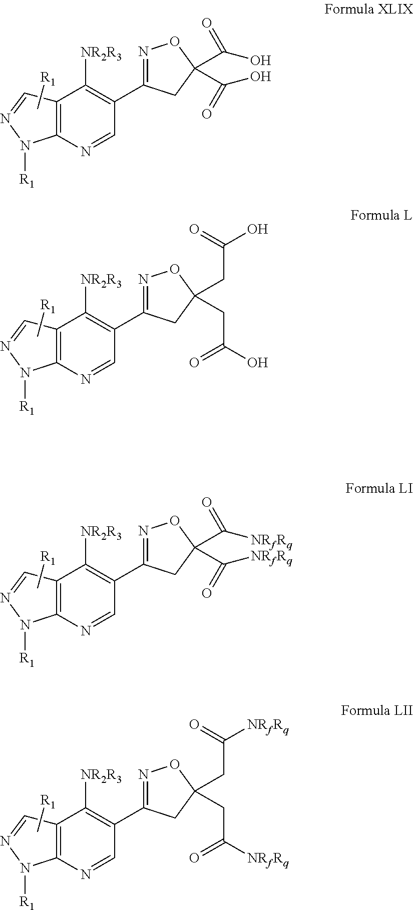
the method comprising,
(a) oxidising a compound of Formula XLVIII to give a compound of Formula XLIX, 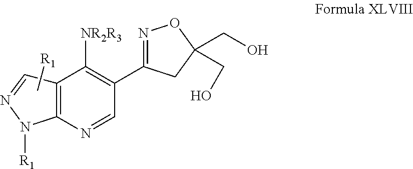
(b) (i) reacting the compound of Formula XLIX with a compound of Formula XVII
R_{f}NHR_{q} Formula XVII
to give a compound of Formula LI,
(ii) halogenating the compound of Formula XLIX to give a compound of Formula XLIXa, 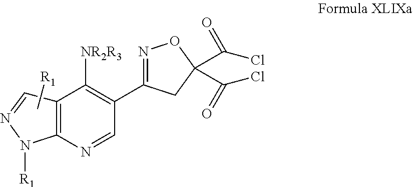
homologating the compound of Formula XLIXa to give a compound of Formula L,
reacting the compound of Formula L with a compound of Formula XVII to give a compound of Formula LII,
wherein R₁, R₂, R₃, R_{f} and R_{q} are the same as defined in claim 1.

16. A method for the preparation of a compound of Formula LIV, 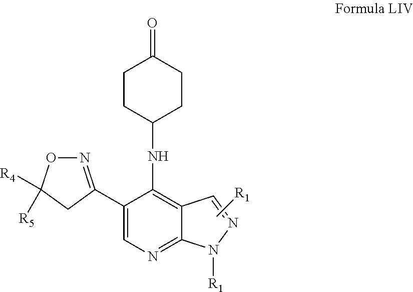
the method comprising, oxidising a compound of Formula LIII 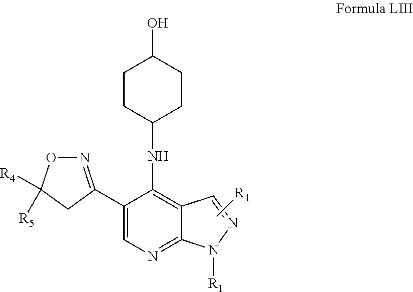
to give a compound of Formula LIV, wherein R₁, R₄ and R₅ are the same as defined in claim 1.

17. A method for treating, preventing, inhibiting or suppressing inflammatory diseases, CNS diseases or autoimmune diseases, in a mammal, comprising administering a therapeutically effective amount of a PDE type 7 inhibitor or dual PDE type 4/PDE type 7 inhibitor having the structure of Formula Ia, 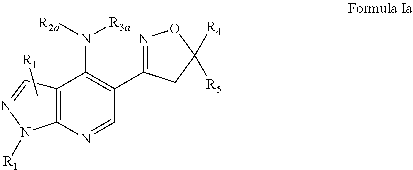
or their pharmaceutically acceptable salts, wherein
R₁ is hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ₐ is hydrogen, alkyl, alkenyl, alkynyl, acyl, cycloalkyl, aryl, aralkenyl, aralkyl, (cycloalkyl) alkyl, heterocyclyl, heteroaryl, (heterocyclyl)alkyl or (heteroaryl) alkyl;
R₃ₐ is cyclopropyl, cyclopentyl, alkyl, alkenyl, alkynyl, acyl, aralkenyl, aralkyl, (cycloalkyl) alkyl, (heterocyclyl)alkyl or (heteroaryl) alkyl;
R₄ and R₅ independently are alkyl, —CN, —(CH₂)ₙC(═O)NR_{f}R_{q} {wherein n is an integer from 0-2 and R_{f} and R_{q} independently are hydrogen, alkyl, alkenyl, cycloalkyl, aryl, aralkyl, heterocyclyl, heteroaryl, (heterocyclyl)alkyl or (heteroaryl)alkyl}, —(CH₂)ₙC(═O)OR_{f} {wherein n and R_{f} are the same as defined above}, —(CH₂)ₙ₁OR_{f} {wherein n1 is an integer from 0-3 and R_{f} is the same as defined above}.

18. A method for the treatment, prevention, inhibition or suppression of multiple sclerosis, AIDS, rejection of transplant, rheumatoid arthritis, bronchitis, chronic obstructive pulmonary disease (COPD), asthma, psoriasis, allergic rhinitis, shock, atopic dermatitis, Crohn's disease, adult respiratory distress syndrome (ARDS), eosinophilic granuloma, allergic conjunctivitis, osteoarthritis, colitis, pancreatitis, and cancer in a mammal comprising administering a therapeutically effective amount of a PDE type 7 inhibitor or dual PDE type 4/PDE type 7 inhibitor having the structure of Formula Ia, 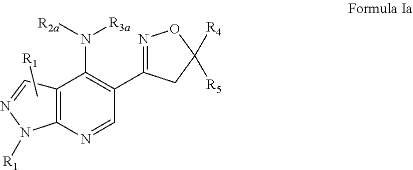
or their pharmaceutically acceptable salts, wherein
R₁ is hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ₐ is hydrogen, alkyl, alkenyl, alkynyl, acyl, cycloalkyl, aryl, aralkenyl, aralkyl, (cycloalkyl) alkyl, heterocyclyl, heteroaryl, (heterocyclyl)alkyl or (heteroaryl) alkyl;
R₃ₐ is cyclopropyl, cyclopentyl, alkyl, alkenyl, alkynyl, acyl, aralkenyl, aralkyl, (cycloalkyl) alkyl, (heterocyclyl)alkyl or (heteroaryl) alkyl;
R₄ and R₅ independently are alkyl, —CN, —(CH₂)ₙC(═O)NR_{f}R_{q} {wherein n is an integer from 0-2 and R_{f} and R_{q} independently are hydrogen, alkyl, alkenyl, cycloalkyl, aryl, aralkyl, heterocyclyl, heteroaryl, (heterocyclyl)alkyl or (heteroaryl)alkyl}, —(CH₂)ₙC(═O)OR_{f} {wherein n and R_{f} are the same as defined above}, —(CH₂)ₙ₁OR_{f} {wherein n1 is an integer from 0-3 and R_{f} is the same as defined above}.

19. The method according to claim 17 or 18, wherein a PDE type 7 inhibitor or dual PDE type 4/PDE type 7 inhibitor is selected from:
Methyl 3-[4-(cyclopropylamino)-1-ethyl-1H-pyrazolo[3,4-b]pyridin-5-yl]-5-methyl-4,5-dihydroisoxazole-5-carboxylate (Compound No. 10);
3-[4-(Cyclopropylamino)-1-ethyl-1H-pyrazolo[3,4-b]pyridin-5-yl]-5-methyl-4,5-dihydroisoxazole-5-carbonitrile (Compound No. 11);
Methyl 3-[4-(cyclopropylamino)-1-ethyl-1H-pyrazolo[3,4-b]pyridin-5-yl]-5-(2-methoxy-2-oxoethyl)-4,5-dihydroisoxazole-5-carboxylate (Compound No. 12);
Methyl 3-[4-(cyclopentylamino)-1,3-dimethyl-1H-pyrazolo[3,4-b]pyridin-5-yl]-5-methyl-4,5-dihydroisoxazole-5-carboxylate (Compound No. 13);
Methyl 3-[4-(cyclopentylamino)-1,3-dimethyl-1H-pyrazolo[3,4-b]pyridin-5-yl]-5-(2-methoxy-2-oxoethyl)-4,5-dihydroisoxazole-5-carboxylate (Compound No. 14);
Methyl 3-[4-(cyclopropylamino)-1,3-dimethyl-1H-pyrazolo[3,4-b]pyridin-5-yl]-5-(2-methoxy-2-oxoethyl)-4,5-dihydroisoxazole-5-carboxylate (Compound No. 15);
Methyl 3-[4-(cyclopentylamino)-1-ethyl-1H-pyrazolo[3,4-b]pyridin-5-yl]-5-methyl-4,5-dihydroisoxazole-5-carboxylate (Compound No. 16);
Methyl 3-[4-(cyclopropylamino)-1,3-dimethyl-1H-pyrazolo[3,4-b]pyridin-5-yl]-5-methyl-4,5-dihydroisoxazole-5-carboxylate (Compound No. 17);
Methyl 3-[4-(cyclopentylamino)-1-ethyl-1H-pyrazolo[3,4-b]pyridin-5-yl]-5-(2-methoxy-2-oxoethyl)-4,5-dihydroisoxazole-5-carboxylate (Compound No. 18);
5-(Carboxymethyl)-3-[4-(cyclopropylamino)-1-ethyl-1H-pyrazolo[3,4-b]pyridin-5-yl]-4,5-dihydroisoxazole-5-carboxylic acid (Compound No. 19);
3-[4-(Cyclopropylamino)-1-ethyl-1H-pyrazolo[3,4-b]pyridin-5-yl]-5-methyl-4,5-dihydroisoxazole-5-carboxylic acid (Compound No. 20);
{3-[4-(Cyclopropylamino)-1-ethyl-1H-pyrazolo[3,4-b]pyridin-5-yl]-5-methyl-4,5-dihydroisoxazol-5-yl}methanol (Compound No. 21);
2-[3-[4-(Cyclopropylamino)-1-ethyl-1H-pyrazolo[3,4-b]pyridin-5-yl]-5-(hydroxymethyl)-4,5-dihydroisoxazol-5-yl]ethanol (Compound No. 22);
{3-[4-(Cyclopentylamino)-1,3-dimethyl-1H-pyrazolo[3,4-b]pyridin-5-yl]-5-methyl-4,5-dihydroisoxazol-5-yl}methanol (Compound No. 23);
2-[3-[4-(Cyclopentylamino)-1,3-dimethyl-1H-pyrazolo[3,4-b]pyridin-5-yl]-5-(hydroxymethyl)-4,5-dihydroisoxazol-5-yl]ethanol (Compound No. 24);
{3-[4-(Cyclopropylamino)-1,3-dimethyl-1H-pyrazolo[3,4-b]pyridin-5-yl]-5-methyl-4,5-dihydroisoxazol-5-yl}methanol (Compound No. 25);
{3-[4-(Cyclopentylamino)-1-ethyl-1H-pyrazolo[3,4-b]pyridin-5-yl]-5-methyl-4,5-dihydroisoxazol-5-yl}methanol (Compound No. 26);
5-(2-Amino-2-oxoethyl)-3-[4-(cyclopropylamino)-1-ethyl-1H-pyrazolo[3,4-b]pyridin-5-yl]-4,5-dihydroisoxazole-5-carboxamide (Compound No. 27);
3-[4-(Cyclopropylamino)-1-ethyl-1H-pyrazolo[3,4-b]pyridin-5-yl]-5-methyl-4,5-dihydroisoxazole-5-carboxamide (Compound No. 28);
3-[4-(Cyclopentylamino)-1,3-dimethyl-1H-pyrazolo[3,4-b]pyridin-5-yl]-N,5-dimethyl-4,5-dihydroisoxazole-5-carboxamide (Compound No. 29);
3-[4-(Cyclopentylamino)-1,3-dimethyl-1H-pyrazolo[3,4-b]pyridin-5-yl]-5-methyl-4,5-dihydroisoxazole-5-carboxamide (Compound No. 30);
5-(2-Amino-2-oxoethyl)-3-[4-(cyclopentylamino)-1,3-dimethyl-1H-pyrazolo[3,4-b]pyridin-5-yl]-4,5-dihydroisoxazole-5-carboxamide (Compound No. 31);
N-cyclopropyl-3-[4-(cyclopropylamino)-1,3-dimethyl-1H-pyrazolo[3,4-b]pyridin-5-yl]-5-[2-(cyclopropylamino)-2-oxoethyl]-4,5-dihydroisoxazole-5-carboxamide (Compound No. 32); and
5-(2-Amino-2-oxoethyl)-3-[4-(cyclopropylamino)-1,3-dimethyl-1H-pyrazolo[3,4-b]pyridin-5-yl]-4,5-dihydroisoxazole-5-carboxamide (Compound No. 33).
or its pharmaceutically acceptable salts, pharmaceutically acceptable solvates, stereoisomers, tautomers, racemates, regioisomers, geometric isomers, prodrugs, metabolites, polymorphs or N-oxides.
